(19) United States
(12) Patent Application Publication
(10) Pub. No.: US 2026/0297065 A1
(43) Pub. Date: Oct. 1, 2026

(54) AROMATIC SUBSTITUENT-CONTAINING (HETERO) ARYL-IMIDAZOLE COMPOUND, AND PREPARATION METHOD THEREFOR AND USE THEREOF

(71) Applicant: OPEN SOURCE THERAPEUTICS, Suzhou (CN)

(72) Inventors: Lisheng DENG, Suzhou (CN); Fangrui WU, Suzhou (CN); Zhixiong MA, Suzhou (CN)

(21) Appl. No.: 19/140,283

(22) PCT Filed: Jul. 18, 2023
(86) PCT No.: PCT/CN2023/107945

§ 371 Date: Jun. 12, 2026

(30) Foreign Application Priority Data
Jul. 20, 2022 (CN) 202210857338.3

(51) Int. Cl. C07D 403/14; A61K 31/4184; A61K 31/4439; A61K 31/4545; A61K 31/501; A61K 31/506; A61K 31/5377; C07D 235/18; C07D 401/04; C07D 401/14; C07D 403/04; C07D 405/14; C07D 413/14; C07D 471/04

(52) U.S. Cl. CPC C07D 403/14; A61K 31/4184; A61K 31/4439; A61K 31/4545; A61K 31/501; A61K 31/506; A61K 31/5377; C07D 235/18; C07D 401/04; C07D 401/14; C07D 403/04; C07D 405/14; C07D 413/14; C07D 471/04

(57) ABSTRACT

The present application relates to an aromatic substituent-containing heteroaryl- or phenyl-imidazole compound, and a stereoisomer, hydrate, solvate, pharmaceutically-acceptable salt or prodrug thereof. The present application also relates to a method for preparing the aromatic substituent-containing heteroaryl- or phenyl-imidazole compound, and a use of the aromatic substituent-containing heteroaryl- or phenyl-imidazole compound as a soluble epoxide hydrolase inhibitor. The aromatic substituent-containing heteroaryl- or phenyl-imidazole compound herein exhibits extremely high inhibitory activity on soluble epoxide hydrolases, and the IC₅₀ value is even lower at the nanomolar level.

### TECHNICAL FIELD

The present application relates to the technical field of biomedicine and soluble epoxide hydrolase inhibitors. Specifically, the present application relates to an aromatic substituent-containing (hetero)aryl imidazole compound, a method for preparing the aromatic substituent-containing (hetero)aryl imidazole compound, and use of the aromatic substituent-containing (hetero)aryl imidazole compound as a soluble epoxide hydrolase inhibitor.

### BACKGROUND ART

Soluble epoxide hydrolase (sEH) plays a central role in the metabolism of bioactive lipid signaling molecules. sEH can specifically convert the substrate epoxyeicosatrienoic acid (EET) into dihydroxyeicosatrienoic acid (DET) with lower biological activity. These EETs are ubiquitously present in endothelial cells, kidney and lung. Compared to many proinflammatory end products of the prostaglandin and leukotriene pathways, EETs possess anti-inflammatory, analgesic, antihypertensive, cardioprotective, and organ protective properties. Although EETs have a very positive effect on human health, due to the high catalytic activity of sEH in the body, EETs cannot be effectively enriched in the body to more significantly exert their biological effects. Therefore, developing suitable sEH inhibitors to inhibit the hydrolysis of EETs and promote their enrichment in the body to enhance their anti-inflammatory and other effects has become a key exploration direction for scientific researchers. Studies have shown that inhibition of sEH can significantly reduce blood pressure in mammals and promote the resolution of inflammation by reducing the production of inflammatory factors (NO), cytokines and lipid mediators. In the early stage, several sEH inhibitors entered the clinical trial stage, but currently no sEH inhibitor drugs have been approved for marketing. One of the important reasons is that the catalytic activity of sEH is very high and its concentration in the human body is also very high. Therefore, compounds with excellent activity and pharmacokinetic properties are needed to effectively inhibit sEH in vivo and achieve the expected drug effect.

Recently, WO2021242790A1 disclosed a benzimidazole compound that can be used as a soluble epoxide hydrolase inhibitor. In an in vitro activity test using human sEH and its substrate PHOME as model compounds, the IC₅₀ values of some of the optimal compounds can reach 1 μM.

In short, developing drug molecules with better activity and pharmacokinetic properties has become an urgent problem that needs to be solved in the development of sEH inhibitors.

### SUMMARY OF THE INVENTION

The object of the present application is to provide an aromatic substituent-containing (hetero)aryl imidazole compound, which can be used as a soluble epoxide hydrolase inhibitor. Specifically, in the aromatic substituent-containing (hetero)aryl imidazole compound, at least one unsaturated carbon atom in the (hetero)aromatic ring of the (hetero)aryl imidazole structure is directly connected to an unsaturated carbon atom of the first aromatic substituent, and a carbon atom adjacent to two N atoms in the imidazole ring of the (hetero)aryl imidazole structure is directly connected to an unsaturated carbon atom of the second aromatic substituent.

The object of the present application is also to provide a method for preparing the aromatic substituent-containing (hetero)aryl imidazole compound as described above.

The object of the present application is also to provide use of the aromatic substituent-containing (hetero)aryl imidazole compound as described above as a soluble epoxide hydrolase inhibitor.

The object of the present application is also to provide a pharmaceutical composition, which comprises the aromatic substituent-containing (hetero)aryl imidazole compound as described above and a pharmaceutically acceptable carrier, auxiliary material or excipient.

The object of the present application is also to provide a kit, which comprises the aromatic substituent-containing (hetero)aryl imidazole compound or pharmaceutical composition as described above. The object of the present application is also to provide use of the aromatic substituent-containing (hetero)aryl imidazole compound as described above in the preparation of a drug for sEH-related disease.

The object of the present application is also to provide a method for preventing or treating sEH-related disease.

In order to solve the above technical problems, this application provides the following technical solutions.

In the first aspect, the present application provides an aromatic substituent-containing (hetero)aryl imidazole compound, and a stereoisomer, hydrate, solvate, pharmaceutically acceptable salt or prodrug thereof, characterized in that the aromatic substituent-containing (hetero)aryl imidazole compound has a structure represented by the following general formula IA or general formula IB:

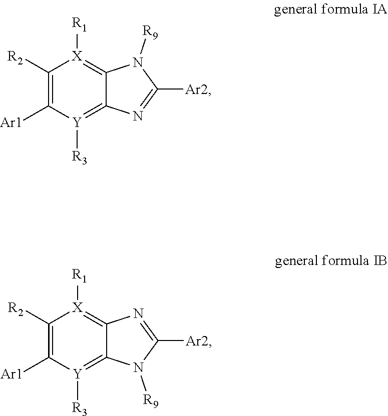

wherein, X and Y each independently represent C or N;
R₁, R₂ and R₃ are each independently selected from a group consisting of hydrogen, deuterium, tritium, halogen, substituted or unsubstituted straight-chain alkyl, substituted or unsubstituted branched alkyl, substituted or unsubstituted heteroalkyl, substituted or unsubstituted cycloalkyl, substituted or unsubstituted heterocycloalkyl, cyano, trifluoromethyl, alkoxy, cycloalkyloxy, aryloxy, substituted or unsubstituted amino, substituted or unsubstituted thiol, substituted or unsubstituted sulfonyl, substituted or unsubstituted sulfuryl, substituted or unsubstituted alkenyl, substituted or unsubstituted alkynyl, substituted or unsubstituted heteroalkenyl, substituted or unsubstituted heteroalkynyl, substituted or unsubstituted aryl, and substituted or unsubstituted heteroaryl, or isotopic substituents thereof;
R₉ is selected from a group consisting of hydrogen, deuterium, tritium, halogen, substituted or unsubstituted straight chain alkyl, substituted or unsubstituted branched chain alkyl, substituted or unsubstituted heteroalkyl, substituted or unsubstituted cycloalkyl, substituted or unsubstituted heterocycloalkyl, cyano, trifluoromethyl, alkoxy, cycloalkyloxy, aryloxy, substituted or unsubstituted amino, substituted or unsubstituted thiol, substituted or unsubstituted sulfonyl, substituted or unsubstituted sulfuryl, substituted or unsubstituted alkenyl, substituted or unsubstituted alkynyl, substituted or unsubstituted heteroalkenyl, substituted or unsubstituted heteroalkynyl, substituted or unsubstituted aryl, and substituted or unsubstituted heteroaryl, or isotopic substituents thereof, or a group of a prodrug of the parent structure;
Ar1 and Ar2 are each independently selected from a group consisting of substituted or unsubstituted aryl, substituted or unsubstituted heteroaryl;
wherein, at least one unsaturated carbon atom in the (hetero)aromatic ring of the (hetero)aryl imidazole structure is directly connected to an unsaturated carbon atom of Ar1, and a carbon atom adjacent to two N atoms in the imidazole ring of the (hetero)aryl imidazole structure is directly connected to an unsaturated carbon atom of Ar2.

In another preferred embodiment, X and Y are C or N, wherein when X and Y are N, R₁ and R₃ do not exist;

R₁, R₂ and R₃ are each independently selected from a group consisting of hydrogen, deuterium, tritium, halogen, alkyl, haloalkyl, cycloalkyl, alkoxy, heterocycloalkyl, cyano, aryl, heteroaryl;
R₉ is hydrogen, deuterium, tritium, alkyl, haloalkyl, alkoxy, haloalkoxy, or sulfuryl;
Ar1 is selected from a group consisting of substituted or unsubstituted aryl, substituted or unsubstituted heteroaryl, wherein the substituted refers to substitution by one or more substituents selected from a group consisting of cyano, carboxyl, ester group, hydroxyl, alkyl, haloalkyl, alkoxy, cycloalkyloxy, heterocyclyloxy, halogen, heterocyclyl, alkylene-heterocyclyl, cycloalkyl, —COalkyl, —S(O)₂-alkyl, ═O, aryl, heteroaryl, haloalkoxy, —NRaRb, -alkyleneNRaRb, —SO₂NRaRb, —CONRaRb, and each of the above substituents may be further substituted by one or more halogen, hydroxyl, amino, alkyl, haloalkyl, cycloalkyl, halocycloalkyl, heterocyclyl, halogenated heterocyclyl, alkoxy, haloalkoxy, cycloalkyloxy, halocycloalkyloxy, heterocyclyloxy, halogenated heterocyclyloxy;
Ar2 is selected from a group consisting of substituted or unsubstituted aryl, substituted or unsubstituted heteroaryl, wherein the substituted refers to substitution by one or more substituents selected from a group consisting of halogen, heterocyclyl, haloalkyl, ═O, —SO₂alkyl, —SO₂NRaRb, —CONRaRb, alkoxy, alkyl, haloalkoxy, alkyl-substituted thiol, haloalkyl-substituted thiol, cyano, ester group, aryl, heteroaryl, NRaRb, and each of the above substituents may be further substituted by one or more halogen, hydroxy, alkyl, haloalkyl, cycloalkyl, halocycloalkyl, heterocyclyl, haloheterocyclyl, alkoxy, haloalkoxy, cycloalkyloxy, halocycloalkyloxy, heterocyclyloxy, haloheterocyclyloxy;
each Ra and each Rb, at each occurrence, are independently selected from: H, alkyl, heteroalkyl, heterocyclyl, acyl, sulfuryl, sulfonyl; or Ra and Rb, at each occurrence, optionally form a 3-8 membered heterocycle with the connected N, and the above alkyl and 4-8 membered heterocycle may optionally be substituted by one or more substituents selected from the following group: OH, COOH, CONH₂, COalkyl, alkoxy, alkyl, haloalkoxy, haloalkyl, aryl, heteroaryl, amino, monoalkylamino, dialkylamino.

In another preferred embodiment, X and Y are C; R₁, R₂ and R₃ are each independently selected from a group consisting of hydrogen, deuterium, tritium, halogen, alkyl, and halogenated alkyl; R₉ is hydrogen;
Ar1 is a substituted or unsubstituted heteroaryl, wherein the substituted refers to substitution by one or more substituents selected from a group consisting of NRaRb, -alkylene NRaRb, cyano, carboxyl, ester group, hydroxyl, alkyl, haloalkyl, alkoxy, haloalkoxy, halogen, heterocyclyl, alkylene-heterocyclyl, cycloalkyl, —COalkyl, —SO₂alkyl, ═O, and each of the above substituents may be further substituted by one or more halogen, hydroxyl, amino, alkyl, haloalkyl, alkoxy, haloalkoxy, cycloalkyloxy, halocycloalkyloxy, heterocyclyloxy, and haloheterocyclyloxy;
Ar2 is substituted or unsubstituted aryl, substituted or unsubstituted heteroaryl, wherein the substituted refers to substitution by one or more substituents selected from a group consisting of halogen, heterocyclyl, ═O, haloalkyl, —SO₂alkyl, —SO₂NRaRb, —CONRaRb, alkoxy, NRaRb, alkyl, cyano, ester group, thiol substituted by haloalkyl, and each of the above substituents may be further substituted by one or more halogen, hydroxyl, alkyl, haloalkyl, alkoxy, haloalkoxy, cycloalkyloxy, halocycloalkyloxy, heterocyclyloxy, halogenated heterocyclyloxy;
each Ra and each Rb, at each occurrence, are independently selected from: H, alkyl, heterocyclyl; or at each occurrence, Ra and Rb are optionally combined with the connected N to form a 3-8 membered heterocycle, and the alkyl and heterocycle may be optionally substituted with one or more substituents selected from the following group: OH, COOH, CONH₂, COalkyl, alkoxy, alkyl, haloalkoxy, haloalkyl, aryl, heteroaryl, amino, monoalkylamino, dialkylamino.

In another preferred embodiment, X and Y are C; R₁, R₂ and R₃ are each independently selected from a group consisting of hydrogen, deuterium, tritium, F, Cl, Br, I; R₉ is hydrogen;
Ar1 is a substituted or unsubstituted 5-8 membered heteroaryl, wherein the substituted refers to substitution by one or more substituents selected from a group consisting of NRaRb, —C₁₋₄ alkylene NRaRb, cyano, carboxyl, C₁₋₆ alkyl, halogenated C₁₋₆ alkyl, C₁₋₆ alkoxy, halogen, 3-8 membered heterocyclyl, —C₁₋₄ alkylene-3-8 membered heterocyclyl, C₃₋₆ cycloalkyl, —CO—C₁₋₆ alkyl, —COOC₁₋₆ alkyl, —SO₂—C₁₋₆ alkyl, ═O, hydroxyl;
Ar2 is substituted or unsubstituted phenyl, substituted or unsubstituted 5-9 membered heteroaryl, wherein the substituted refers to substitution by one or more substituents selected from a group consisting of halogen, 3-8 membered heterocyclyl, ═O, halogenated C₁₋₆ alkyl, —SO₂C₁₋₆ alkyl, C₁₋₆ alkyl, —SO₂NRaRb, —CONRaRb, —NRaRb, C₁₋₆ alkoxy, C₁₋₆ haloalkoxy;
each Ra and each Rb, at each occurrence, are independently selected from: H, C₁₋₆ alkyl, 3-6 membered heterocyclyl; or Ra and Rb, at each occurrence, optionally form a 3-6 membered heterocycle with the connected N, and the above alkyl and heterocyclyl may be optionally substituted by one or more substituents selected from the following group: OH, COOH, CONH₂, COC₁₋₆ alkyl, C₁₋₆ alkoxy.

In another preferred embodiment, X and Y are C;
R₁, R₂ and R₃ are each independently selected from a group consisting of hydrogen, deuterium, tritium, F, Cl, Br;
R₉ is hydrogen;
Ar1 is substituted or unsubstituted 5-6 membered heteroaryl, wherein the substituted refers to substitution by one or more substituents selected from a group consisting of amino, methylamino, dimethylamino, methyl, ethyl, n-propyl, isopropyl, trifluoromethyl, difluoromethyl, monofluoromethyl, 1-methyl-1-fluoroethyl, methoxy, ethoxy, F, Cl, Br, pyrrolyl, morpholinyl, piperidinyl, piperazinyl, 4-methylpiperazinyl, cyclopropyl, cyclobutyl, cyclohexyl, acetyl, propionyl, butyryl, methylsulfonyl, ethylsulfonyl, propylsulfonyl, cyclopropylsulfonyl, ═O, carboxyl, cyano, hydroxyl,

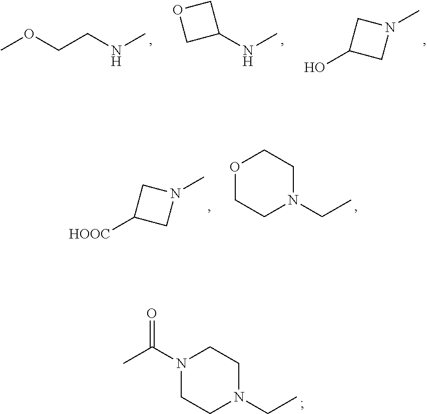

Ar2 is substituted or unsubstituted phenyl, substituted or unsubstituted 5-9 membered heteroaryl, wherein the substituted refers to substitution by one or more substituents selected from a group consisting of trifluoromethyl, difluoromethyl, monofluoromethyl, methylsulfonyl, trifluoromethylthiol, cyano, dimethylamino, methylsulfonyl, ethylsulfonyl, propylsulfonyl, —SO₂NRaRb, —CONRaRb, methoxy, trifluoromethoxy, ethoxy, trifluoroethoxy, fluorine, chlorine, bromine, methyl, ethyl, n-propyl, isopropyl, methoxycarbonyl, ethoxycarbonyl, ═O, morpholinyl,

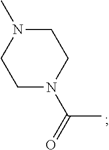

Ra, Rb: H, methyl, ethyl, n-propyl, isopropyl, oxetanyl; or Ra, Rb and the connected N form morpholinyl, 4-methylpiperazinyl, pyrrolidyl or piperidinyl, and the above groups may be optionally substituted by one or more substituents selected from the following group: methoxy, hydroxyl, carboxyl, CONH₂.

In another preferred embodiment, Ar1 is a substituted or unsubstituted 5-7 heteroaryl containing 1 or 2 nitrogen atoms; Ar2 is a substituted or unsubstituted phenyl, and the definitions of each substitution are the same as above.

In another preferred embodiment, Ar1 is substituted or unsubstituted pyridyl, pyridazinyl, pyrimidinyl, pyrazinyl, pyrazolyl, imidazolyl or pyrrolyl, and the substitution is as defined above.

In another preferred embodiment, Ar2 is substituted or unsubstituted phenyl, pyridyl, pyrimidinyl, benzoxazolyl,

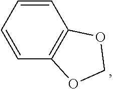

and the substitution is as defined above.

In one embodiment of the first aspect, the aromatic substituent-containing (hetero)aryl imidazole compound has a structure represented by the following general formula IIA or IIB:

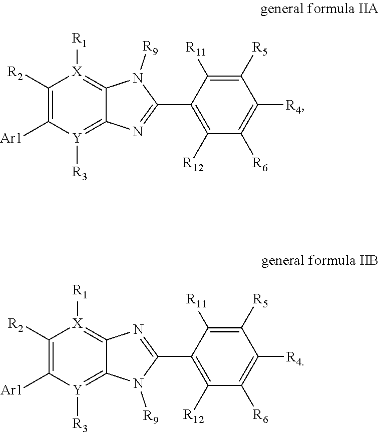

wherein, R₄, R₅, R₆, R₁₁ and R₁₂ are each independently selected from the following group: hydrogen, deuterium, tritium, halogen, substituted or unsubstituted straight-chain alkyl, substituted or unsubstituted branched-chain alkyl, substituted or unsubstituted heteroalkyl, substituted or unsubstituted cycloalkyl, substituted or unsubstituted heterocycloalkyl, cyano, trifluoromethyl, alkoxy, cycloalkyloxy, aryloxy, substituted or unsubstituted amino, substituted or unsubstituted thiol, substituted or unsubstituted sulfonyl, substituted or unsubstituted sulfuryl, substituted or unsubstituted alkenyl, substituted or unsubstituted alkynyl, substituted or unsubstituted heteroalkenyl, substituted or unsubstituted heteroalkynyl, substituted or unsubstituted aryl, and substituted or unsubstituted heteroaryl, or isotopic substituents thereof.

In one embodiment of the first aspect, R₁ and R₂ are connected to form a five-membered ring, a six-membered ring, a seven-membered ring or an eight-membered ring;
and/or, R₄ and R₅ are connected to form a five-membered ring, a six-membered ring, a seven-membered ring or an eight-membered ring;
and/or, R₄ and R₆ are connected to form a five-membered ring, a six-membered ring, a seven-membered ring or an eight-membered ring.

In another preferred embodiment, R₄, R₅, R₆, R₁₁ and R₁₂ are each independently selected from the following group: halogen, heterocyclyl, ═O, haloalkyl, haloalkoxy, —SO₂alkyl, —SO₂NRaRb, —CONRaRb, alkoxy, NRaRb, alkyl, cyano, haloalkyl substituted mercapto, ester group; adjacent substituents and the connected C form a substituted or unsubstituted heterocycle, and the substituted heterocycle has one or more substituents selected from halogen, alkyl, ═O;
each Ra and each Rb, at each occurrence, are independently selected from: H, alkyl, heterocyclic; or at each occurrence, Ra and Rb optionally form a heterocycle with the connected N, and the alkyl and heterocycle may optionally be substituted with one or more substituents selected from the following group: OH, COOH, CONH₂, COalkyl, alkoxy, alkyl, haloalkoxy, haloalkyl, aryl, heteroaryl, amino, monoalkylamino, dialkylamino.

In another preferred embodiment, R₄, R₅, R₆, R₁₁ and R₁₂ are each independently selected from the following group: halogen, 3-8 membered heterocyclyl, ═O, halogenated C₁₋₆ alkyl, halogenated C₁₋₆ alkoxy, —SO₂C₁₋₆ alkyl, —SO₂NRaRb, —CONRaRb, C₁₋₆ alkoxy, NRaRb, C₁₋₆ alkyl, cyano, —S-halogenated C₁₋₆ alkyl, —COOC₁₋₆ alkyl; adjacent substituents form a substituted or unsubstituted 3-8 membered heterocycle with the connected C, and the substituted heterocycle has one or more substituents selected from halogen, C₁₋₆ alkyl, ═O;
each Ra and each Rb, at each occurrence, are independently selected from: H, C₁₋₆ alkyl, 3-8 membered heterocyclyl; or Ra and Rb, at each occurrence, optionally form a 3-8 membered heterocycle with the connected N, and the alkyl and the heterocycle may optionally be substituted by one or more substituents selected from the following group: OH, COOH, CONH₂, COC₁₋₆ alkyl, C₁₋₆ alkoxy, C₁₋₆ alkyl, halogenated C₁₋₆ alkoxy, halogenated C₁₋₆ alkyl, phenyl, 3-8 membered heteroaryl, amino, mono C₁₋₆ alkylamino, di C₁₋₆ alkylamino.

In another preferred embodiment, R₄ is selected from the following group: H, halogen, —SO₂NRaRb, C₁₋₆ alkoxy, —CONRaRb, ═SO₂C₁₋₆ alkyl, halogenated C₁₋₆ alkyl, halogenated C₁₋₆ alkoxy; each Ra and each Rb are independently selected from the following groups at the occurrence: H, C₁₋₆ alkyl, 3-8 membered heterocyclyl; or at each occurrence, Ra and Rb optionally form 3-8 membered heterocycle with the adjacent N atom, and the alkyl and the heterocycle may be optionally substituted by one or more substituents selected from the following groups: OH, COOH, CONH₂, COC₁₋₆ alkyl, C₁₋₆ alkoxy, C₁₋₆ alkyl, halogenated C₁₋₆ alkoxy, halogenated C₁₋₆ alkyl, phenyl, 3-8 membered heteroaryl, amino, mono-C₁₋₆ alkylamino, di-C₁₋₆ alkylamino.

In another preferred embodiment, R₅ and R₆ are selected from the following groups: H, halogen, halogenated C₁₋₆ alkyl, halogenated C₁₋₆ alkoxy, and a 3-8 membered heterocyclyl, and the heterocyclyl may be optionally substituted by one or more substituents selected from the following group: COC₁₋₆ alkyl, OH, COOH, C₁₋₆ alkoxy, C₁₋₆ alkyl, and halogen.

In another preferred embodiment, R₅ and R₆ are selected from the following group: H, fluorine, chlorine, bromine, trifluoromethyl, difluoromethyl, cyano, morpholinyl,

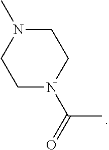

In another preferred embodiment, R₁₁ and R₁₂ are each independently selected from the following group: H.

In one embodiment of the first aspect, Ar1 has a structure represented by the following general formula III:

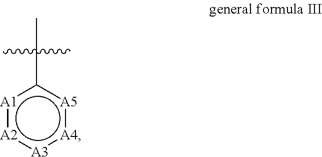

wherein A1, A2, A3, A4 and A5 are each independently selected from a group consisting of CR, N, N—R, S and O, wherein R is each independently selected from a group consisting of hydrogen, deuterium, tritium, halogen, cyano, trifluoromethyl, alkoxy, cycloalkyloxy, aryloxy, substituted or unsubstituted amino, substituted or unsubstituted thiol, substituted or unsubstituted sulfonyl, substituted or unsubstituted sulfuryl, substituted or unsubstituted straight-chain alkyl, substituted or unsubstituted branched-chain alkyl, substituted or unsubstituted heteroalkyl, substituted or unsubstituted cycloalkyl, substituted or unsubstituted heterocycloalkyl, substituted or unsubstituted alkenyl, substituted or unsubstituted alkynyl, substituted or unsubstituted heteroalkenyl, substituted or unsubstituted heteroalkynyl, substituted or unsubstituted aryl, and substituted or unsubstituted heteroaryl, or isotopic substituents thereof.

In another preferred embodiment, A1, A2, A3, A4, and A5 are each independently selected from: CR, N, N—R, S and O, wherein R at each occurrence is independently selected from: hydrogen, deuterium, tritium, halogen, cyano, carboxyl, alkyl, hydroxyl, NRaRb, -alkylene NRaRb, alkoxy, haloalkyl, haloalkoxy, heterocycloalkyl, alkylene heterocycloalkyl, ═O;
each Ra and each Rb, at each occurrence, are independently selected from: H, alkyl, heterocyclyl; or Ra and Rb, at each occurrence, optionally form a 5-8 membered heterocycle with the connected N, and the above alkyl and 5-8 membered heterocycle may be optionally substituted with one or more substituents selected from the following group: OH, COOH, CONH₂, COalkyl, alkoxy, alkyl, haloalkoxy, haloalkyl, aryl, heteroaryl, amino, monoalkylamino, dialkylamino.

In another preferred embodiment, A1, A2, A3, A4, and A5 are each independently selected from: CR, N, wherein R is selected from: hydrogen, deuterium, tritium, F, Cl, Br, cyano, carboxyl, hydroxyl, C₁₋₆ alkyl, C₁₋₆ haloalkyl, C₁₋₆ alkoxy, 5-7 membered heterocycloalkyl, C₁₋₆ alkyl substituted 5-7 membered heterocycloalkyl, ═O, —NRaRb, -alkyleneNRaRb;
Ra and Rb, at each occurrence, are each independently selected from: H, C₁₋₆ alkyl, 5-7 membered heterocyclyl; or Ra and Rb, at each occurrence, optionally form a 5-7 membered heterocycle with the connected N, and the above alkyl and heterocycle are optionally substituted by one or more substituents selected from the following group: OH, COOH, CONH₂, COC₁₋₆ alkyl, C₁₋₆ alkyl.

In another preferred embodiment, A1 and A5 are each independently selected from: CR, N, S and O, wherein R is selected from: hydrogen, deuterium, tritium, halogen, cyano, carboxyl, alkyl, NH₂, —NHalkyl, —N(alkyl)₂, alkoxy, haloalkyl;
A2 and A4 are each independently selected from: CR, N, wherein R is selected from: hydrogen, deuterium, tritium, halogen, alkyl, amino;
A3 is selected from: CR, N, wherein R is selected from: hydrogen, deuterium, tritium, halogen, alkyl, —NRaRb, -alkyleneNRaRb, heterocycloalkyl, alkyleneheterocycloalkyl, alkoxy, ═O, haloalkyl;
Ra and Rb, at each occurrence, are each independently selected from: H, alkyl, heterocyclyl; or Ra and Rb, at each occurrence, optionally form a heterocycle with the connected N, and the above alkyl and heterocycle are optionally substituted with one or more substituents selected from the following group: OH, COOH, CONH₂, COalkyl, alkyl.

In another preferred embodiment, A1 and A5 are each independently selected from: CR, N, wherein R is selected from: hydrogen, deuterium, tritium, F, Cl, Br, cyano, carboxyl, C₁₋₆ alkyl, C₁₋₆ haloalkyl, —N(C₁₋₆ alkyl)(C₁₋₆ alkyl), —NH(C₁₋₆ alkyl), C₁₋₆ alkoxy, NH₂;
A2 and A4 are each independently selected from: CR, N, wherein R is selected from: hydrogen, deuterium, tritium, NH₂, —N(C₁₋₆ alkyl)(C₁₋₆ alkyl), —NH(C₁₋₆ alkyl);
A3 is selected from: CR, N, wherein R is selected from: hydrogen, deuterium, tritium, NH₂, C₁₋₆ alkyl, C₁₋₆ alkoxy, F, Cl, Br, C₁₋₆ haloalkyl, 5-7 membered heterocycloalkyl, C₁₋₆ alkyl substituted 5-7 membered heterocycloalkyl, ═O, —NRaRb, -alkyleneNRaRb;

Ra and Rb, at each occurrence, are each independently selected from: H, C₁₋₆ alkyl, 5-7 membered heterocyclyl; or Ra and Rb, at each occurrence, optionally form a 5-7 membered heterocycle with the connected N, and the above alkyl and heterocycle are optionally substituted by one or more substituents selected from the following group: OH, COOH, CONH₂, COC₁₋₆ alkyl, C₁₋₆ alkyl.

In another preferred embodiment, A1 and A5 are each independently selected from: CR, N, wherein R is selected from: hydrogen, deuterium, tritium, cyano, F, Cl, Br, carboxyl, methyl, ethyl, n-propyl, isopropyl, trifluoromethyl, difluoromethyl, monofluoromethyl, methoxy, ethoxy, NH₂, —NHCH₃, —NH CH₂CH₃, N(CH₃)(CH₂CH₃), N(CH₂CH₃)₂;
A2 and A4 are each independently selected from: CR, N, wherein R is selected from: hydrogen, deuterium, tritium, NH₂, —NHCH₃, —NH CH₂CH₃, N(CH₃)(CH₂CH₃), N(CH₂CH₃)₂;
A3 is selected from: CR, N, wherein R is selected from: hydrogen, deuterium, tritium, methyl, ethyl, n-propyl, isopropyl, methoxy, ethoxy, F, Cl, Br, trifluoromethyl, difluoromethyl, monofluoromethyl, morpholinyl, piperidinyl, piperazinyl, methylpiperazinyl, ═O, NH₂, —NHCH₃, —NH CH₂CH₃, N(CH₃)(CH₂CH₃), N(CH₂CH₃)₂,

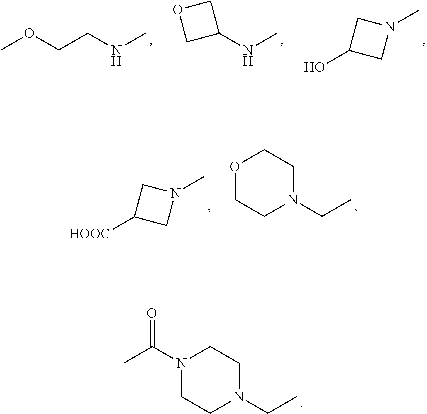

In one embodiment of the first aspect, Ar1 has a structure represented by the following general formula IV:

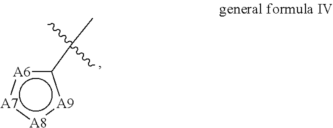

wherein A6, A7, A8, A8 and A9 are each independently selected from a group consisting of CR, N, N—R, S and O, wherein R is each independently selected from a group consisting of hydrogen, deuterium, tritium, halogen, cyano, trifluoromethyl, alkoxy, cycloalkyloxy, aryloxy, substituted or unsubstituted amino, substituted or unsubstituted thiol, substituted or unsubstituted sulfonyl, substituted or unsubstituted sulfuryl, substituted or unsubstituted straight-chain alkyl, substituted or unsubstituted branched-chain alkyl, substituted or unsubstituted heteroalkyl, substituted or unsubstituted cycloalkyl, substituted or unsubstituted heterocycloalkyl, substituted or unsubstituted alkenyl, substituted or unsubstituted alkynyl, substituted or unsubstituted heteroalkenyl, substituted or unsubstituted heteroalkynyl, substituted or unsubstituted aryl, and substituted or unsubstituted heteroaryl, or isotopic substituents thereof.

In another preferred embodiment, A6, A7, A8, and A9 are each independently selected from: CR, N, wherein R is selected from: alkyl, haloalkyl, H, cycloalkyl, —COalkyl, —SO₂-alkyl, NH₂, —NHalkyl, —N(alkyl)₂.

In another preferred embodiment, A6, A7, A8, and A9 are each independently selected from: CR, N, wherein R is selected from: C₁₋₆ alkyl, H, C₃₋₆ cycloalkyl, —CO—C₁₋₆ alkyl, —SO₂-C₁₋₆ alkyl, NH₂, —NHC₁₋₆ alkyl, —N(C₁₋₆ alkyl)₂, halogenated C₁₋₆ alkyl.

In another preferred embodiment, A6 and A9 are each independently selected from: CR, N, wherein R is selected from: alkyl, haloalkyl, H, cycloalkyl;

A7 and A8 are each independently selected from: CR, N, wherein R is selected from: H, —CO-alkyl, —SO₂-alkyl, NH₂, —NHalkyl, —N(alkyl)₂, haloalkyl.

In another preferred embodiment, A6 and A9 are each independently selected from: CR, N, wherein R is selected from: C₁₋₆ alkyl, halogenated C₁₋₆ alkyl, H, C₃₋₆ cycloalkyl;
A7 and A8 are each independently selected from: CR, N, wherein R is selected from: H, —CO—C₁₋₆ alkyl, —SO₂-C₁₋₆ alkyl, NH₂, —NHC₁₋₆ alkyl, —N(C₁₋₆ alkyl)₂, halogenated C₁₋₆ alkyl.

In another preferred embodiment, A6 and A9 are each independently selected from: CR, N, wherein R is selected from: methyl, ethyl, n-propyl, isopropyl, trifluoromethyl, difluoromethyl, monofluoromethyl, fluoroisobutyl, H, cyclopropyl, cyclobutyl, cyclohexyl;
A7 and A8 are each independently selected from: CR, N, wherein R is selected from: H, acetyl, propionyl, butyryl, methylsulfonyl, ethylsulfonyl, NH₂, —NHCH₃, —NH CH₂CH₃, N(CH₃)(CH₂CH₃), N(CH₂CH₃)₂, trifluoromethyl, difluoromethyl, monofluoromethyl, and fluoroisobutyl.

In one embodiment of the first aspect, the aromatic substituent-containing (hetero)aryl imidazole compound is selected from Compound 1-Compound 105.

In the second aspect, the present application provides use of the aromatic substituent-containing (hetero)aryl imidazole compound according to the first aspect, the stereoisomer, hydrate, solvate, pharmaceutically acceptable salt or prodrug thereof as soluble epoxide hydrolase inhibitor.

In the third aspect, the present application provides a pharmaceutical composition, which includes the aromatic substituent-containing (hetero)aryl imidazole compound according to the first aspect, the stereoisomer, hydrate, solvate, pharmaceutically acceptable salt or prodrug thereof, and a pharmaceutically acceptable carrier, adjuvant or excipient.

In the fourth aspect, the present application provides a kit comprising the aromatic substituent-containing (hetero)aryl imidazole compound according to the first aspect, the stereoisomer, hydrate, solvate, pharmaceutically acceptable salt or prodrug thereof, or the pharmaceutical composition as described above.

In the fifth aspect, the present application provides use of the aromatic substituent-containing (hetero)aryl imidazole compound according to the first aspect, the stereoisomer, hydrate, solvate, pharmaceutically acceptable salt or prodrug thereof in the preparation of a medicament for treating sEH-related disease.

In the sixth aspect, the present application provides a method for preparing the aromatic substituent-containing (hetero)aryl imidazole compound according to the first aspect, characterized in that the method comprises the following steps:
S1: reacting a halogenated o-phenylenediamine precursor with a benzaldehyde precursor to obtain a (hetero)aryl imidazole intermediate;
S2: reacting the (hetero)aryl imidazole intermediate with a boronic acid precursor to obtain the aromatic substituent-containing (hetero)aryl imidazole compound;
wherein, the halogenated o-phenylenediamine precursor has a structure represented by the following general formula (1):

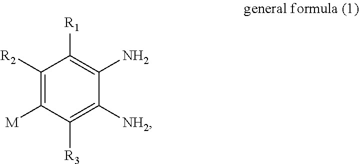

wherein, the benzaldehyde precursor has a structure represented by the following general formula (2):

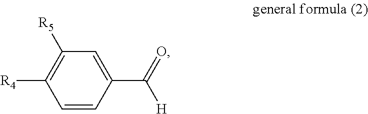

wherein, the (hetero)aryl imidazole intermediate has a structure represented by the following general formula (3):

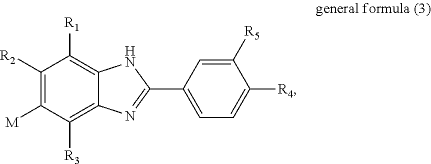

wherein, the boric acid precursor is Ar1-B(OH) 2, Ar1-BF₄K or has a structure represented by the following general formula (4):

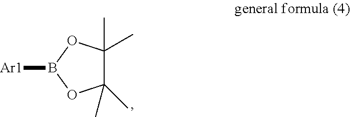

wherein R₁, R₂, R₃, R₄, R₅ and Ar1 are defined as above;
wherein, M represents a halogen.

Compared with the prior art, the beneficial effect of the present application lies in that in the (hetero)aryl imidazole compounds containing aromatic substituents described herein, at least one unsaturated carbon atom in the (hetero)aromatic ring of the (hetero)aryl imidazole structure is directly connected to an unsaturated carbon atom of the first aromatic substituent, and the carbon atom adjacent to two N atoms in the imidazole ring of the (hetero)aryl imidazole structure is directly connected to an unsaturated carbon atom of the second aromatic substituent, so that the (hetero)aryl imidazole compounds containing an aromatic substituent exhibit extremely high inhibitory activity against soluble epoxide hydrolases, and the IC₅₀ value is at the nanomolar level or even lower.

### DETAILED DESCRIPTION OF THE INVENTION

### Definition of Terms

It is to be understood that the terms used herein are for the purpose of describing particular embodiments and are not intended to be limiting.

In the present invention, unless otherwise specified, the terms used have the general meanings well known to those skilled in the art.

In the present invention, the term “C₁-C₆” means that there is 1, 2, 3, 4, 5 or 6 carbon atoms, “C₁-C₈” means that there is 1, 2, 3, 4, 5, 6, 7 or 8 carbon atoms, and so on. “3-8 membered” means that there are 3, 4, 5, 6, 7 or 8 ring atoms, and so forth.

The term “acyl” as used herein represents hydrogen or alkyl, as defined herein, attached to the parent molecular group through a carbonyl, as defined herein, including but not limited to formyl (i.e., carboxaldehyde group), acetyl, trifluoroacetyl, propionyl, and butyryl. Exemplary unsubstituted acyl includes from 1 to 6, from 1 to 11, or from 1 to 21 carbon atoms.

As used herein, the term “alkyl” refers to a branched or straight-chain monovalent saturated aliphatic hydrocarbon group having 1 to 20 carbon atoms (eg, 1 to 16 carbon atoms, 1 to 10 carbon atoms, or 1 to 6 carbon atoms). An alkylene group is a divalent alkyl.

The term “alkenyl” as used herein, alone or in combination with other groups, refers to a straight or branched chain hydrocarbon residue having a carbon-carbon double bond and having 2-20 carbon atoms (e.g., 2-16 carbon atoms, 2-10 carbon atoms, 2-6, or 2 carbon atoms).

The term “alkynyl” as used herein, alone or in combination with other groups, refers to a straight or branched chain hydrocarbon residue having a carbon-carbon triple bond and having 2-20 carbon atoms (e.g., 2-16 carbon atoms, 2-10 carbon atoms, 2-6, or 2 carbon atoms).

The term “amino” as used herein represents —N(RN₁)₂, wherein each RN₁ is independently H, N(RN₂)₂, SO₂ORN₂, SO₂RN₂, SORN₂, N-protecting group, alkyl, alkoxy, aryl, heteroaryl, arylalkyl, cycloalkyl, heterocycloalkyl, acyl (e.g., acetyl, trifluoroacetyl, or other groups described herein), wherein each of these described RN₁ groups may be optionally further substituted; or two RN₁ are combined to form an alkylene or heteroalkylene, and wherein each RN₂ is independently H, alkyl, or aryl. The amino of the present invention may be an unsubstituted amino (ie, —NH₂) or a substituted amino (ie, —N(RN₁)₂).

The term “aryl” as used herein refers to an aromatic monocyclic or polycyclic ring system, wherein all of the ring constituent atoms are carbon. For example, phenyl and naphthyl. The hydrogen atoms of the ring-forming carbon atoms may be further substituted by one or more different substituents. Furthermore, two or more adjacent substituents may be interconnected to form a non-aromatic fused, bridged and/or spirocyclic system, and the fused, bridged and/or spirocyclic system may contain one or more unsaturated structures (including but not limited to olefins, alkynes, etc.), and one or more carbon atoms constituting the ring system may be optionally substituted by atoms selected from nitrogen, oxygen and sulfur. Examples of such groups include, but are not limited to, 1,2,3,4-tetrahydronaphthyl, 1,2-dihydronaphthyl, indanyl, 1H-indenyl,

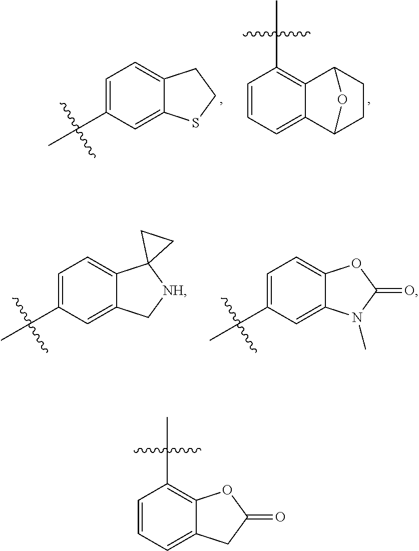

and the like.

As used herein, the term “aromatic substituent” refers to an unsaturated substituent having aromaticity and consisting of 5 to 14 atoms, and the atoms forming the ring may include one or more of carbon atoms, nitrogen atoms, oxygen atoms and sulfur atoms. The aromatic substituents may be further substituted with various substituents. Furthermore, two or more adjacent substituents may be interconnected to form a non-aromatic fused, bridged and/or spirocyclic system, and the fused, bridged and/or spirocyclic system may contain one or more unsaturated structures (including but not limited to olefins, alkynes, etc.), and one or more carbon atoms constituting the ring system may be optionally substituted by atoms selected from nitrogen, oxygen and sulfur.

The term “arylalkyl” as used herein represents an alkyl substituted with an aryl. Exemplary unsubstituted arylalkyls include from 7 to 30 carbons (including, but not limited to, from 7 to 16 or from 7 to 20 carbons, such as C₆₋₁₀ aryl C₁₋₆ alkyl, C₆₋₁₀ aryl C₁₋₁₀ alkyl, or C₆₋₁₀ aryl C₁₋₂₀ alkyl), for example, benzyl and phenethyl. In some embodiments, the alkyl and aryl can each be further substituted with 1, 2, 3, or 4 substituents as defined herein for the respective groups.

The term “cyano” as used herein represents a —CN group.

As used herein, the term “carbocyclic group” refers to a C₃₋₁₄ monocyclic, bicyclic, or tricyclic structure, wherein the ring structure as a whole is not aromatic but may contain one or more unsaturated structures, including but not limited to, alkenes, alkynes, etc. The ring may be further substituted with one or more substituents. When there are two or more rings in the carbocyclic group, the rings may further form a fused ring, a bridged ring, a spiro ring or any combination thereof, and the heteroatom (if any) may be located in any suitable position.

The term “cycloalkyl” as used herein refers to a saturated, non-aromatic monovalent mono- or poly-carbocyclic radical having 3 to 14 carbon atoms. The term is further exemplified by groups such as cyclopropyl, cyclobutyl, cyclopentyl, cyclohexyl, cycloheptyl, norbornyl and adamantyl.

As used herein, the term “halogen” refers to a fluorine (fluoro), chlorine (chloro), bromine (bromo), or iodine (iodo) group.

The term “heteroalkyl” as used herein refers to an alkyl as defined herein, in which one or more of the constituent carbon atoms have been substituted by nitrogen, oxygen, or sulfur. In some embodiments, the heteroalkyl can be further substituted with 1, 2, 3, or 4 substituents as described herein for an alkyl. “Alkoxy” represents all branched and straight chain isomers having the specified number of carbon atoms, wherein the terminal hydrogen atom is substituted by an oxygen atom. Representative examples include, but are not limited to, methoxy, ethoxy, propoxy, isopropoxy.

The term “heteroalkenyl” as used herein refers to an alkenyl group as defined herein wherein one or more of the non-olefinic constituent carbon atoms have been substituted by nitrogen, oxygen, or sulfur. In some embodiments, the heteroalkenyl can be further substituted with 1, 2, 3, or 4 substituents as described herein for alkenyl.

The term “heteroalkynyl” as used herein refers to an alkynyl group as defined herein in which one or more of the non-alkyne constituent carbon atoms has been substituted by nitrogen, oxygen, or sulfur. In some embodiments, the heteroalkynyl group can be further substituted with 1, 2, 3, or 4 substituents as described herein for alkynyl groups.

The term “heteroaryl” as used herein refers to an aromatic monocyclic or polycyclic system, wherein the ring constituent atoms (preferably 5-12) contain, in addition to carbon atoms, at least one (e.g., 1, 2, 3 or 4) atom(s) selected from nitrogen, oxygen, and sulfur, and the nitrogen atom and sulfur atom in the ring may also be in the form of suitable oxides thereof, including but not limited to nitrogen oxides, —S(O)—, and the like. The hydrogen atoms of the ring atoms may be further substituted with one or more different substituents. Furthermore, two or more adjacent substituents may be interconnected to form a non-aromatic fused, bridged and/or spirocyclic system, and the fused, bridged and/or spirocyclic system may contain one or more unsaturated structures (including but not limited to olefins, alkynes, etc.), and one or more carbon atoms constituting the ring system may be optionally replaced by atoms selected from nitrogen, oxygen and sulfur. Examples of heteroaryls include, but are not limited to, pyridyl, pyrimidinyl, pyrazolyl, benzoxazolyl, benzimidazolyl, benzothiazolyl, pyridoimidazolyl, imidazolyl, oxazolyl, thiazolyl,

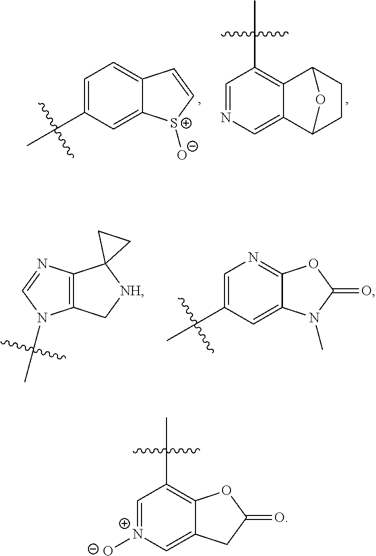

The term “heteroarylalkyl” as used herein represents an alkyl substituted with a heteroaryl. Exemplary unsubstituted heteroarylalkyls include, but are not limited to, from 7 to 30 carbons (such as from 7 to 16 or from 7 to 20 carbons, such as C₂₋₉ heteroarylC₁₋₆ alkyl, C₂₋₉ heteroarylC₁₋₁₀ alkyl, or C₂₋₉ heteroarylC₁₋₂₀ alkyl). In some embodiments, the alkyl and heteroaryls can each be further substituted with 1, 2, 3, or 4 substituents as defined herein for the respective groups.

The term “heterocyclyl” as used herein refers to a monocyclic or polycyclic non-aromatic system containing at least one (e.g., 1, 2, 3 or 4) ring heteroatoms selected from N, O or S, which may contain one or more unsaturated structures, including but not limited to, alkenes, alkynes, etc. The nitrogen atom and sulfur atom in the ring may also be in the form of suitable oxides, including but not limited to nitrogen oxides, —S(O)—, —S(O)₂—, etc. The ring may be further substituted with one or more substituents. When there are two or more rings in the heterocyclyl, the rings may further form a fused ring, a bridged ring, a spiro ring or any combination thereof, and the heteroatom may be located in any suitable position. Examples of heterocyclyls include, but are not limited to, morpholinyl, thiomorpholinyl, furanyl, piperazinyl, piperidinyl, pyranyl, pyrrolidinyl, tetrahydropyranyl, tetrahydrofuranyl, 1,3-dioxanyl, decahydroisoquinolinyl,

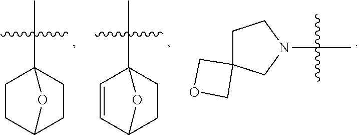

The term “heterocyclylalkyl” as used herein represents an alkyl substituted with a heterocyclyl group. Exemplary unsubstituted heterocyclylalkyls include, but are not limited to, from 7 to 30 carbons (e.g., from 7 to 16 or from 7 to 20 carbons, such as C₂₋₉ heterocyclyl C₁₋₆ alkyl, C₂₋₉ heterocyclyl C₁₋₁₀ alkyl, or C₂₋₉ heterocyclyl C₁₋₂₀ alkyl). In some embodiments, the alkyl and heterocyclyl groups can each be further substituted with 1, 2, 3, or 4 substituents as defined herein for the respective groups.

The term “hydroxyl” as used herein represents an —OH group.

The term “thiol” as used herein represents a —SH group.

Alkyl, alkenyl, alkynyl, heteroalkyl, heteroalkenyl, heteroalkynyl, carbocyclyl (eg, cycloalkyl), aryl, heteroaryl, and heterocyclyl groups can be substituted or unsubstituted. When substituted, typically 1 to 4 substituents will be present unless otherwise specified. Substituents include, but are not limited to, aryl (such as substituted and unsubstituted phenyl), carbocyclyl (such as substituted and unsubstituted cycloalkyl), halogen (such as fluoro), hydroxy, heteroalkyl (such as substituted and unsubstituted methoxy, ethoxy, or thioalkoxy), heteroaryl, heterocyclyl, amino (such as NH₂ or mono- or dialkylamino), azido, cyano, nitro, thiol, sulfonyl, sulfuryl, alkoxycarbonyl, methylene, oxo (—O), and the like. Aryl, carbocyclyl (eg, cycloalkyl), heteroaryl, and heterocyclyl may also be substituted with alkyl (unsubstituted and substituted such as arylalkyl (eg, substituted and unsubstituted benzyl)).

In practicing the methods of the present invention, an “effective amount” of any one of the compounds of the present invention or any combination of compounds of the present invention or pharmaceutically acceptable salts thereof is administered by any common and acceptable method known in the art (alone or in combination).

As used herein, the term “epoxyeicosatrienoic acid” refers to a signaling molecule formed by the action of cytochrome P450 cyclooxygenase on 20-carbon essential fatty acids, such as arachidonic acid. “Dihydroxyeicosatrienoic acid” is the corresponding vicinal diol produced by hydrolysis of epoxyeicosatrienoic acid, for example, by a soluble epoxide hydrolase. As used herein, the term “increasing the level of epoxyeicosatrienoic acid” means that after administration of the compound of the present invention, the total level of epoxyeicosatrienoic acid in a subject is increased compared to the total level of epoxyeicosatrienoic acid in the subject before administration. As used herein, the term “reducing the level of dihydroxyeicosatrienoic acid” means that after administration of the compound of the present invention, the total level of dihydroxyeicosatrienoic acid in a subject is increased compared to the total level of dihydroxyeicosatrienoic acid in the subject before administration.

As used herein, the term “inhibiting soluble epoxide hydrolase” refers to inhibiting the enzymatic activity of soluble epoxide hydrolase, and half inhibition concentration IC₅₀ is less than 10 μM (such as less than 5 μM, less than 1 μM, less than 500 nM, less than 100 nM, less than 10 nM, etc.). The enzymatic activity of the soluble epoxide hydrolase can be determined using any method known in the art, for example, the activity can be determined using an assay utilizing (3-phenyl-oxiranyl)-acetic acid cyano-(6-methoxy-naphthalen-2-yl)-methyl ester (PHOME) as a substrate (Analytical Biochemistry 2005, 343, 66-75). In this particular assay, hydrolysis of PHOME by epoxide hydrolases produces highly fluorescent 6-methoxy-2-naphthaldehyde, the concentration of which can be analytically determined using an excitation wavelength of 330 nm and an emission wavelength of 465 nm.

As used herein, the term “pharmaceutical composition” refers to a composition containing a compound described herein formulated with a pharmaceutically acceptable excipient and approved by a governmental regulatory agency for manufacture or sale as part of a therapeutic regimen for treating a disease in a mammal. The pharmaceutical composition can be formulated, for example, in unit dosage form for oral administration (e.g., tablets, capsules, caplets, soft gels, or syrups); for topical administration (e.g., as a cream, gel, lotion, or ointment); for intravenous administration (e.g., as a particulate-free plug and a sterile solution in a solvent system suitable for intravenous use); or in any other pharmaceutically acceptable formulation. As used herein, “pharmaceutically acceptable excipient” refers to any ingredient other than a compound described herein (eg, a vehicle capable of suspending or dissolving an active compound) and having the property of being substantially non-toxic and non-inflammatory in patients. Excipients may include, for example, anti-adherents, antioxidants, binders, coatings, tableting aids, disintegrants, dyes (colorants), emollients, emulsifiers, fillers (diluents), film formers or coatings, flavoring agents, fragrance enhancers, glidants (flow enhancers), lubricants, preservatives, printing inks, adsorbents, suspending or dispersing agents, sweeteners, and water for hydration. Exemplary excipients include, but are not limited to, butylated hydroxytoluene (BHT), calcium carbonate, calcium phosphate (dibasic), calcium stearate, cross-linked carboxymethylcellulose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methylparaben, microcrystalline cellulose, polyethylene glycol, polyvinyl pyrrolidone, povidone, pregelatinized starch, propylparaben, retinyl palmitate, shellac, silicon dioxide, sodium carboxymethylcellulose, sodium citrate, sodium starch glycolate, sorbitol, starch (corn), stearic acid, sucrose, talc, titanium dioxide, vitamin A, vitamin E, vitamin C, and xylitol.

As used herein, the term “stereoisomer” refers to isomers produced by different spatial arrangements of atoms in molecules. It can be divided into cis-trans isomers and enantiomers, or into two major categories: enantiomers and diastereomers. Stereoisomers caused by rotation about single bonds are called conformational stereoisomers, sometimes also called rotamers. Stereoisomers caused by bond length, bond angle, double bonds in molecules, rings, etc. are called configuration stereo-isomers, and configuration isomers are divided into two categories. Among them, the isomers caused by the inability of double bonds or single bonds of ring carbon atoms to rotate freely are called geometric isomers, also called cis-trans isomers, which are divided into two configurations: Z and E. For example: cis-2-butene and trans-2-butene are a pair of geometric isomers. Stereoisomers with different optical properties caused by the lack of anti-axis symmetry in the molecule are called optical isomers, which are divided into R and S configurations. In the present invention, the “stereoisomer” mentioned herein, unless otherwise specified, may be understood to include one or more of the above-mentioned enantiomers, configurational isomers and conformational isomers.

As used herein, the term “pharmaceutically acceptable salt” means any pharmaceutically acceptable salt of a compound of formula (I). For example, pharmaceutically acceptable salts of any compound described herein include those that are, within the scope of sound medical judgment, suitable for use in contact with the tissues of humans and animals without excessive toxicity, irritation, allergic response, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and Pharmaceutical Salts: Properties, Selection, and Use (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the present invention may have ionizable groups so as to be prepared as pharmaceutically acceptable salts. These salts may be acid addition salts involving inorganic or organic acids, or salts may (in the case of acid forms of the compounds of the invention) be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparing appropriate salts are well known in the art. Salts can be prepared from pharmaceutically acceptable non-toxic acids and bases, including inorganic and organic acids and bases.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, heptanoate, hexanoate, hydrobromide, hydrochloride, glycerophosphate, hemisulfate, 1 hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate. Representative alkali or alkaline earth metal salts include sodium, lithium, potassium, calcium, and magnesium, as well as nontoxic ammonium, quaternary ammonium, and amine cations including, but not limited to, ammonium, tetramethylammonium, tetraethylammonium, methylamine, dimethylamine, trimethylamine, triethylamine, and ethylamine.

As used herein, the term “subject” refers to any organism to which a composition according to the invention can be administered, for example, for experimental, for diagnostic, preventive and/or therapeutic purposes. Typical subjects include any animal (eg, mammals such as mice, rats, rabbits, non-human primates, and humans). A subject may be a human or animal who seeks treatment or is in need of treatment, requires treatment, is receiving treatment, will receive treatment in the future, or is under the care of a trained professional for a specific disease or condition.

As used herein, the terms “treat” or “therapeutic” refer to both therapeutic treatment and prophylactic or preventative measures, wherein the object is to prevent or slow down (lessen) an undesirable physiological condition, disorder, or disease, or to obtain beneficial or desired clinical results. Beneficial or desired clinical results include, but are not limited to, relief of symptoms; lessening of the extent of a condition, disorder, or disease; stabilization of the condition, disorder, or disease state (i.e., not worsening); delay or slowing of the onset of the condition, disorder, or disease progression; improvement or remission of the condition, disorder, or disease state (whether partial or complete), whether detectable or undetectable; improvement in at least one measurable human physiological parameter, which parameter need not be discernible by the patient; or amelioration or improvement of a condition, disorder, or disease. Treatment involves inducing a clinically significant response without excessive levels of side effects. Treatment also includes prolonged survival as compared to expected survival if not receiving treatment.

### Compound

In a specific embodiment, the present application provides an aromatic substituent-containing (hetero)aryl imidazole compound, and a stereoisomer, hydrate, solvate, pharmaceutically acceptable salt or prodrug thereof. The aromatic substituent-containing (hetero)aryl imidazole compound has a structure shown by the following general formula IA or general formula IB:

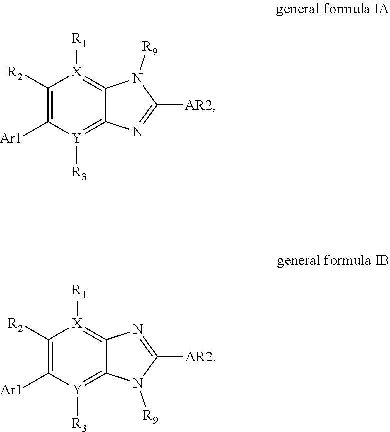

In the general formulae IA and IB, X and Y each independently represent C or N. In one specific embodiment, X and Y are both carbon atoms, or X and Y are both nitrogen atoms. In another embodiment, X is a carbon atom and Y is a nitrogen atom. In another embodiment, X is a nitrogen atom and Y is a carbon atom.

In the general formulas IA and IB, R₁, R₂ and R₃ are each independently selected from the following group: hydrogen, deuterium, tritium, halogen, substituted or unsubstituted straight-chain alkyl, substituted or unsubstituted branched-chain alkyl, substituted or unsubstituted heteroalkyl, substituted or unsubstituted cycloalkyl, substituted or unsubstituted heterocycloalkyl, cyano, trifluoromethyl, alkoxy, cycloalkyloxy, aryloxy, substituted or unsubstituted amino, substituted or unsubstituted thiol, substituted or unsubstituted sulfonyl, substituted or unsubstituted sulfuryl, substituted or unsubstituted alkenyl, substituted or unsubstituted alkynyl, substituted or unsubstituted heteroalkenyl, substituted or unsubstituted heteroalkynyl, substituted or unsubstituted aryl, and substituted or unsubstituted heteroaryl, or isotopic substituents thereof. It should be noted that when X is a nitrogen atom, there is no R₁ group. Similarly, when Y is a nitrogen atom, R₃ is not present. In a preferred embodiment, R₁, R₂ and R₃ are each independently selected from a group consisting of hydrogen, fluorine and chlorine. Preferably, R₂ is fluorine, and R₁ and R₃ are hydrogen. Preferably, R₁ and R₂ are fluorine, and R₃ is hydrogen.

In the general formula IA and IB, R₉ is selected from the following group: hydrogen, deuterium, tritium, halogen, substituted or unsubstituted straight chain alkyl, substituted or unsubstituted branched chain alkyl, substituted or unsubstituted heteroalkyl, substituted or unsubstituted cycloalkyl, substituted or unsubstituted heterocycloalkyl, cyano, trifluoromethyl, alkoxy, cycloalkyloxy, aryloxy, substituted or unsubstituted amino, substituted or unsubstituted thiol, substituted or unsubstituted sulfonyl, substituted or unsubstituted sulfuryl, substituted or unsubstituted alkenyl, substituted or unsubstituted alkynyl, substituted or unsubstituted heteroalkenyl, substituted or unsubstituted heteroalkynyl, substituted or unsubstituted aryl, and substituted or unsubstituted heteroaryl, or isotopic substituents thereof, or a prodrug group of the parent structure.

In the general formulae IA and IB, Ar1 and Ar2 are each independently selected from a group consisting of substituted or unsubstituted aryl, and substituted or unsubstituted heteroaryl. At least one unsaturated carbon atom in the (hetero)aromatic ring of the (hetero)aryl imidazole structure is directly connected to an unsaturated carbon atom of Ar1, and the carbon atom adjacent to two N atoms in the imidazole ring of the (hetero)aryl imidazole structure is directly connected to an unsaturated carbon atom of Ar2.

In a specific embodiment, the aromatic substituent-containing (hetero)aryl imidazole compound has a structure represented by the following general formula IIA or IIB:

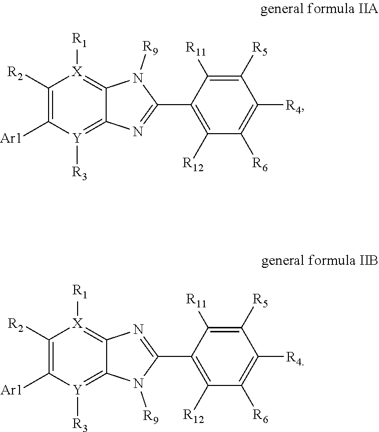

In the general formulas IIA and IIB, R₄, R₅, R₆, R₁₁ and R₁₂ are each independently selected from the following group: hydrogen, deuterium, tritium, halogen, substituted or unsubstituted straight-chain alkyl, substituted or unsubstituted branched-chain alkyl, substituted or unsubstituted heteroalkyl, substituted or unsubstituted cycloalkyl, substituted or unsubstituted heterocycloalkyl, cyano, trifluoromethyl, alkoxy, cycloalkyloxy, aryloxy, substituted or unsubstituted amino, substituted or unsubstituted thiol, substituted or unsubstituted sulfonyl, substituted or unsubstituted sulfuryl, substituted or unsubstituted alkenyl, substituted or unsubstituted alkynyl, substituted or unsubstituted heteroalkenyl, substituted or unsubstituted heteroalkynyl, substituted or unsubstituted aryl, and substituted or unsubstituted heteroaryl, or isotopic substituents thereof.

In a specific embodiment, R₁ and R₂ are connected to form a five-membered ring, a six-membered ring, a seven-membered ring or an eight-membered ring. In another embodiment, R₄ and R₅ are linked to form a five-membered ring, a six-membered ring, a seven-membered ring, or an eight-membered ring. In another embodiment, alternatively or additionally, R₄ and R₆ are linked to form a five-membered ring, a six-membered ring, a seven-membered ring, or an eight-membered ring.

In one embodiment, Ar1 has a structure represented by the following general formula III:

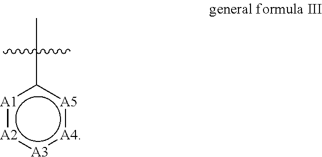

In the general formula III, A1, A2, A3, A4 and A5 are each independently selected from the following group: CR, N, N—R, S and O, wherein R is each independently selected from the following group: hydrogen, deuterium, tritium, halogen, cyano, trifluoromethyl, alkoxy, cycloalkyloxy, aryloxy, substituted or unsubstituted amino, substituted or unsubstituted thiol, substituted or unsubstituted sulfonyl, substituted or unsubstituted sulfuryl, substituted or unsubstituted straight-chain alkyl, substituted or unsubstituted branched-chain alkyl, substituted or unsubstituted heteroalkyl, substituted or unsubstituted cycloalkyl, substituted or unsubstituted heterocycloalkyl, substituted or unsubstituted alkenyl, substituted or unsubstituted alkynyl, substituted or unsubstituted heteroalkenyl, substituted or unsubstituted heteroalkynyl, substituted or unsubstituted aryl, and substituted or unsubstituted heteroaryl, or isotopic substituents thereof.

In a preferred embodiment, Ar1 has a structure represented by the following general formula IV:

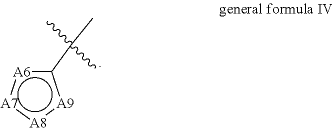

In the general formula IV, A6, A7, A8, A8 and A9 are each independently selected from the following group: CR, N, N—R, S and O, wherein R is each independently selected from the following group: hydrogen, deuterium, tritium, halogen, cyano, trifluoromethyl, alkoxy, cycloalkyloxy, aryloxy, substituted or unsubstituted amino, substituted or unsubstituted thiol, substituted or unsubstituted sulfonyl, substituted or unsubstituted sulfuryl, substituted or unsubstituted straight-chain alkyl, substituted or unsubstituted branched-chain alkyl, substituted or unsubstituted heteroalkyl, substituted or unsubstituted cycloalkyl, substituted or unsubstituted heterocycloalkyl, substituted or unsubstituted alkenyl, substituted or unsubstituted alkynyl, substituted or unsubstituted heteroalkenyl, substituted or unsubstituted heteroalkynyl, substituted or unsubstituted aryl, and substituted or unsubstituted heteroaryl, or isotopic substituents thereof.

In a particularly preferred embodiment, the aromatic substituent-containing (hetero)aryl imidazole compound is selected from Compound 1-Compound 105.

As used herein, the terms alkyl, alkenyl and alkynyl include straight or branched chain monovalent substituents. Examples thereof include methyl, ethyl, isobutyl, 3-butynyl and the like. Examples of groups that can be used in the compounds and methods described herein can include C₁-C₂₀ alkyl, C₂-C₂₀ alkenyl, and C₂-C₂₀ alkynyl. The terms heteroalkyl, heteroalkenyl, and heteroalkynyl are defined similarly to alkyl, alkenyl, and alkynyl, but their backbones may include O, S, N heteroatoms, or combinations thereof.

The terms cycloalkyl, cycloalkenyl, and cycloalkynyl may include cycloalkyls having one ring or multiple fused rings. Examples thereof may include cyclohexyl, cyclopentylethyl and adamantyl group. The terms heterocycloalkyl, heterocycloalkenyl and heterocycloalkynyl are defined similarly to cycloalkyl, cycloalkenyl and cycloalkynyl, but their backbones may include O, S, N heteroatoms or combinations thereof.

Aryl molecules may include cyclic hydrocarbons that may include one or more flat groups of carbon atoms (usually six) linked together by an equal number of delocalized electrons. It's as if they consist of alternating single and double bonds. An example of an aryl molecule is a benzene ring. The carbon atoms forming the ring of the heteroaryl molecule may be substituted by O, N or S. When a heteroatom is introduced, a group of five atoms (eg, four carbon atoms and one heteroatom) can form an aromatic system. Examples of heteroaryl molecules may include furan, pyrrole, thiophene, imidazole, oxazole, pyridine, and piperazine. Aryl and heteroaryl molecules may also include additional fused rings, for example benzofuran, indole, benzothiophene, naphthalene, anthracene, and quinoline. Unless otherwise specified, aryl and heteroaryls may be attached at any position on the aromatic ring.

The alkoxy, cycloalkoxy, amino, alkyl, alkenyl, alkynyl, heteroalkyl, heteroalkenyl, heteroalkynyl, heteroaryl, cycloalkyl, or heterocycloalkyls described herein may be substituted or unsubstituted. As used herein, the term “substituted” refers to the addition of alkoxy, cycloalkoxy, aryloxy, amino, alkyl, alkenyl, alkynyl, alkyl, heteroalkyl, heteroalkenyl, heteroalkynyl, heteroaryl, cycloalkyl or heterocycloalkyls to the backbone of alkoxy, cycloalkoxy, amino, alkyl, alkenyl, alkynyl, heteroalkyl, heteroalkenyl, heteroalkynyl, heteroaryl, cycloalkyl or heterocycloalkyl. For example, a hydrogen atom is replaced by one of these groups. Examples of substituents include, but are not limited to, hydroxyl, halogen (eg, F, Cl, Br, or I), and carbonyl. In contrast, the term “unsubstituted” means that the alkoxy, cycloalkoxy, amino, alkyl, alkenyl, alkynyl, heteroalkyl, heteroalkenyl, heteroalkynyl, heteroaryl, cycloalkyl or heterocycloalkyls are all replaced by hydrogen atoms.

### Preparation Method of Compound

The (hetero)aryl imidazole compounds containing an aromatic substituent described herein can be prepared in various ways and synthesized via different synthetic routes. The (hetero)aryl imidazole compounds containing an aromatic substituent described herein can be synthesized using mass-produced raw materials, and those skilled in the art can also obtain the optimal reaction conditions based on the actual reaction reagents and solvents used.

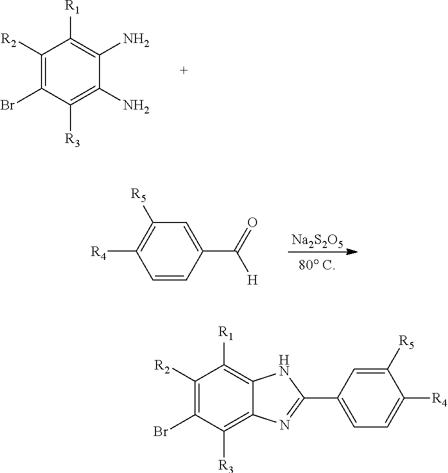

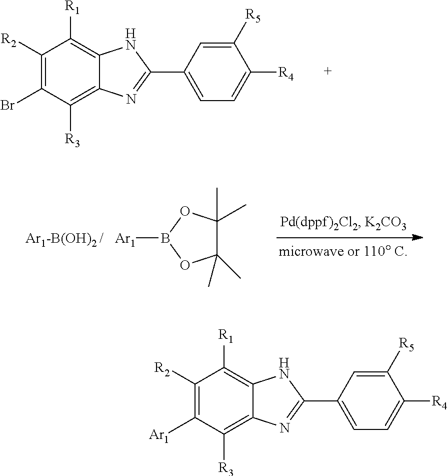

As an example, a general route for synthesizing the (hetero)aryl imidazole compounds described herein containing an aromatic substituent is shown above. The method may include the following steps:
S1: reacting a halogenated o-phenylenediamine precursor with a corresponding aldehyde precursor to obtain a (hetero)aryl imidazole intermediate;
S2: reacting the (hetero)aryl imidazole intermediate with a boric acid (ester) precursor to obtain the aromatic substituent-containing (hetero)aryl imidazole compound.

Wherein, the halogenated o-phenylenediamine precursor has a structure represented by the following general formula (1):

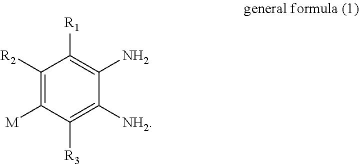

Wherein, the aldehyde precursor has a structure represented by the following general formula (2):

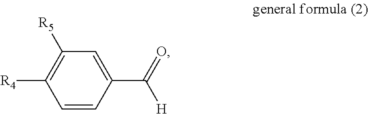

Wherein, the (hetero)aryl imidazole intermediate has a structure represented by the following general formula (3):

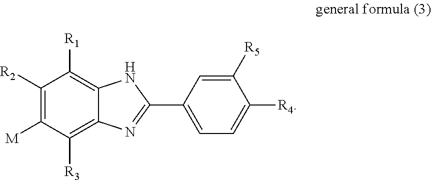

Wherein, the boric acid precursor is Ar1-B(OH)₂, Ar1-BF₄K or has a structure represented by the following general formula (4):

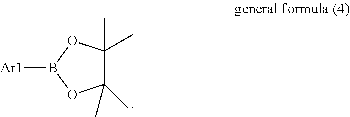

R₁, R₂, R₃, R₄, R₅ and Ar1 are defined as above. M represents halogen.

### Pharmaceutical Composition

The aromatic substituent-containing (hetero)aryl imidazole compound or a derivative thereof, a stereoisomer, a hydrate, a solvate, a pharmaceutically acceptable salt or a prodrug thereof described herein can be provided in the form of a pharmaceutical composition. The pharmaceutical composition may include a therapeutically effective amount of an aromatic substituent-containing (hetero)aryl imidazole compound and a pharmaceutically acceptable auxiliary material, carrier or excipient. Depending on the intended mode of injection, the pharmaceutical composition may be a solid, semisolid or liquid preparation. Those skilled in the art can select auxiliary materials, carriers or excipients, as well as the dosage form of the pharmaceutical composition according to actual conditions, which will not be described in detail here.

### Methods of Use

The present application also provides a method for treating or preventing a disease associated with or mediated by soluble epoxide hydrolase (sEH) in a subject. The method comprises injecting into the subject an effective amount of one or more compounds or pharmaceutical compositions described herein, or a pharmaceutically acceptable salt or prodrug thereof. When used in the methods, the term “effective amount” refers to an amount of a compound that achieves the desired biological effect. For example, an effective amount may be the concentration of the compound that inhibits sEH in vitro.

The (hetero)aryl imidazole compounds containing an aromatic substituent or pharmaceutical compositions described herein can be used to treat sEH-related or mediated disease in humans and animals. The sEH-related or mediated diseases include, but are not limited to, neurodegenerative diseases, inflammation, hypertension, etc. In another preferred embodiment, the sEH-related or mediated disease is selected from the following group: Alzheimer's disease, Parkinson's disease, Huntington's disease, dementia, cerebral ischemia, epilepsy, traumatic brain injury, stroke, Alexander disease, Alpers disease, amyotrophic lateral sclerosis, ataxia telangiectasia, Batten disease, Canavan disease, Cockayne syndrome, cortical basal degeneration, Creutzfeldt-Jakob disease, Kennedy disease, Krabbe disease, Lewy body dementia, Machado-Joseph disease, multiple sclerosis, multiple system atrophy, Pelizaeus-Merzbacher disease, Pick's disease, primary lateral sclerosis, Refsum's disease, Sandhoff disease, Schilder's disease, Tay-Sachs disease, spinocerebellar ataxia, spinal muscular atrophy, progressive supranuclear palsy, transmissible spongiform encephalopathy, tabes dorsalis, peripheral neuropathy, neuroinflammation, asthma, chronic obstructive pulmonary disease, chronic bronchitis, cystic fibrosis, atherosclerosis, restenosis after angioplasty, coronary artery disease, rheumatoid arthritis, osteoarthritis, dermatitis, eczematous dermatitis, psoriasis disease, late and chronic solid organ rejection after transplantation, systemic lupus erythematosus, dermatomyositis, polymyositis, Sjögren's syndrome, polymyalgia rheumatica, temporal arteritis, Behcet's disease, Guillain-Barré syndrome, Wegener's granulomatosis, polyarteritis nodosa, neuralgia, vasculitis, pancreatitis, ulcerative colitis, Crohn's disease, Kaposi's sarcoma, hypertension, pulmonary hypertension, adult respiratory distress syndrome, end-stage renal disease, heart failure, renal failure, liver failure, cardiac fibrosis, renal fibrosis, pulmonary fibrosis, liver fibrosis, non-alcoholic fatty liver disease, ischemic limb disease, intermittent claudication, endothelial dysfunction, male erectile dysfunction, Raynaud's disease, diabetic vascular disease, herpes zoster, gastrointestinal diseases caused by non-steroidal anti-inflammatory drugs, hand-foot syndrome caused by chemotherapy, etc. The method of treating or preventing a sEH-related or mediated disease in a subject may further comprise administering to the subject a second compound, biomolecule or composition. The additional reagents and the (hetero)aryl imidazole compounds containing an aromatic substituent described herein may be used in combination in any order.

### Kits

The present application also provides a kit for treating a disease associated with or mediated by sEH in a subject. The kit may include one or more (hetero)aryl imidazole compounds containing an aromatic substituent or pharmaceutical compositions as described herein. A kit can further include one or more additional agents, such as anti-inflammatory drug. A kit may be an oral preparation or an intravenous injection preparation made from the aromatic substituent-containing (hetero)aryl imidazole compound or pharmaceutical composition described herein. A kit may also include instructions for use of the kit, a container and a device for injecting the compound or composition, and/or a carrier.

### Use

The present application also relates to use of the aromatic substituent-containing (hetero)aryl imidazole compound as described herein, and a stereoisomer, hydrate, solvate, pharmaceutically acceptable salt or prodrug thereof as a soluble epoxide hydrolase inhibitor. The present application also relates to use of the (hetero)aryl imidazole compounds containing aromatic substituents as described herein, and a stereoisomer, hydrate, solvate, pharmaceutically acceptable salt or prodrug thereof in the preparation of a medicament for treating sEH-related or mediated disease.

### EXAMPLE

The present application will be further described and illustrated below in conjunction with examples. Unless otherwise specified, all chemical raw materials used can be purchased from the market. Those skilled in the art will appreciate that the following examples are merely illustrative.

In the following examples, the characterization methods used are as follows.

Nuclear magnetic resonance hydrogen spectrum (1H NMR):

The nuclear magnetic resonance spectra (1H NMR) of the reaction products and intermediates were obtained on an AVANCE III HD 400/500 from Bruker, Germany. The sample preparation method was as follows: in a clean and dry glass NMR tube, completely dissolve an appropriate amount of sample in about 0.5 mL of deuterated solvent. Most compounds have good solubility in DMSO-d6, so it is used as the preferred deuterated solvent. It is easy to solidify at low room temperature and needs to be blown with a hair dryer before loading. Other suitable deuterated reagents can also be selected according to test requirements, such as CDCl₃, CD₃OD, etc. When testing at room temperature, tetramethylsilane (TMS) was used as the internal standard and the chemical shift was 0 ppm.

### Example 1

This example relates to the synthesis and characterization of (hetero)aryl imidazole compounds containing an aromatic substituent.

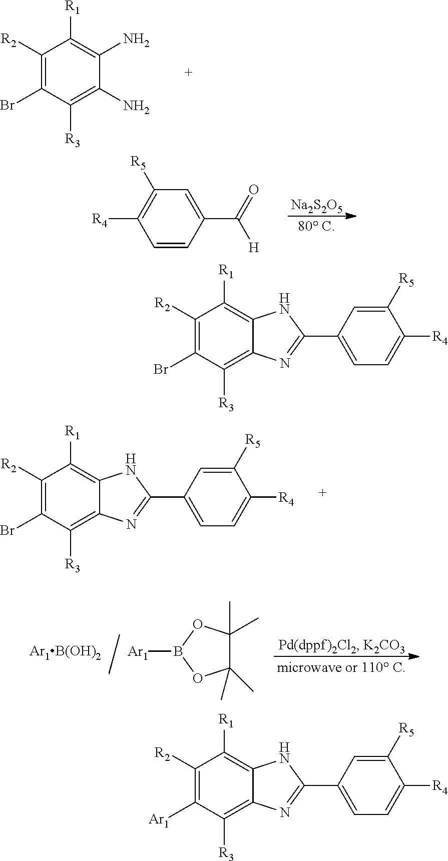

With reference to the above synthetic route, the aromatic substituent-containing (hetero)aryl imidazole compound described herein can be prepared by selecting appropriate raw materials through a two-step method. Some compounds containing special structures or functional groups can also be obtained under similar conditions by selecting appropriate reaction conditions. The general synthetic method for this two-step process is as follows.

To a solution of halobenzene-1,2-diamine precursor (2 mmol) and benzaldehyde precursor (3 mmol) in EtOH (60 mL) was added Na₂S₂O₅ solution (4 mmol in 6 mL water). The mixture was degassed and stirred at 80° C. overnight, then concentrated under reduced pressure. The residue was extracted with ethyl acetate (3×20 mL) and the combined organic layers were washed with water, then dried over Na₂SO₄. The solvent was removed under reduced pressure. The residue was purified by column chromatography to afford the corresponding (hetero)aryl imidazole intermediate.

To a flask containing the (hetero)aryl imidazole intermediate (0.1 mmol) obtained in the previous step, boronic acid precursor (0.12 mmol), potassium carbonate (0.36 mmol) and catalyst Pd(dppf)₂Cl₂ (0.015 mmol) was added 1,4-dioxane/water (2.5 mL, v/v, 9:1). The mixture was degassed three times using N₂ and then treated in a microwave (110° C., 1 hr) for 1 hour. After cooling to room temperature, the reaction mixture was filtered through celite and washed with methanol. The filtrate was concentrated and the residue was purified by preparative HPLC to obtain the corresponding target (hetero)aryl imidazole compound containing an aromatic substituent.

In this example, 105 (hetero)aryl imidazole compounds containing an aromatic substituent and having novel structures were synthesized using the general steps described above. The structures of the corresponding (hetero)aryl imidazole intermediate, boronic acid precursor and the final (hetero)aryl imidazole compound containing an aromatic substituent are shown in Table 1 below. As a specific example, the halogenated benzene-1,2-diamine precursors used in this example are all o-phenylenediamine precursors substituted with bromine at the 3-position, and R₉ is a hydrogen atom. The first step is a process well known in the art, and therefore the specific structures of the halogenated benzene-1,2-diamine precursor (also referred to as: halogenated o-diphenyl precursor) and the benzaldehyde precursor are not shown, but those skilled in the art can determine the specific structures of the halogenated benzene-1,2-diamine precursor and the benzaldehyde precursor used through the structure of the (hetero)aryl imidazole intermediate. The NMR data of each synthesized compound are shown in Table 2.

TABLE 1
Structure, chemical name, and mass spectrometry data of boronic acid (ester) intermediates, (hetero)aryl-imidazole intermediates, and (hetero)aryl-imidazole compounds containing aromatic substituents
in Example 1.

| | | | | | ESI-MS: |
|---|---|---|---|---|---|
| No. | boronic acid (ester) intermediates | (hetero)aryl-imidazole intermediates | (hetero)aryl-imidazole compounds containing aromatic substituents | chemical name | [M + 1]⁺ |
| 1 | 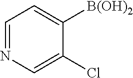 | 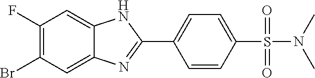 [SMILES: CN(C)S(=O)(=O)c1ccc(-c2nc3cc(Br)c(F)cc3[nH]2)cc1] | 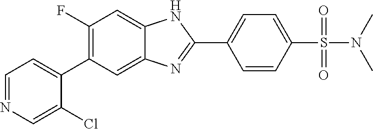 [SMILES: CN(C)S(=O)(=O)c1ccc(-c2nc3cc(-c4ccncc4Cl)c(F)cc3[nH]2)cc1] | 4-(6-(3-chloropyridin-4-yl)-5- fluoro-1H-benzo[d]imidazol- 2-yl)-N,N-dimethylbenzene- sulfonamide | 431.1 |
| 2 | 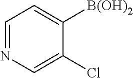 | 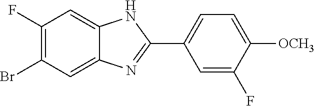 [SMILES: COc1ccc(-c2nc3cc(Br)c(F)cc3[nH]2)cc1F] | 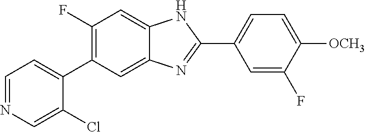 [SMILES: COc1ccc(-c2nc3cc(-c4ccncc4Cl)c(F)cc3[nH]2)cc1F] | 6-(3-chloropyridin-4-yl)-5- fluoro-2-(3-fluoro-4-meth- oxyphenyl)-1H-benzo[d] imidazole | 372.2 |
| 3 | 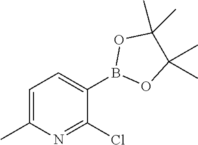 | 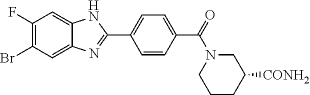 [SMILES: NC(=O)[C@@H]1CCCN(C(=O)c2ccc(-c3nc4cc(Br)c(F)cc4[nH]3)cc2)C1] | 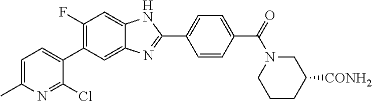 [SMILES: Cc1ccc(-c2cc3nc(-c4ccc(C(=O)N5CCC[C@@H](C(N)=O)C5)cc4)[nH]c3cc2F)c(Cl)n1] | (R)-1-(4-(6-(2-chloro-6-meth- ylpyridin-3-yl)-5-fluoro-1H- benzo[d]imidazol-2-yl)benzo- yl)piperidine-3-carboxamide | 492.2 |
| 4 | 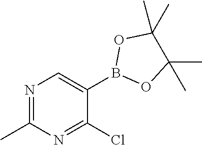 | 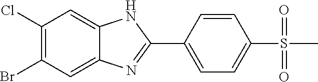 [SMILES: CS(=O)(=O)c1ccc(-c2nc3cc(Br)c(Cl)cc3[nH]2)cc1] | 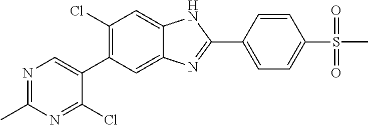 | 5-chloro-6-(4-chloro-2-meth- ylpyrimid-5-yl)-2-(4-(methyl- sulfonyl)phenyl)-1H-benzo[d] imidazole | 433.0 |
| 5 | 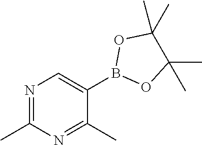 | 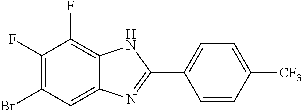 [SMILES: Fc1c(Br)cc2nc(-c3ccc(C(F)(F)F)cc3)[nH]c2c1F] | 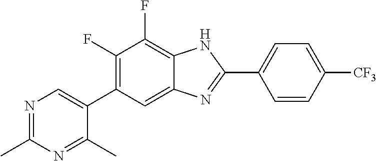 [SMILES: Cc1ncc(-c2cc3nc(-c4ccc(C(F)(F)F)cc4)[nH]c3c(F)c2F)c(C)n1] | 6-(2,4-dimethylpyrimid-5-yl)- 4,5-difluoro-2-(4-trifluoro- methyl)phenyl)-1H-benzo[d] imidazole | 405.1 |
| 6 | 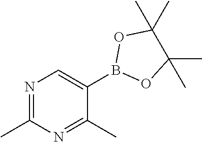 | 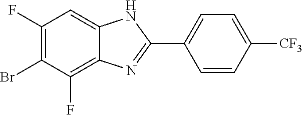 [SMILES: Fc1cc2[nH]c(-c3ccc(C(F)(F)F)cc3)nc2c(F)c1Br] | 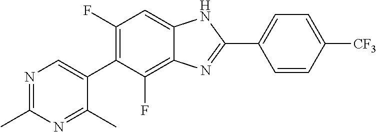 [SMILES: Cc1ncc(-c2c(F)cc3[nH]c(-c4ccc(C(F)(F)F)cc4)nc3c2F)c(C)n1] | 5,7-difluoro-6-(2-methylpyr- imid-5-yl)-2-(4-(trifluoro- methyl)phenyl)-1H-benzo[d] imidazole | 405.1 |
| 7 | 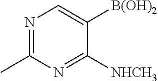 | 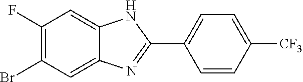 [SMILES: Fc1cc2[nH]c(-c3ccc(C(F)(F)F)cc3)nc2cc1Br] | 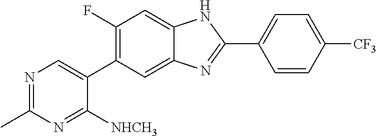 [SMILES: CNc1nc(C)ncc1-c1cc2nc(-c3ccc(C(F)(F)F)cc3)[nH]c2cc1F] | 5-(5-fluoro-2-(4-(trifluoro- methyl)phenyl)-1H-benzo[d] imidazol-6-yl)-N,2-dimethyl- pyrimidine-4-amine | 402.1 |
| 8 | 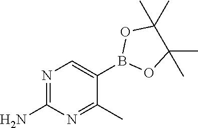 [SMILES: Cc1nc(N)ncc1B1OC(C)(C)C(C)(C)O1] | 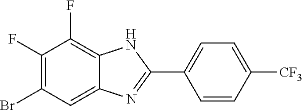 [SMILES: Fc1c(Br)cc2nc(-c3ccc(C(F)(F)F)cc3)[nH]c2c1F] | 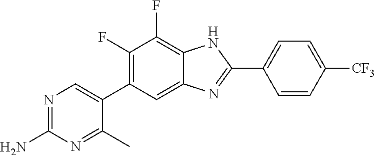 [SMILES: Cc1nc(N)ncc1-c1cc2nc(-c3ccc(C(F)(F)F)cc3)[nH]c2c(F)c1F] | 5-(4,5-difluoro-2-(4-(trifluoro- methyl)phenyl)-1H-benzo[d] imidazol-6-yl)-4-methylpyr- imidine-2-amine | 406.1 |
| 9 | 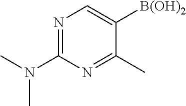 | 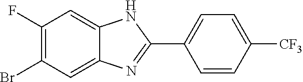 [SMILES: Fc1cc2[nH]c(-c3ccc(C(F)(F)F)cc3)nc2cc1Br] | 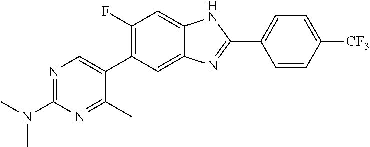 [SMILES: Cc1nc(N(C)C)ncc1-c1cc2nc(-c3ccc(C(F)(F)F)cc3)[nH]c2cc1F] | 5-(5-fluoro-2-(4-(trifluoro- methyl)phenyl)-1H-benzo[d] imidazol-6-yl)-N,N,4-trimeth- ylpyrimidine-2-amine | 416.1 |
| 10 | 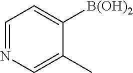 | 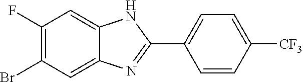 [SMILES: Fc1cc2[nH]c(-c3ccc(C(F)(F)F)cc3)nc2cc1Br] | 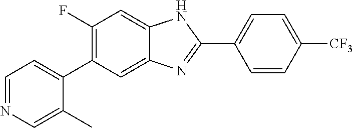 [SMILES: Cc1cnccc1-c1cc2nc(-c3ccc(C(F)(F)F)cc3)[nH]c2cc1F] | 5-(5-fluoro-2-(4-(trifluoro- methyl)phenyl)-1H-benzo[d] imidazol-6-yl)-N,N,4-trimeth- ylpyrimidine-2-amine | 372.1 |
| 11 | 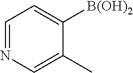 | 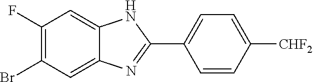 [SMILES: Fc1cc2[nH]c(-c3ccc(C(F)F)cc3)nc2cc1Br] | 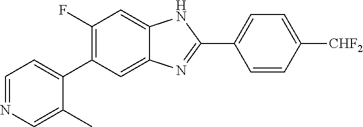 [SMILES: Cc1cnccc1-c1cc2nc(-c3ccc(C(F)F)cc3)[nH]c2cc1F] | 2-(4-(difluoromethyl)phenyl)- 5-fluoro-6-(3-methylpyridin-4- yl)-1H-benzo[d]imidazole | 354.1 |
| 12 | 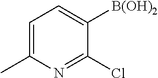 | 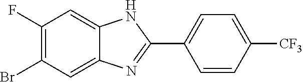 [SMILES: Fc1cc2[nH]c(-c3ccc(C(F)(F)F)cc3)nc2cc1Br] | 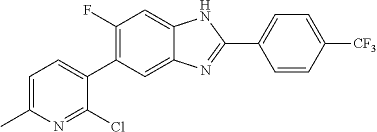 [SMILES: Cc1ccc(-c2cc3nc(-c4ccc(C(F)(F)F)cc4)[nH]c3cc2F)c(Cl)n1] | 6-(2-chloro-6-methylpyridin- 3-yl)-5-fluoro-2-(4-(trifluoro- methyl)phenyl)-1H-benzo[d] imidazole | 406.1 |
| 13 | 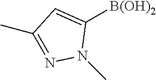 | 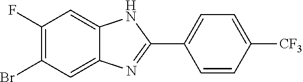 [SMILES: Fc1cc2[nH]c(-c3ccc(C(F)(F)F)cc3)nc2cc1Br] | 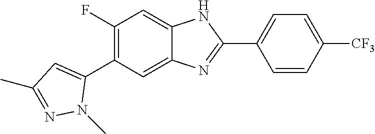 [SMILES: Cc1cc(-c2cc3nc(-c4ccc(C(F)(F)F)cc4)[nH]c3cc2F)n(C)n1] | 6-(1,3-dimethyl-1H-pyrazol- 5-yl)-5-fluoro-2-(4-(trifluoro- methyl)phenyl)-1H-benzo[d] imidazole | 375.1 |
| 14 | 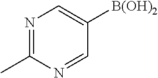 | 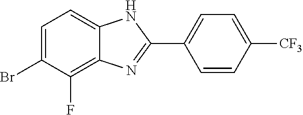 [SMILES: Fc1c(Br)ccc2[nH]c(-c3ccc(C(F)(F)F)cc3)nc12] | 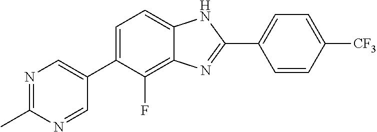 [SMILES: Cc1ncc(-c2ccc3[nH]c(-c4ccc(C(F)(F)F)cc4)nc3c2F)cn1] | 7-fluoro-6-(2-methylpyrimid- 5-yl)-2-(4-(trifluoromethyl)- phenyl)-1H-benzo[d]imidazole | 373.1 |
| 15 | 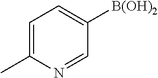 | 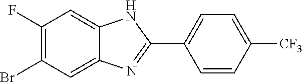 [SMILES: Fc1cc2[nH]c(-c3ccc(C(F)(F)F)cc3)nc2cc1Br] | 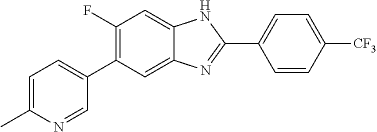 [SMILES: Cc1ccc(-c2cc3nc(-c4ccc(C(F)(F)F)cc4)[nH]c3cc2F)cn1] | 5-fluoro-6-(6-methylpyridin- 3-yl)-2-(4-(trifluoromethyl)- phenyl)-1H-benzo[d]imidazole | 372.1 |
| 16 | 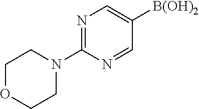 [SMILES: OB(O)c1cnc(N2CCOCC2)nc1] | 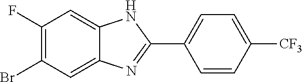 [SMILES: Fc1cc2[nH]c(-c3ccc(C(F)(F)F)cc3)nc2cc1Br] | 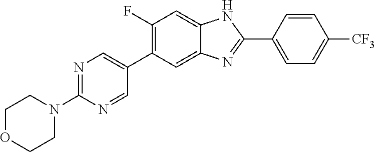 [SMILES: Fc1cc2[nH]c(-c3ccc(C(F)(F)F)cc3)nc2cc1-c1cnc(N2CCOCC2)nc1] | 4-(5-(5-fluoro-2-(4-(trifluoro- methyl)phenyl)-1H-benzo[d] imidazol-6-yl)pyrimidine-2- yl)morpholine | 444.1 |
| 17 | 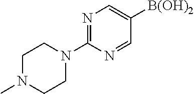 [SMILES: CN1CCN(c2ncc(B(O)O)cn2)CC1] | 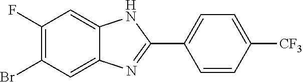 [SMILES: Fc1cc2[nH]c(-c3ccc(C(F)(F)F)cc3)nc2cc1Br] | 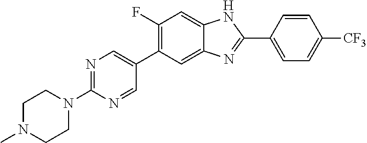 [SMILES: CN1CCN(c2ncc(-c3cc4nc(-c5ccc(C(F)(F)F)cc5)[nH]c4cc3F)cn2)CC1] | 5-fluoro-6-(2-(4-methylpiper- azine-1-yl)pyrimid-5-yl)-2-(4- (trifluoromethyl)phenyl)-1H- benzo[d]imidazole | 457.2 |
| 18 | 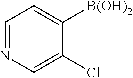 | 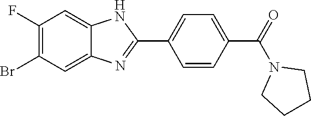 [SMILES: O=C(c1ccc(-c2nc3cc(Br)c(F)cc3[nH]2)cc1)N1CCCC1] | 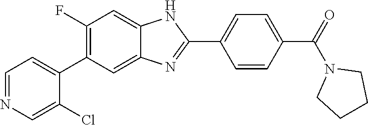 [SMILES: O=C(c1ccc(-c2nc3cc(-c4ccncc4Cl)c(F)cc3[nH]2)cc1)N1CCCC1] | (4-(6-(2-chloropyridin-3-yl)-5- fluoro-1H-benzo[d]imidazol- 2-yl)phenyl)(pyrrolidin-1-yl)- ketone | 421.1 |
| 19 | 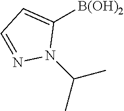 | 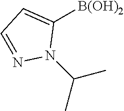 | 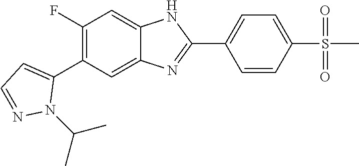 [SMILES: CC(C)n1nccc1-c1cc2nc(-c3ccc(S(C)(=O)=O)cc3)[nH]c2cc1F] | 5-fluoro-6-(1-isopropyl-1H- pyrazol-5-yl)-2-(4-(methyl- sulfonyl)phenyl)-1H-benzo[d] imidazole | 399.1 |
| 20 | 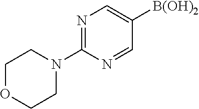 [SMILES: OB(O)c1cnc(N2CCOCC2)nc1] | 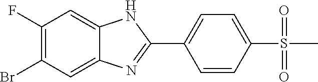 [SMILES: CS(=O)(=O)c1ccc(-c2nc3cc(Br)c(F)cc3[nH]2)cc1] | 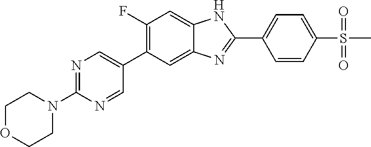 [SMILES: CS(=O)(=O)c1ccc(-c2nc3cc(-c4cnc(N5CCOCC5)nc4)c(F)cc3[nH]2)cc1] | 4-(5-(5-fluoro-2-(4-(methyl- sulfonyl)phenyl)-1H-benzo[d] imidazol-6-yl)pyrimidine-2- yl)morpholine | 454.1 |
| 21 | 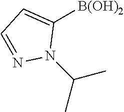 | 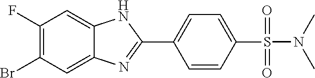 [SMILES: CN(C)S(=O)(=O)c1ccc(-c2nc3cc(Br)c(F)cc3[nH]2)cc1] | 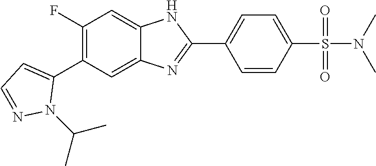 [SMILES: CC(C)n1nccc1-c1cc2nc(-c3ccc(S(=O)(=O)N(C)C)cc3)[nH]c2cc1F] | 4-(5-fluoro-6-(1-isopropyl-1H- pyrazol-5-yl)-1H-benzo[d] imidazol-2-yl)-N,N-dimethyl- benzenesulfonamide | 428.2 |
| 22 | 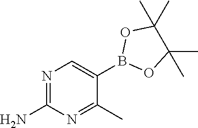 [SMILES: Cc1nc(N)ncc1B1OC(C)(C)C(C)(C)O1] | 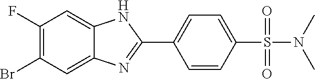 [SMILES: CN(C)S(=O)(=O)c1ccc(-c2nc3cc(Br)c(F)cc3[nH]2)cc1] | 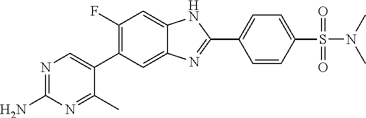 [SMILES: Cc1nc(N)ncc1-c1cc2nc(-c3ccc(S(=O)(=O)N(C)C)cc3)[nH]c2cc1F] | 4-(6-(2-amino-4-methylpyr- imid-5-yl)-5-fluoro-1H-benzo [d]imidazol-2-yl)-N,N-dimeth- yl-benzenesulfonamide | 427.1 |
| 23 | 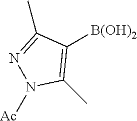 | 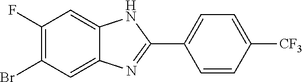 [SMILES: Fc1cc2[nH]c(-c3ccc(C(F)(F)F)cc3)nc2cc1Br] | 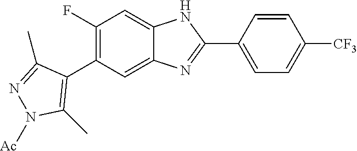 [SMILES: CC(=O)n1nc(C)c(-c2cc3nc(-c4ccc(C(F)(F)F)cc4)[nH]c3cc2F)c1C] | 1-(4-(6-fluoro-2-(4-(trifluoro- methyl)phenyl)-1H-benzo[d] imidazol-5-yl)-3,5-dimethyl- 1H-pyrazol-1-yl)ethan-1-one | 417.1 |
| 24 | 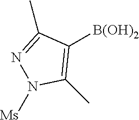 | 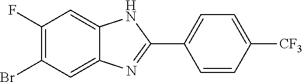 [SMILES: Fc1cc2[nH]c(-c3ccc(C(F)(F)F)cc3)nc2cc1Br] | 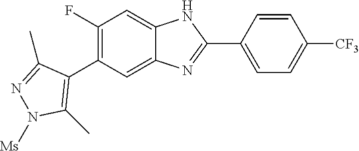 [SMILES: Cc1nn(S(C)(=O)=O)c(C)c1-c1cc2nc(-c3ccc(C(F)(F)F)cc3)[nH]c2cc1F] | 6-(3,5-dimethyl-1-(methyl- sulfonyl)-1H-pyrazo1-4-yl)-5- fluoro-2-(4-(trifluoromethyl)- phenyl)-1H-benzo[d]imidazole | 453.1 |
| 25 | 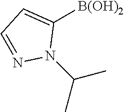 | 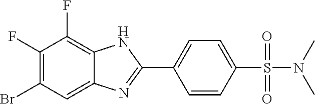 [SMILES: CN(C)S(=O)(=O)c1ccc(-c2nc3cc(Br)c(F)c(F)c3[nH]2)cc1] | 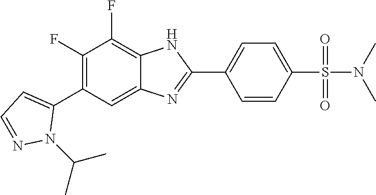 [SMILES: CC(C)n1nccc1-c1cc2nc(-c3ccc(S(=O)(=O)N(C)C)cc3)[nH]c2c(F)c1F] | 4-(4,5-difluoro-6-(1-isopropyl- 1H-pyrazol-5-yl)-1H-benzo[d] imidazol-2-yl)-N,N-dimethyl- benzenesulfonamide | 446.1 |
| 26 | 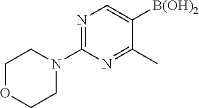 [SMILES: Cc1nc(N2CCOCC2)ncc1B(O)O] | 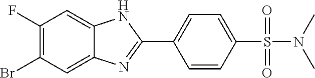 [SMILES: CN(C)S(=O)(=O)c1ccc(-c2nc3cc(Br)c(F)cc3[nH]2)cc1] | 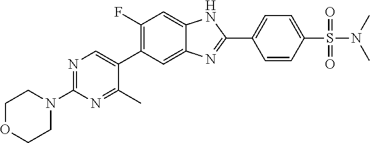 [SMILES: Cc1nc(N2CCOCC2)ncc1-c1cc2nc(-c3ccc(S(=O)(=O)N(C)C)cc3)[nH]c2cc1F] | 4-(5-fluoro-6-(4-methyl-2- morpholinopyrimid-5-yl)-1H- benzo[d]imidazol-2-yl)-N,N- dimethylbenzenesulfonamide | 497.2 |
| 27 | 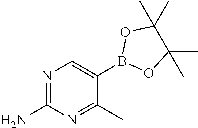 [SMILES: Cc1nc(N)ncc1B1OC(C)(C)C(C)(C)O1] | 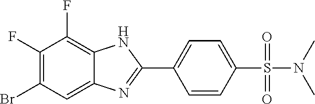 [SMILES: CN(C)S(=O)(=O)c1ccc(-c2nc3cc(Br)c(F)c(F)c3[nH]2)cc1] | 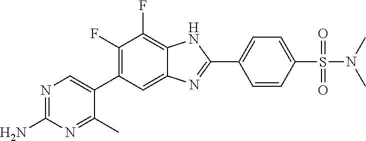 [SMILES: Cc1nc(N)ncc1-c1cc2nc(-c3ccc(S(=O)(=O)N(C)C)cc3)[nH]c2c(F)c1F] | 4-(6-(2-amino-4-methylpyr- imid-5-yl)-4,5-difluoro-1H- benzo[d]imidazol-2-yl)-N,N- dimethylbenzenesulfonamide | 445.1 |
| 28 | 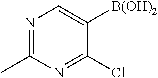 | 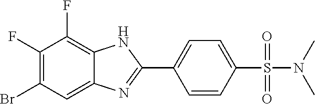 [SMILES: CN(C)S(=O)(=O)c1ccc(-c2nc3cc(Br)c(F)c(F)c3[nH]2)cc1] | 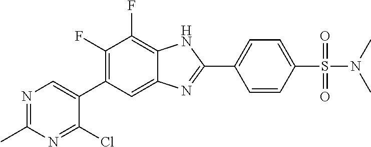 | 4-(6-(4-chloro-2-methylpyr- imid-5-yl)-4,5-difluoro-1H- benzo[d]imidazol-2-yl)-N,N- dimethylbenzenesulfonamide | 464.1 |
| 29 | 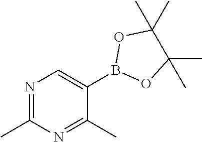 | 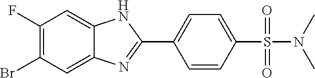 [SMILES: CN(C)S(=O)(=O)c1ccc(-c2nc3cc(Br)c(F)cc3[nH]2)cc1] | 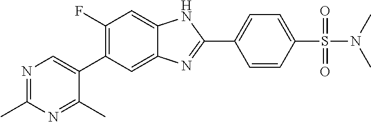 | 4-(6-(2,4-dimethylpyrimid-5- yl)-5-fluoro-1H-benzo[d] imidazol-2-yl)-N,N-dimethyl- benzenesulfonamide | 426.1 |
| 30 | 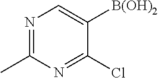 | 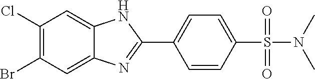 [SMILES: CN(C)S(=O)(=O)c1ccc(-c2nc3cc(Br)c(Cl)cc3[nH]2)cc1] | 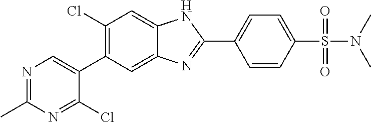 | 4-(5-chloro-6-(4-chloro-2- methylpyrimid-5-yl)-1H-benzo [d]imidazol-2-yl)-N,N-dimeth- ylbenzenesulfonamide | 462.1 |
| 31 | 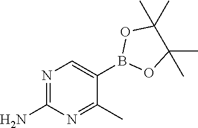 [SMILES: Cc1nc(N)ncc1B1OC(C)(C)C(C)(C)O1] | 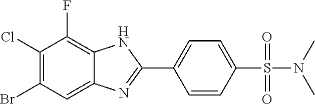 [SMILES: CN(C)S(=O)(=O)c1ccc(-c2nc3cc(Br)c(Cl)c(F)c3[nH]2)cc1] | 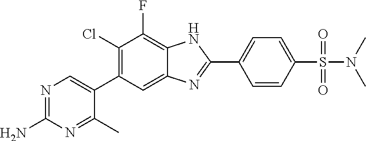 [SMILES: Cc1nc(N)ncc1-c1cc2nc(-c3ccc(S(=O)(=O)N(C)C)cc3)[nH]c2c(F)c1Cl] | 4-(6-(2-amino-4-methylpyr- imid-5-yl)-5-chloro-4-fluoro- 1H-benzo[d]imidazol-2-yl)- N,N-dimethylbenzenesulfon- amide | 461.1 |
| 32 | 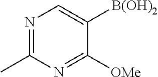 | 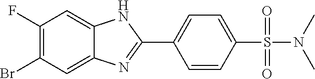 [SMILES: CN(C)S(=O)(=O)c1ccc(-c2nc3cc(Br)c(F)cc3[nH]2)cc1] | 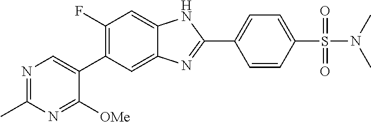 | 4-(5-fluoro-6-(4-methoxy-2- methylpyrimid-5-yl)-1H- benzo[d]imidazol-2-yl)-N,N- dimethylbenzenesulfonamide | 442.1 |
| 33 | 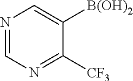 [SMILES: OB(O)c1cncnc1C(F)(F)F] | 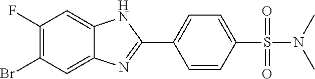 [SMILES: CN(C)S(=O)(=O)c1ccc(-c2nc3cc(Br)c(F)cc3[nH]2)cc1] | 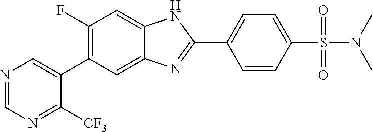 [SMILES: CN(C)S(=O)(=O)c1ccc(-c2nc3cc(-c4cncnc4C(F)(F)F)c(F)cc3[nH]2)cc1] | 4-(5-fluoro-6-(4-(trifluoro- methyl)pyrimid-5-yl)-1H-benzo [d]imidazol-2-yl)-N,N-dimeth- ylbenzenesulfonamide | 466.1 |
| 34 | 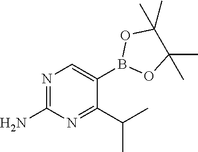 [SMILES: CC(C)c1nc(N)ncc1B1OC(C)(C)C(C)(C)O1] | 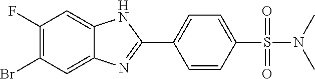 [SMILES: CN(C)S(=O)(=O)c1ccc(-c2nc3cc(Br)c(F)cc3[nH]2)cc1] | 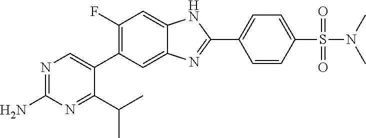 [SMILES: CC(C)c1nc(N)ncc1-c1cc2nc(-c3ccc(S(=O)(=O)N(C)C)cc3)[nH]c2cc1F] | 4-(6-(2-amino-4-isopropyl- pyrimid-5-yl)-5-fluoro-1H- benzo[d]imidazol-2-yl)-N,N- dimethylbenzenesulfonamide | 455.2 |
| 35 | 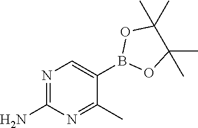 [SMILES: Cc1nc(N)ncc1B1OC(C)(C)C(C)(C)O1] | 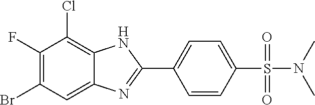 [SMILES: CN(C)S(=O)(=O)c1ccc(-c2nc3cc(Br)c(F)c(Cl)c3[nH]2)cc1] | 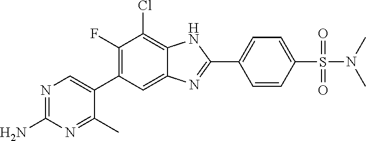 [SMILES: Cc1nc(N)ncc1-c1cc2nc(-c3ccc(S(=O)(=O)N(C)C)cc3)[nH]c2c(Cl)c1F] | 4-(6-(2-amino-4-methylpyr- imid-5-yl)-4-chloro-5-fluoro- 1H-benzo[d]imidazol-2-yl)- N,N-dimethylbenzenesulfon- amide | 461.1 |
| 36 | 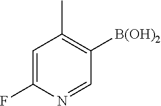 | 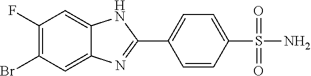 [SMILES: NS(=O)(=O)c1ccc(-c2nc3cc(Br)c(F)cc3[nH]2)cc1] | 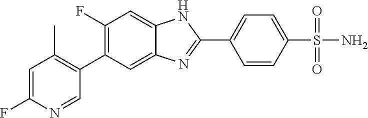 [SMILES: Cc1cc(F)ncc1-c1cc2nc(-c3ccc(S(N)(=O)=O)cc3)[nH]c2cc1F] | 4-(5-fluoro-6-(6-fluoro-4- methylpyridin-3-yl)-1H-benzo [d]imidazol-2-l)benzenesulfon- amide | 401.1 |
| 37 | 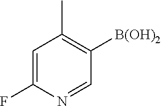 | 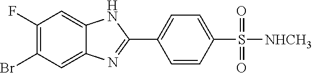 [SMILES: CNS(=O)(=O)c1ccc(-c2nc3cc(Br)c(F)cc3[nH]2)cc1] | 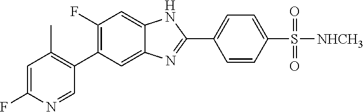 [SMILES: CNS(=O)(=O)c1ccc(-c2nc3cc(-c4cnc(F)cc4C)c(F)cc3[nH]2)cc1] | 4-(5-fluoro-6-(6-fluoro-4- methylpyridin-3-yl)-1H-benzo [d]imidazol-2-yl)-N-methyl- benzenesulfonamide | 415.1 |
| 38 | 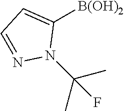 | 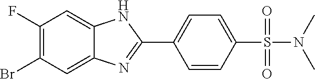 [SMILES: CN(C)S(=O)(=O)c1ccc(-c2nc3cc(Br)c(F)cc3[nH]2)cc1] | 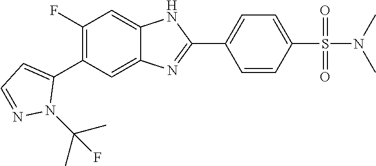 [SMILES: CN(C)S(=O)(=O)c1ccc(-c2nc3cc(-c4ccnn4C(C)(C)F)c(F)cc3[nH]2)cc1] | 4-(5-fluoro-6-(1-(2-fluoropro- pan-2-yl)-1H-pyrazol-5-yl)- 1H-benzo[d]imidazol-2-yl)- N,N-dimethylbenzenesulfon- amide | 446.1 |
| 39 | 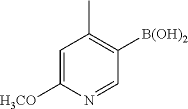 | 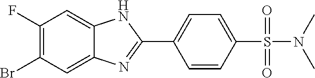 [SMILES: CN(C)S(=O)(=O)c1ccc(-c2nc3cc(Br)c(F)cc3[nH]2)cc1] | 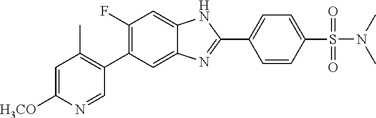 [SMILES: COc1cc(C)c(-c2cc3nc(-c4ccc(S(=O)(=O)N(C)C)cc4)[nH]c3cc2F)cn1] | 4-(5-fluoro-6-(6-methoxy-4- methylpyridin-3-yl)-1H-benzo [d]imidazol-2-yl)-N,N-dimeth- ylbenzenesulfonamide | 441.1 |
| 40 | 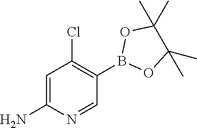 [SMILES: CC1(C)OB(c2cnc(N)cc2Cl)OC1(C)C] | 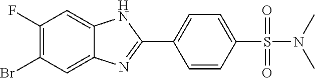 [SMILES: CN(C)S(=O)(=O)c1ccc(-c2nc3cc(Br)c(F)cc3[nH]2)cc1] | 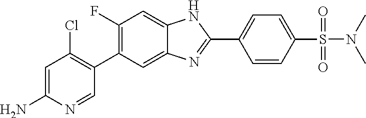 [SMILES: CN(C)S(=O)(=O)c1ccc(-c2nc3cc(-c4cnc(N)cc4Cl)c(F)cc3[nH]2)cc1] | 4-(6-(6-amino-4-chloropyr- idin-3-yl)-5-fluoro-1H-benzo [d]imidazol-2-yl)-N,N-dimeth- ylbenzenesuIfonamide | 446.1 |
| 41 | 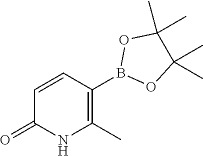 [SMILES: Cc1[nH]c(=O)ccc1B1OC(C)(C)C(C)(C)O1] | 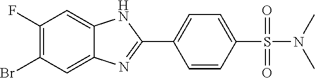 [SMILES: CN(C)S(=O)(=O)c1ccc(-c2nc3cc(Br)c(F)cc3[nH]2)cc1] | 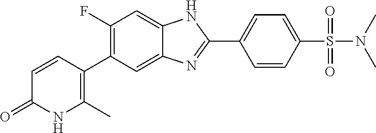 [SMILES: Cc1[nH]c(=O)ccc1-c1cc2nc(-c3ccc(S(=O)(=O)N(C)C)cc3)[nH]c2cc1F] | 4-(5-fluoro-6-(2-methyl-6- oxo-1,6-dihydropyridin-3-yl)- 1H-benzo[d]imidazol-2-yl)- N,N-dimethylbenzenesulfon- amide | 427.1 |
| 42 | 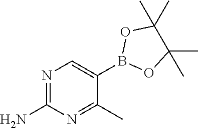 [SMILES: Cc1nc(N)ncc1B1OC(C)(C)C(C)(C)O1] | 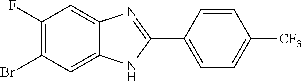 [SMILES: Fc1cc2nc(-c3ccc(C(F)(F)F)cc3)[nH]c2cc1Br] | 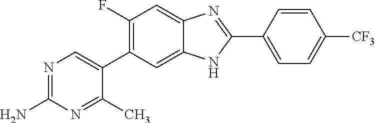 [SMILES: Cc1nc(N)ncc1-c1cc2[nH]c(-c3ccc(C(F)(F)F)cc3)nc2cc1F] | 5-(5-fluoro-2-(4-(trifluoro- methyl)phenyl)-1H-benzo[d] imidazol-6-yl)-4-methylpyrim- idine-2-amine | 388.1 |
| 43 | 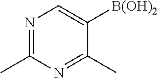 | 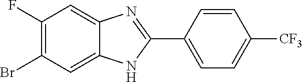 [SMILES: Fc1cc2nc(-c3ccc(C(F)(F)F)cc3)[nH]c2cc1Br] | 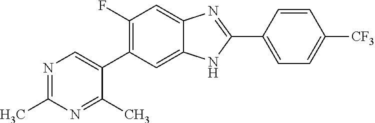 [SMILES: Cc1ncc(-c2cc3[nH]c(-c4ccc(C(F)(F)F)cc4)nc3cc2F)c(C)n1] | 6-(2,4-dimethylpyrimid-5-yl)- 5-fluoro-2-(4-(trifluorometh- yl)phenyl)-1H-benzo[d]imid- azole | 387.1 |
| 44 | 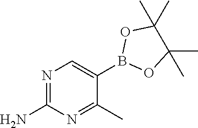 [SMILES: Cc1nc(N)ncc1B1OC(C)(C)C(C)(C)O1] | 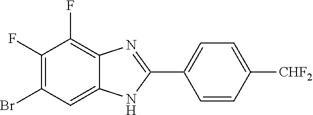 [SMILES: Fc1c(Br)cc2[nH]c(-c3ccc(C(F)F)cc3)nc2c1F] | 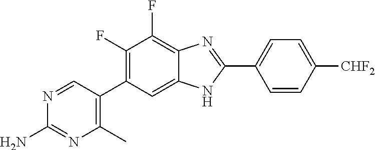 [SMILES: Cc1nc(N)ncc1-c1cc2[nH]c(-c3ccc(C(F)F)cc3)nc2c(F)c1F] | 5-(2-(4-(difluoromethyl)phen- yl)-4,5-difluoro-1H-benzo[d] imidazol-6-yl)-4-methylpyr- imidine-2-amine | 388.1 |
| 45 | 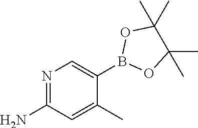 [SMILES: Cc1cc(N)ncc1B1OC(C)(C)C(C)(C)O1] | 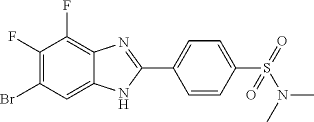 [SMILES: CN(C)S(=O)(=O)c1ccc(-c2nc3c(F)c(F)c(Br)cc3[nH]2)cc1] | 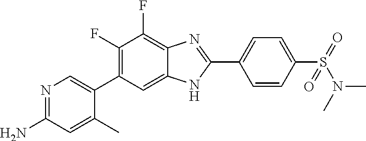 [SMILES: Cc1cc(N)ncc1-c1cc2[nH]c(-c3ccc(S(=O)(=O)N(C)C)cc3)nc2c(F)c1F] | 4-(6-(6-amino-4-methylpyr- idin-3-yl)-4,5-difluoro-1H- benzo[d]imidazol-2-yl)-N,N- dimethylbenzenesulfonamide | 444.1 |
| 46 | 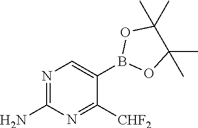 [SMILES: CC1(C)OB(c2cnc(N)nc2C(F)F)OC1(C)C] | 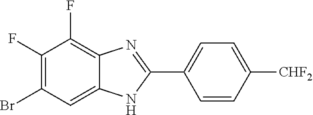 [SMILES: Fc1c(Br)cc2[nH]c(-c3ccc(C(F)F)cc3)nc2c1F] | 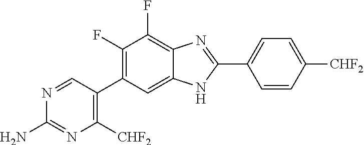 [SMILES: Nc1ncc(-c2cc3[nH]c(-c4ccc(C(F)F)cc4)nc3c(F)c2F)c(C(F)F)n1] | 4-(difluoromethyl)-5-(2-(4- (difluoromethyl)phenyl)-4,5- difluoro-1H-benzo[d]imid- azol-6-yl)pyrimidine-2-amine | 424.1 |
| 47 | 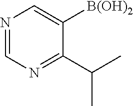 | 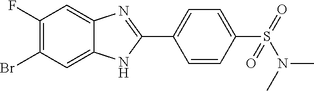 [SMILES: CN(C)S(=O)(=O)c1ccc(-c2nc3cc(F)c(Br)cc3[nH]2)cc1] | 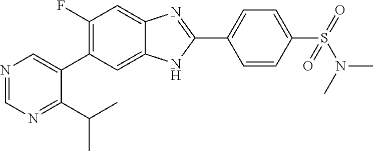 [SMILES: CC(C)c1ncncc1-c1cc2[nH]c(-c3ccc(S(=O)(=O)N(C)C)cc3)nc2cc1F] | 4-(5-fluoro-6-(4-isopropyl- pyrimid-5-yl)-1H-benzo[d] imidazol-2-yl)-N,N-dimethyl- benzenesulfonamide | 440.1 |
| 48 | 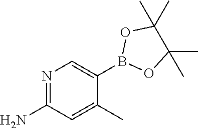 [SMILES: Cc1cc(N)ncc1B1OC(C)(C)C(C)(C)O1] | 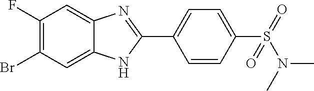 [SMILES: CN(C)S(=O)(=O)c1ccc(-c2nc3cc(F)c(Br)cc3[nH]2)cc1] | 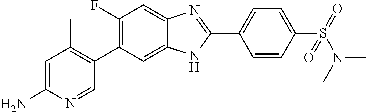 [SMILES: Cc1cc(N)ncc1-c1cc2[nH]c(-c3ccc(S(=O)(=O)N(C)C)cc3)nc2cc1F] | 4-(6-(6-amino-4-methylpyri- din-3-yl)-5-fluoro-1H-benzo [d]imidazol-2-yl)-N,N-dimeth- ylbenzenesulfonamide | 426.2 |
| 49 | 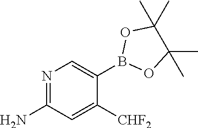 [SMILES: CC1(C)OB(c2cnc(N)cc2C(F)F)OC1(C)C] | 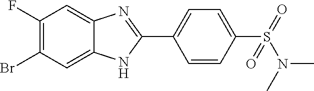 [SMILES: CN(C)S(=O)(=O)c1ccc(-c2nc3cc(F)c(Br)cc3[nH]2)cc1] | 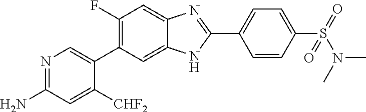 [SMILES: CN(C)S(=O)(=O)c1ccc(-c2nc3cc(F)c(-c4cnc(N)cc4C(F)F)cc3[nH]2)cc1] | 4-(6-(6-amino-4-(difluoro- methyl)pyridin-3-yl)-5-fluoro- 1H-benzo[d]imidazol-2-yl)- N,N-dimethylbenzenesulfon- amide | 462.1 |
| 50 | 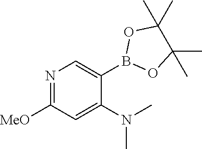 [SMILES: COc1cc(N(C)C)c(B2OC(C)(C)C(C)(C)O2)cn1] | 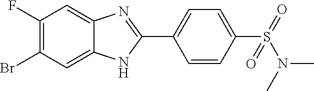 [SMILES: CN(C)S(=O)(=O)c1ccc(-c2nc3cc(F)c(Br)cc3[nH]2)cc1] | 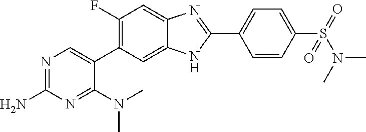 [SMILES: CN(C)c1nc(N)ncc1-c1cc2[nH]c(-c3ccc(S(=O)(=O)N(C)C)cc3)nc2cc1F] | 4-(6-(4-(dimethylamino)-2- methoxypyrimid-5-yl)-5- fluoro-1H-benzo[d]imidazol- 2-yl)-N,N-dimethylbenzene- sulfonamide | 471.2 |
| 51 | 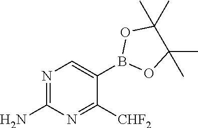 [SMILES: CC1(C)OB(c2cnc(N)nc2C(F)F)OC1(C)C] | 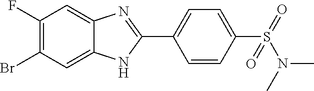 [SMILES: CN(C)S(=O)(=O)c1ccc(-c2nc3cc(F)c(Br)cc3[nH]2)cc1] | 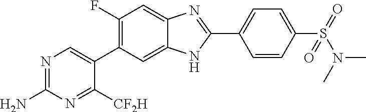 [SMILES: CN(C)S(=O)(=O)c1ccc(-c2nc3cc(F)c(-c4cnc(N)nc4C(F)F)cc3[nH]2)cc1] | 4-(6-(4-(difluoromethyl)-2- aminopyrimid-5-yl)-5-fluoro- 1H-benzo[d]imidazol-2-yl)- N,N-dimethylbenzenesulfon- amide | 463.2 |
| 52 | 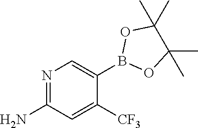 [SMILES: CC1(C)OB(c2cnc(N)cc2C(F)(F)F)OC1(C)C] | 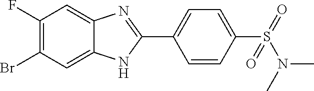 [SMILES: CN(C)S(=O)(=O)c1ccc(-c2nc3cc(F)c(Br)cc3[nH]2)cc1] | 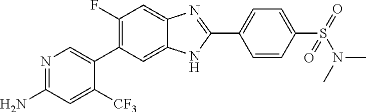 [SMILES: CN(C)S(=O)(=O)c1ccc(-c2nc3cc(F)c(-c4cnc(N)cc4C(F)(F)F)cc3[nH]2)cc1] | 4-(6-(4-(trifluoromethyl)-6- aminopyridin-3-yl)-5-fluoro- 1H-benzo[d]imidazol-2-yl)- N,N-dimethylbenzenesulfon- amide | 480.1 |
| 53 | 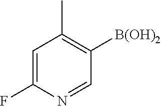 | 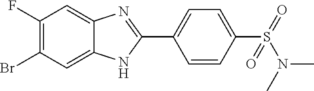 [SMILES: CN(C)S(=O)(=O)c1ccc(-c2nc3cc(F)c(Br)cc3[nH]2)cc1] | 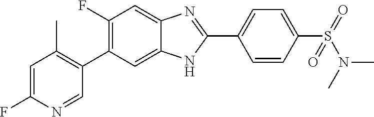 [SMILES: Cc1cc(F)ncc1-c1cc2[nH]c(-c3ccc(S(=O)(=O)N(C)C)cc3)nc2cc1F] | 4-(6-(4-methyl-6-fluoropyr- idin-3-yl)-5-fluoro-1H-benzo [d]imidazol-2-yl)-N,N-dimeth- ylbenzenesulfonamide | 429.1 |
| 54 | 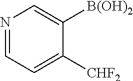 [SMILES: OB(O)c1cnccc1C(F)F] | 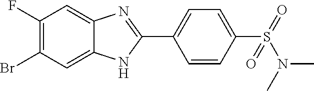 [SMILES: CN(C)S(=O)(=O)c1ccc(-c2nc3cc(F)c(Br)cc3[nH]2)cc1] | 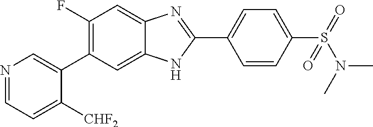 [SMILES: CN(C)S(=O)(=O)c1ccc(-c2nc3cc(F)c(-c4cnccc4C(F)F)cc3[nH]2)cc1] | 4-(6-(4-(difluoromethyl)pyr- idin-3-yl)-5-fluoro-1H-benzo [d]imidazol-2-yl)-N,N- dimethylbenzenesulfonamide (compound 54) | 47.1 |
| 55 | 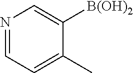 | 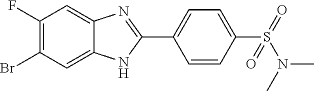 [SMILES: CN(C)S(=O)(=O)c1ccc(-c2nc3cc(F)c(Br)cc3[nH]2)cc1] | 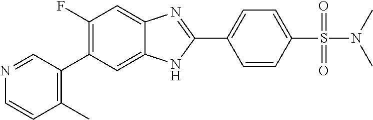 [SMILES: Cc1ccncc1-c1cc2[nH]c(-c3ccc(S(=O)(=O)N(C)C)cc3)nc2cc1F] | 4-(6-(4-methylpyridin-3-yl)-5- fluoro-1H-benzo[d]imidazol- 2-yl)-N,N-dimethylbenzene- sulfonamide | 411.2 |
| 56 | 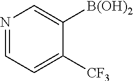 [SMILES: OB(O)c1cnccc1C(F)(F)F] | 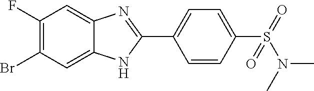 [SMILES: CN(C)S(=O)(=O)c1ccc(-c2nc3cc(F)c(Br)cc3[nH]2)cc1] | 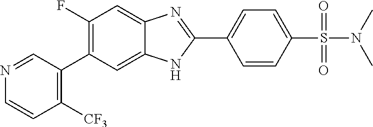 [SMILES: CN(C)S(=O)(=O)c1ccc(-c2nc3cc(F)c(-c4cnccc4C(F)(F)F)cc3[nH]2)cc1] | 4-(6-(4-(trifluoromethyl)pyr- idin-3-yl)-5-fluoro-1H-benzo [d]imidazol-2-yl)-N,N-dimeth- ylbenzenesulfonamide | 465.1 |
| 57 | 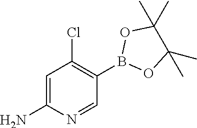 [SMILES: CC1(C)OB(c2cnc(N)cc2Cl)OC1(C)C] | 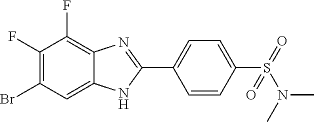 [SMILES: CN(C)S(=O)(=O)c1ccc(-c2nc3c(F)c(F)c(Br)cc3[nH]2)cc1] | 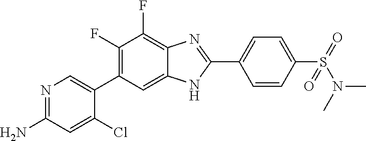 [SMILES: CN(C)S(=O)(=O)c1ccc(-c2nc3c(F)c(F)c(-c4cnc(N)cc4Cl)cc3[nH]2)cc1] | 4-(6-(6-amino-4-chloropyri- din-3-yl)-4,5-difluoro-1H- benzo[d]imidazol-2-yl)-N,N- dimethylbenzenesulfonamide | 464.1 |
| 58 | 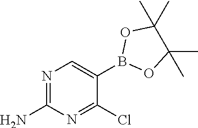 [SMILES: CC1(C)OB(c2cnc(N)nc2Cl)OC1(C)C] | 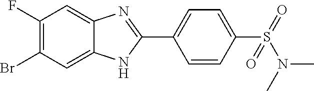 [SMILES: CN(C)S(=O)(=O)c1ccc(-c2nc3cc(F)c(Br)cc3[nH]2)cc1] | 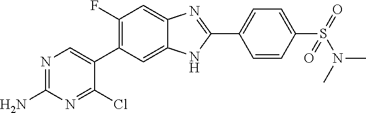 [SMILES: CN(C)S(=O)(=O)c1ccc(-c2nc3cc(F)c(-c4cnc(N)nc4Cl)cc3[nH]2)cc1] | 4-(6-(2-amino-4-chloropyr- imid-5-yl)-5-fluoro-1H-benzo [d]imidazol-2-yl)-N,N-dimeth- ylbenzenesulfonamide | 447.1 |
| 59 | 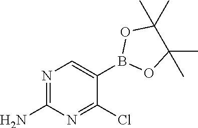 [SMILES: CC1(C)OB(c2cnc(N)nc2Cl)OC1(C)C] | 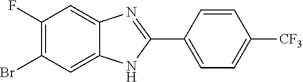 [SMILES: Fc1cc2nc(-c3ccc(C(F)(F)F)cc3)[nH]c2cc1Br] | 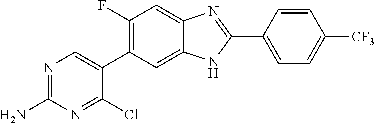 [SMILES: Nc1ncc(-c2cc3[nH]c(-c4ccc(C(F)(F)F)cc4)nc3cc2F)c(Cl)n1] | 5-(5-fluoro-2-(4-(trifluoro- methyl)phenyl)-1H-benzo[d] imidazol-6-yl)-4-chloropyr- imidine-2-amine | 408.1 |
| 60 | 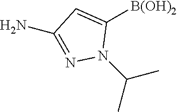 | 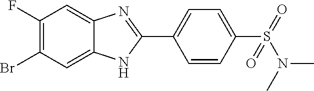 [SMILES: CN(C)S(=O)(=O)c1ccc(-c2nc3cc(F)c(Br)cc3[nH]2)cc1] | 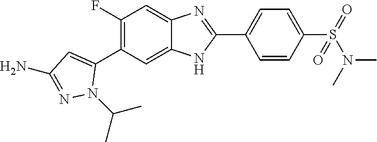 [SMILES: CC(C)n1nc(N)cc1-c1cc2[nH]c(-c3ccc(S(=O)(=O)N(C)C)cc3)nc2cc1F] | 4-(6-(1-isopropyl-3-amino- 1H-pyrazol-5-yl)-5-fluoro-1H- benzo[d]imidazol-2-yl)-N,N- dimethylbenzenesulfonamide | 443.1 |
| 61 | 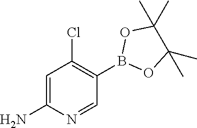 [SMILES: CC1(C)OB(c2cnc(N)cc2Cl)OC1(C)C] | 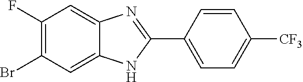 [SMILES: Fc1cc2nc(-c3ccc(C(F)(F)F)cc3)[nH]c2cc1Br] | 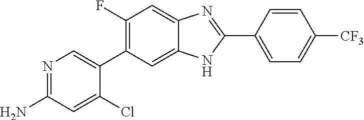 [SMILES: Nc1cc(Cl)c(-c2cc3[nH]c(-c4ccc(C(F)(F)F)cc4)nc3cc2F)cn1] | 2-(4-(trifluoromethyl)phenyl)- 6-(6-amino-4-chloropyridin- 3-yl)-5-fluoro-1H-benzo[d] imidazole | 407.1 |
| 62 | 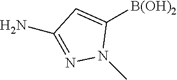 | 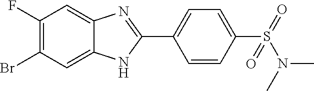 [SMILES: CN(C)S(=O)(=O)c1ccc(-c2nc3cc(F)c(Br)cc3[nH]2)cc1] | 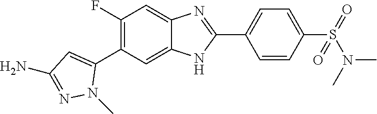 [SMILES: CN(C)S(=O)(=O)c1ccc(-c2nc3cc(F)c(-c4cc(N)nn4C)cc3[nH]2)cc1] | 4-(6-(1-methyl-3-amino-1H- pyrazol-5-yl)-5-fluoro-1H- benzo[d]imidazol-2-yl)-N,N- dimethylbenzenesulfonamide | 415.2 |
| 63 | 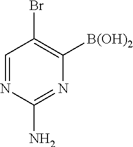 [SMILES: Nc1ncc(Br)c(B(O)O)n1] | 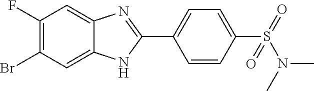 [SMILES: CN(C)S(=O)(=O)c1ccc(-c2nc3cc(F)c(Br)cc3[nH]2)cc1] | 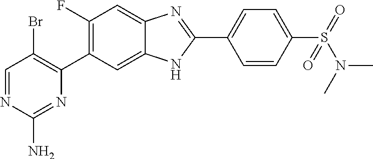 [SMILES: CN(C)S(=O)(=O)c1ccc(-c2nc3cc(F)c(-c4nc(N)ncc4Br)cc3[nH]2)cc1] | 4-(6-(2-amino-4-bromopyr- imid-3-yl)-5-fluoro-1H-benzo [d]imidazol-2-yl)-N,N-dimeth- ylbenzenesulfonamide | 491.2 |
| 64 | 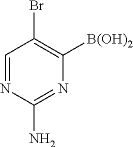 [SMILES: Nc1ncc(Br)c(B(O)O)n1] | 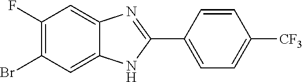 [SMILES: Fc1cc2nc(-c3ccc(C(F)(F)F)cc3)[nH]c2cc1Br] | 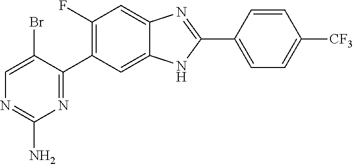 [SMILES: Nc1ncc(Br)c(-c2cc3[nH]c(-c4ccc(C(F)(F)F)cc4)nc3cc2F)n1] | 3-(2-(4-(trifluoromethyl)phen- yl)-5-fluoro-1H-benzo[d]imid- azol-6-yl)-4-bromopyrimidine- 2-amine | 452.1 |
| 65 | 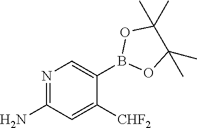 [SMILES: CC1(C)OB(c2cnc(N)cc2C(F)F)OC1(C)C] | 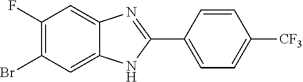 [SMILES: Fc1cc2nc(-c3ccc(C(F)(F)F)cc3)[nH]c2cc1Br] | 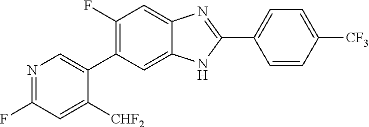 [SMILES: Fc1cc(C(F)F)c(-c2cc3[nH]c(-c4ccc(C(F)(F)F)cc4)nc3cc2F)cn1] | 2-(4-(trifluoromethyl)phen- yl)-6-(4-(difluoromethyl)-6- fluoropyridin-3-yl)-5-fluoro- 1H-benzo[d]imidazole | 426.1 |
| 66 | 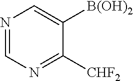 [SMILES: OB(O)c1cncnc1C(F)F] | 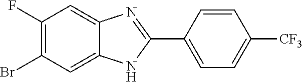 [SMILES: Fc1cc2nc(-c3ccc(C(F)(F)F)cc3)[nH]c2cc1Br] | 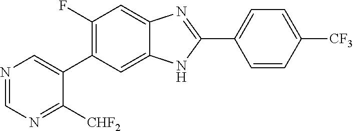 [SMILES: Fc1cc2nc(-c3ccc(C(F)(F)F)cc3)[nH]c2cc1-c1cncnc1C(F)F] | 5-(2-(4-(trifluoromethyl)phen- yl)-5-fluoro-1H-benzo[d]imid- azol-6-yl)-4-(difluoromethyl)- pyrimidine | 409.2 |
| 67 | 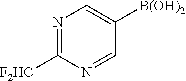 [SMILES: OB(O)c1cnc(C(F)F)nc1] | 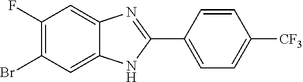 [SMILES: Fc1cc2nc(-c3ccc(C(F)(F)F)cc3)[nH]c2cc1Br] | 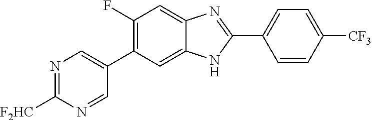 [SMILES: Fc1cc2nc(-c3ccc(C(F)(F)F)cc3)[nH]c2cc1-c1cnc(C(F)F)nc1] | 5-(2-(4-(trifluoromethyl)phen- yl)-5-fluoro-1H-benzo[d]imid- azol-6-yl)-2-(difluoromethyl)- pyrimidine | 409.2 |
| 68 | 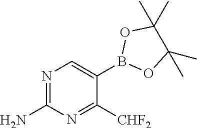 [SMILES: CC1(C)OB(c2cnc(N)nc2C(F)F)OC1(C)C] | 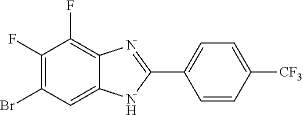 [SMILES: Fc1c(Br)cc2[nH]c(-c3ccc(C(F)(F)F)cc3)nc2c1F] | 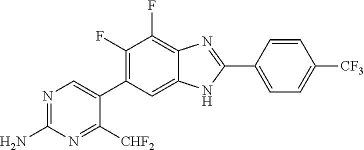 [SMILES: Nc1ncc(-c2cc3[nH]c(-c4ccc(C(F)(F)F)cc4)nc3c(F)c2F)c(C(F)F)n1] | 5-(2-(4-(trifluoromethyl)phen- yl)-4,5-difluoro-1H-benzo[d]- imidazol-6-yl)-4-(difluoro- methyl)pyrimidine-2-amine | 442.1 |
| 69 | 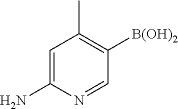 | 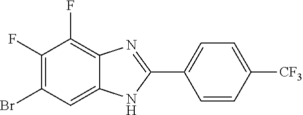 [SMILES: Fc1c(Br)cc2[nH]c(-c3ccc(C(F)(F)F)cc3)nc2c1F] | 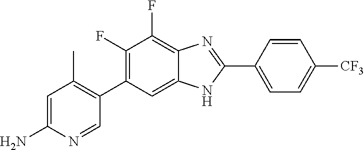 [SMILES: Cc1cc(N)ncc1-c1cc2[nH]c(-c3ccc(C(F)(F)F)cc3)nc2c(F)c1F] | 2-(4-(trifluoromethyl)phenyl)- 6-(4-methyl-6-aminopyridin- 3-yl)-4,5-difluoro-1H-benzo [d]imidazole | 405.2 |
| 70 | 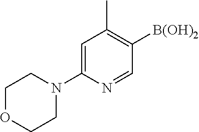 [SMILES: Cc1cc(N2CCOCC2)ncc1B(O)O] | 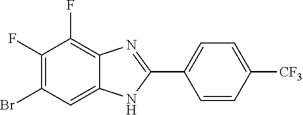 [SMILES: Fc1c(Br)cc2[nH]c(-c3ccc(C(F)(F)F)cc3)nc2c1F] | 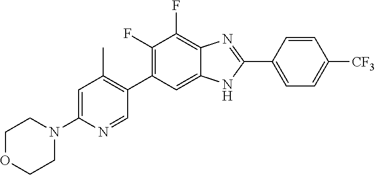 [SMILES: Cc1cc(N2CCOCC2)ncc1-c1cc2[nH]c(-c3ccc(C(F)(F)F)cc3)nc2c(F)c1F] | 4-(4-methyl-5-(2-(4-(trifluoro- methyl)phenyl)-4,5-difluoro- 1H-benzo[d]imidazol-6-yl)- pyrid-2-yl)morpholine | 475.2 |
| 71 | 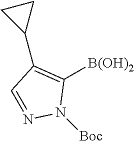 [SMILES: CC(C)(C)OC(=O)n1ncc(C2CC2)c1B(O)O] | 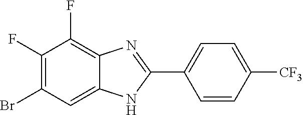 [SMILES: Fc1c(Br)cc2[nH]c(-c3ccc(C(F)(F)F)cc3)nc2c1F] | 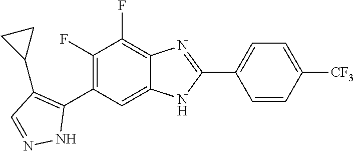 [SMILES: Fc1c(-c2[nH]ncc2C2CC2)cc2[nH]c(-c3ccc(C(F)(F)F)cc3)nc2c1F] | 2-(4-(trifluoromethyl)phenyl)- 6-(4-cyclopropyl-1H-pyrazol- 5-yl)-4,5-difluoro-1H-benzo [d]imidazole | 405.2 |
| 72 | 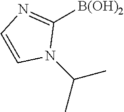 | 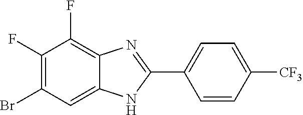 [SMILES: Fc1c(Br)cc2[nH]c(-c3ccc(C(F)(F)F)cc3)nc2c1F] | 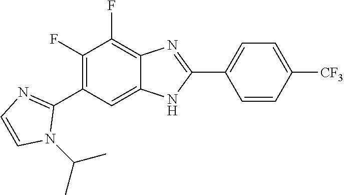 [SMILES: CC(C)n1ccnc1-c1cc2[nH]c(-c3ccc(C(F)(F)F)cc3)nc2c(F)c1F] | 2-(4-(trifluoromethyl)phenyl)- 6-(1-isopropyl-1H-imidazol- 2-yl)-4,5-difluoro-1H-benzo[d] imidazole | 407.1 |
| 73 | 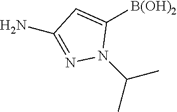 | 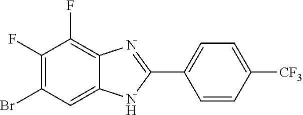 [SMILES: Fc1c(Br)cc2[nH]c(-c3ccc(C(F)(F)F)cc3)nc2c1F] | 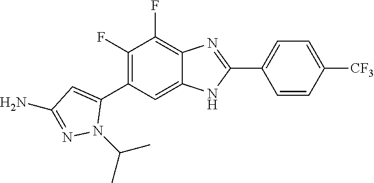 [SMILES: CC(C)n1nc(N)cc1-c1cc2[nH]c(-c3ccc(C(F)(F)F)cc3)nc2c(F)c1F] | 2-(4-(trifluoromethyl)phenyl)- 6-(1-isopropyl-3-amino-1H- pyrazol-5-yl)-4,5-difluoro-1H- benzo[d]imidazole | 422.2 |
| 74 | 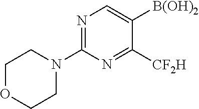 [SMILES: OB(O)c1cnc(N2CCOCC2)nc1C(F)F] | 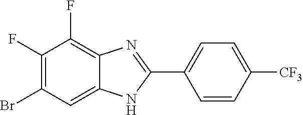 [SMILES: Fc1c(Br)cc2[nH]c(-c3ccc(C(F)(F)F)cc3)nc2c1F] | 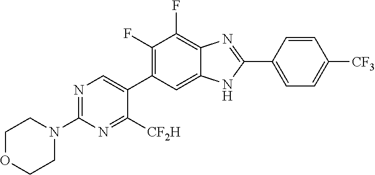 [SMILES: Fc1c(-c2cnc(N3CCOCC3)nc2C(F)F)cc2[nH]c(-c3ccc(C(F)(F)F)cc3)nc2c1F] | 4-(5-(2-(4-(trifluoromethyl)- phenyl)-4,5-difluoro-1H-benzo [d]imidazol-6-yl)-4-(difluoro- methyl)pyrid-2-yl)morpholine | 512.2 |
| 75 | 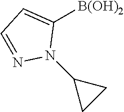 [SMILES: OB(O)c1ccnn1C1CC1] | 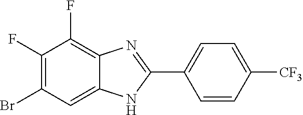 [SMILES: Fc1c(Br)cc2[nH]c(-c3ccc(C(F)(F)F)cc3)nc2c1F] | 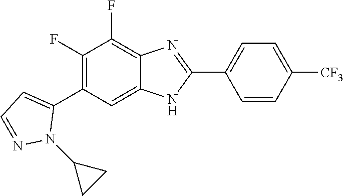 [SMILES: Fc1c(-c2ccnn2C2CC2)cc2[nH]c(-c3ccc(C(F)(F)F)cc3)nc2c1F] | 2-(4-(trifluoromethyl)phenyl)- 6-(1-cyclopropyl-1H-pyrazol- 5-yl)-4,5-difluoro-1H-benzo [d]imidazole | 405.1 |
| 76 | 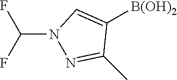 | 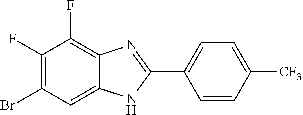 [SMILES: Fc1c(Br)cc2[nH]c(-c3ccc(C(F)(F)F)cc3)nc2c1F] | 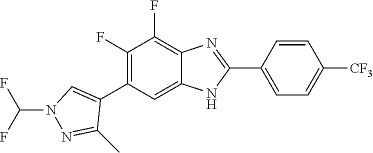 [SMILES: Cc1nn(C(F)F)cc1-c1cc2[nH]c(-c3ccc(C(F)(F)F)cc3)nc2c(F)c1F] | 2-(4-(trifluoromethyl)phenyl)- 6-(3-methyl-1-(difluorometh- yl)-1H-pyrazol-4-yl)-4,5- difluoro-1H-benzo[d]imidazole | 429.1 |
| 77 | 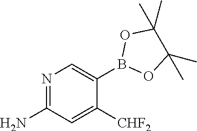 [SMILES: CC1(C)OB(c2cnc(N)cc2C(F)F)OC1(C)C] | 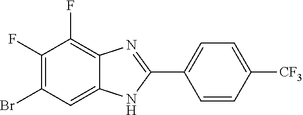 [SMILES: Fc1c(Br)cc2[nH]c(-c3ccc(C(F)(F)F)cc3)nc2c1F] | 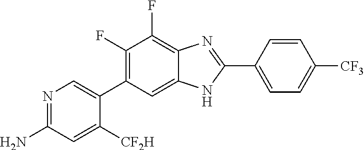 [SMILES: Nc1cc(C(F)F)c(-c2cc3[nH]c(-c4ccc(C(F)(F)F)cc4)nc3c(F)c2F)cn1] | 2-(4-(trifluoromethyl)phenyl)- 6-(4-(difluoromethyl)-6-amino- pyridin-3-yl)-4,5-difluoro-1H- benzo[d]imidazole | 441.1 |
| 78 | 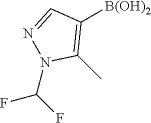 | 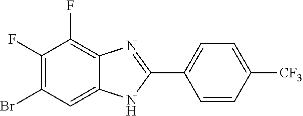 [SMILES: Fc1c(Br)cc2[nH]c(-c3ccc(C(F)(F)F)cc3)nc2c1F] | 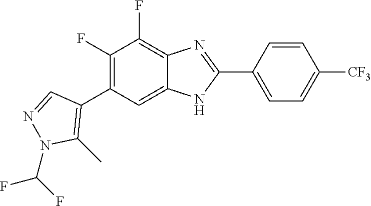 [SMILES: Cc1c(-c2cc3[nH]c(-c4ccc(C(F)(F)F)cc4)nc3c(F)c2F)cnn1C(F)F] | 2-(4-(trifluoromethyl)phenyl)- 6-(5-methyl-1-(difluorometh- yl)-1H-pyrazol-4-yl)-4,5- difluoro-1H-benzo[d]imidazole | 429.1 |
| 79 | 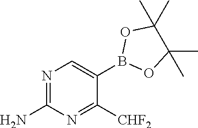 [SMILES: CC1(C)OB(c2cnc(N)nc2C(F)F)OC1(C)C] | 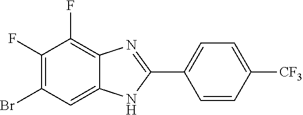 [SMILES: Fc1c(Br)cc2[nH]c(-c3ccc(C(F)(F)F)cc3)nc2c1F] | 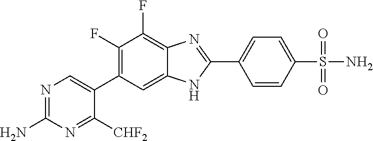 [SMILES: Nc1ncc(-c2cc3[nH]c(-c4ccc(S(N)(=O)=O)cc4)nc3c(F)c2F)c(C(F)F)n1] | 4-(6-(4-(difluoromethyl)-2- aminopyrimid-5-yl)-4,5- difluoro-1H-benzo[d]imid- azol-2-yl)-benzenesulfonamide | 453.1 |
| 80 | 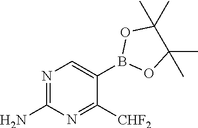 [SMILES: CC1(C)OB(c2cnc(N)nc2C(F)F)OC1(C)C] | 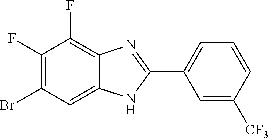 [SMILES: Fc1c(Br)cc2[nH]c(-c3cccc(C(F)(F)F)c3)nc2c1F] | 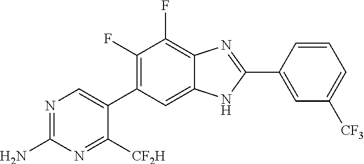 [SMILES: Nc1ncc(-c2cc3[nH]c(-c4cccc(C(F)(F)F)c4)nc3c(F)c2F)c(C(F)F)n1] | 5-(2-(3-(trifluoromethyl)phen- yl)-4,5-difluoro-1H-benzo[d] imidazol-6-yl)-4-(difluoro- methyl)pyrimidine-2-amine | 442.0 |
| 81 | 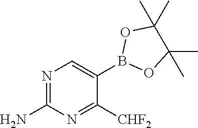 [SMILES: CC1(C)OB(c2cnc(N)nc2C(F)F)OC1(C)C] | 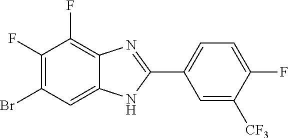 [SMILES: Fc1ccc(-c2nc3c(F)c(F)c(Br)cc3[nH]2)cc1C(F)(F)F] | 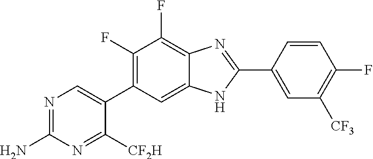 [SMILES: Nc1ncc(-c2cc3[nH]c(-c4ccc(F)c(C(F)(F)F)c4)nc3c(F)c2F)c(C(F)F)n1] | 5-(2-(3-(trifluoromethyl)-4- fluorophenyl)-4,5-difluoro- 1H-benzo[d]imidazol-6-yl)-4- (difluoromethyl)pyrimidine- 2-amine | 459.9 |
| 82 | 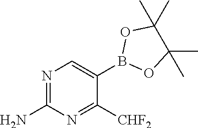 [SMILES: CC1(C)OB(c2cnc(N)nc2C(F)F)OC1(C)C] | 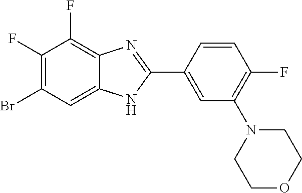 [SMILES: Fc1ccc(-c2nc3c(F)c(F)c(Br)cc3[nH]2)cc1N1CCOCC1] | 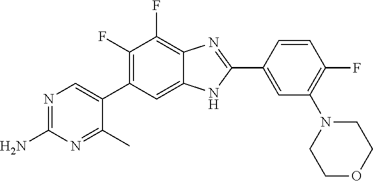 [SMILES: Cc1nc(N)ncc1-c1cc2[nH]c(-c3ccc(F)c(N4CCOCC4)c3)nc2c(F)c1F] | 4-methyl-5-(2-(3-morpholino- 4-fluorophenyl)-4,5-difluoro- 1H-benzo[d]imidazol-6-yl)- pyrimidine-2-amine | 441.2 |
| 83 | 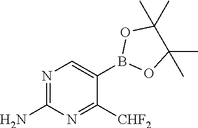 [SMILES: CC1(C)OB(c2cnc(N)nc2C(F)F)OC1(C)C] | 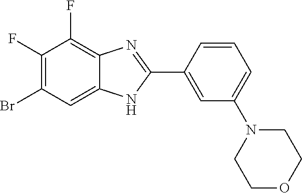 [SMILES: Fc1c(Br)cc2[nH]c(-c3cccc(N4CCOCC4)c3)nc2c1F] | 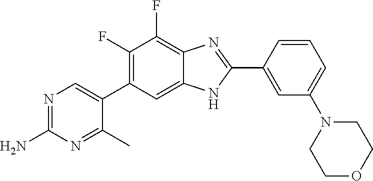 [SMILES: Cc1nc(N)ncc1-c1cc2[nH]c(-c3cccc(N4CCOCC4)c3)nc2c(F)c1F] | 4-methyl-5-(2-(3-morpholino- phenyl)-4,5-difluoro-1H-benzo [d]imidazol-6-yl)pyrimidine- 2-amine | 423.1 |
| 84 | 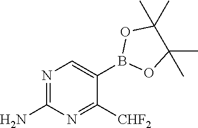 [SMILES: CC1(C)OB(c2cnc(N)nc2C(F)F)OC1(C)C] | 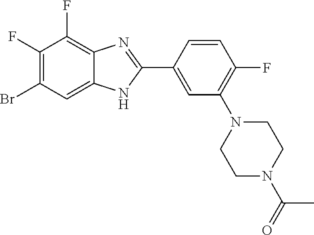 [SMILES: CC(=O)N1CCN(c2cc(-c3nc4c(F)c(F)c(Br)cc4[nH]3)ccc2F)CC1] | 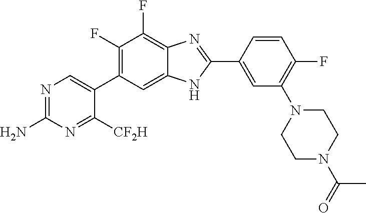 [SMILES: CC(=O)N1CCN(c2cc(-c3nc4c(F)c(F)c(-c5cnc(N)nc5C(F)F)cc4[nH]3)ccc2F)CC1] | 1-(4-(5-(6-(4-(difluorometh- yl)-2-aminopyrimid-5-yl)-4,5- difluoro-1H-benzo[d]imidazol- 2-yl)-2-fluorophenyl)-piper- azine-1-yl)-ethanone | 518.3 |
| 85 | 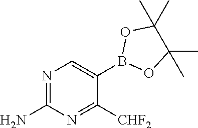 [SMILES: CC1(C)OB(c2cnc(N)nc2C(F)F)OC1(C)C] | 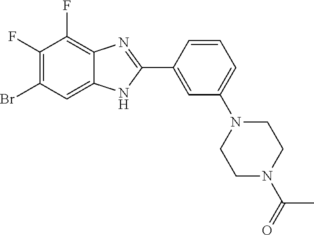 [SMILES: CC(=O)N1CCN(c2cccc(-c3nc4c(F)c(F)c(Br)cc4[nH]3)c2)CC1] | 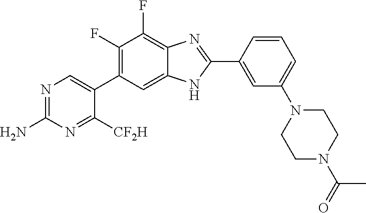 [SMILES: CC(=O)N1CCN(c2cccc(-c3nc4c(F)c(F)c(-c5cnc(N)nc5C(F)F)cc4[nH]3)c2)CC1] | 1-(4-(3-(6-(4-(difluorometh- yl)-2-aminopyrimid-5-yl)-4,5- difluoro-1H-benzo[d]imidazol- 2-yl)phenyl)-piperazine-1-yl)- ethan-1-one | 500.3 |
| 86 | 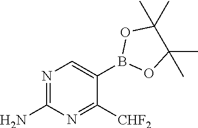 [SMILES: CC1(C)OB(c2cnc(N)nc2C(F)F)OC1(C)C] | 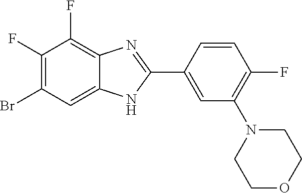 [SMILES: Fc1ccc(-c2nc3c(F)c(F)c(Br)cc3[nH]2)cc1N1CCOCC1] | 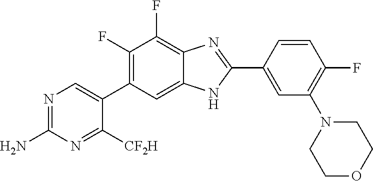 [SMILES: Nc1ncc(-c2cc3[nH]c(-c4ccc(F)c(N5CCOCC5)c4)nc3c(F)c2F)c(C(F)F)n1] | 5-(2-(3-morpholino-4-fluoro- phenyl)-4,5-difluoro-1H-benzo [d]imidazol-6-yl)-4-(difluoro- methyl)pyrimidine-2-amine | 477.3 |
| 87 | 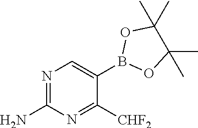 [SMILES: CC1(C)OB(c2cnc(N)nc2C(F)F)OC1(C)C] | 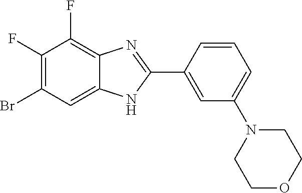 [SMILES: Fc1c(Br)cc2[nH]c(-c3cccc(N4CCOCC4)c3)nc2c1F] | 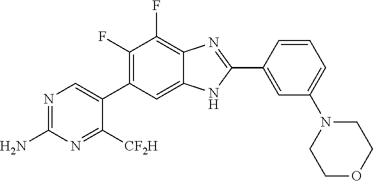 [SMILES: Nc1ncc(-c2cc3[nH]c(-c4cccc(N5CCOCC5)c4)nc3c(F)c2F)c(C(F)F)n1] | 5-(2-(3-morpholinophenyl)- 4,5-difluoro-1H-benzo[d]imid- azol-6-yl)-4-(difluoromethyl) pyrimidine-2-amine | 459.3 |
| 88 | 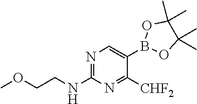 | 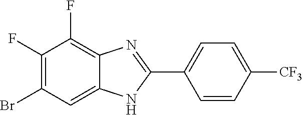 [SMILES: Fc1c(Br)cc2[nH]c(-c3ccc(C(F)(F)F)cc3)nc2c1F] | 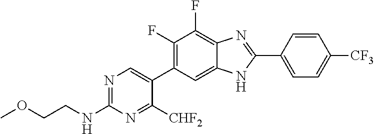 [SMILES: COCCNc1ncc(-c2cc3[nH]c(-c4ccc(C(F)(F)F)cc4)nc3c(F)c2F)c(C(F)F)n1] | 5-(2-(4-(trifluoromethyl)phen- yl)-4,5-difluoro-1H-benzo[d] imidazol-6-yl)-4-(difluoro- methyl)-N-(2-methoxyethyl)- pyrimidine-2-amine | 500.4 |
| 89 | 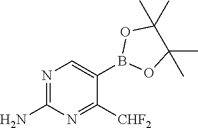 [SMILES: CC1(C)OB(c2cnc(N)nc2C(F)F)OC1(C)C] | 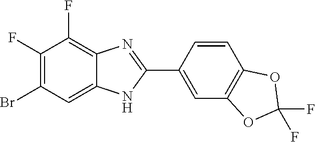 [SMILES: Fc1c(Br)cc2[nH]c(-c3ccc4c(c3)OC(F)(F)O4)nc2c1F] | 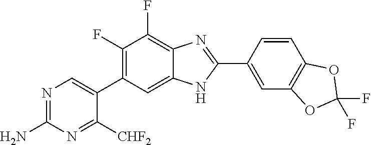 [SMILES: Nc1ncc(-c2cc3[nH]c(-c4ccc5c(c4)OC(F)(F)O5)nc3c(F)c2F)c(C(F)F)n1] | 5-(2-(2,2-difluorobenzo[d][1,3] dioxol-5-yl)-4,5-difluoro-1H- benzo[d]imidazol-6-yl)-4- (difluoromethyl)pyrimidine-2- amine | 454.3 |
| 90 | 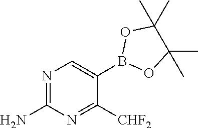 [SMILES: CC1(C)OB(c2cnc(N)nc2C(F)F)OC1(C)C] | 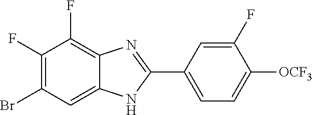 [SMILES: Fc1cc(-c2nc3c(F)c(F)c(Br)cc3[nH]2)ccc1OC(F)(F)F] | 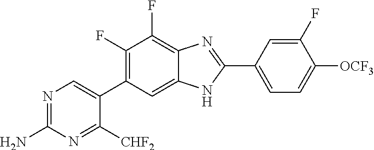 [SMILES: Nc1ncc(-c2cc3[nH]c(-c4ccc(OC(F)(F)F)c(F)c4)nc3c(F)c2F)c(C(F)F)n1] | 5-difluoro-1H-benzo[d]5-(2- (4-(trifluoromethoxy)-3-fluoro- phenyl)-4,5-difluoro-1H-benzo [d]imidazol-6-yl)-4-(difluoro- methyl)pyrimidine-2-amine | 476.1 |
| 91 | 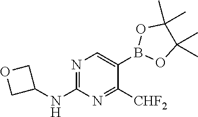 [SMILES: CC1(C)OB(c2cnc(NC3COC3)nc2C(F)F)OC1(C)C] | 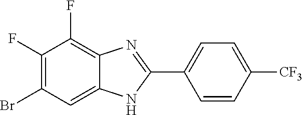 [SMILES: Fc1c(Br)cc2[nH]c(-c3ccc(C(F)(F)F)cc3)nc2c1F] | 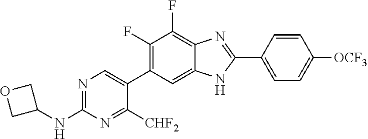 [SMILES: Fc1c(-c2cnc(NC3COC3)nc2C(F)F)cc2[nH]c(-c3ccc(OC(F)(F)F)cc3)nc2c1F] | 5-(2-(4-(trifluoromethyl)phen- yl)-4,5-difluoro-1H-benzo[d] imidazol-6-yl)-4-(difluoro- methyl)-N-(oxetan-3-yl)pyr- imidine-2-amine | 498.4 |
| 92 | 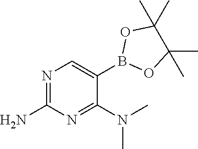 [SMILES: CN(C)c1nc(N)ncc1B1OC(C)(C)C(C)(C)O1] | 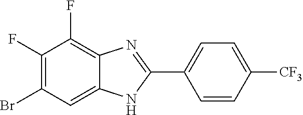 [SMILES: Fc1c(Br)cc2[nH]c(-c3ccc(C(F)(F)F)cc3)nc2c1F] | 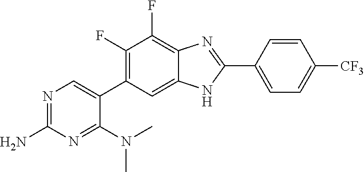 [SMILES: CN(C)c1nc(N)ncc1-c1cc2[nH]c(-c3ccc(C(F)(F)F)cc3)nc2c(F)c1F] | 5-(2-(4-(trifluoromethyl)phen- yl)-4,5-difluoro-1H-benzo[d] imidazol-6-yl)-N4,N4-dimeth- ylpyrimidine-2,4-diamine | 435.1 |
| 93 | 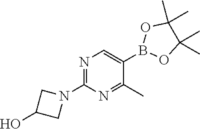 [SMILES: Cc1nc(N2CC(O)C2)ncc1B1OC(C)(C)C(C)(C)O1] | 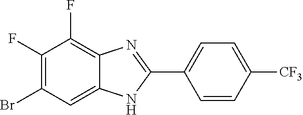 [SMILES: Fc1c(Br)cc2[nH]c(-c3ccc(C(F)(F)F)cc3)nc2c1F] | 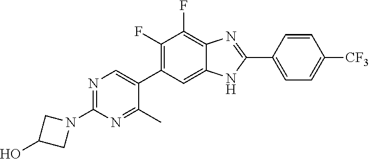 [SMILES: Cc1nc(N2CC(O)C2)ncc1-c1cc2[nH]c(-c3ccc(C(F)(F)F)cc3)nc2c(F)c1F] | 1-(4-methyl-5-(2-(4-(trifluoro- methyl)phenyl)-4,5-difluoro- 1H-benzo[d]imidazol-6-yl)- pyrimidine-2-yl)azetidin-3-ol | 462.1 |
| 94 | 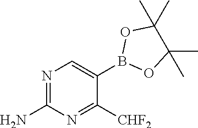 [SMILES: CC1(C)OB(c2cnc(N)nc2C(F)F)OC1(C)C] | 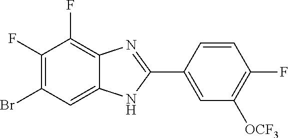 [SMILES: Fc1ccc(-c2nc3c(F)c(F)c(Br)cc3[nH]2)cc1OC(F)(F)F] | 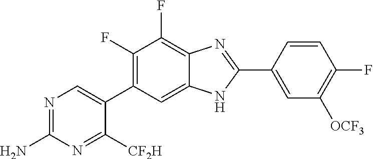 [SMILES: Nc1ncc(-c2cc3[nH]c(-c4ccc(F)c(OC(F)(F)F)c4)nc3c(F)c2F)c(C(F)F)n1] | 5-(2-(3-(trifluoromethoxy)-4- fluorophenyl)-4,5-difluoro- 1H-benzo[d]imidazol-6-yl)-4- (difluoromethyl)pyrimidine- 2-amine | 476.1 |
| 95 | 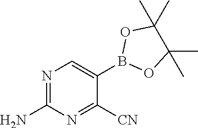 [SMILES: CC1(C)OB(c2cnc(N)nc2C#N)OC1(C)C] | 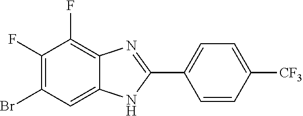 [SMILES: Fc1c(Br)cc2[nH]c(-c3ccc(C(F)(F)F)cc3)nc2c1F] | 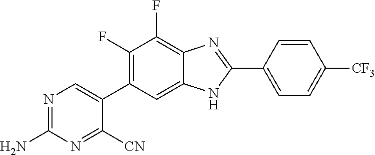 [SMILES: N#Cc1nc(N)ncc1-c1cc2[nH]c(-c3ccc(C(F)(F)F)cc3)nc2c(F)c1F] | 2-amino-5-(2-(4-(trifluoro- methyl)phenyl)-4,5-difluoro- 1H-benzo[d]imidazol-6-yl)- pyrimidine-4-carbonitrile | 417.1 |
| 96 | 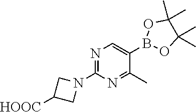 [SMILES: Cc1nc(N2CC(C(=O)O)C2)ncc1B1OC(C)(C)C(C)(C)O1] | 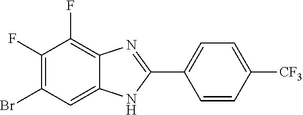 [SMILES: Fc1c(Br)cc2[nH]c(-c3ccc(C(F)(F)F)cc3)nc2c1F] | 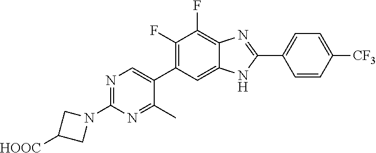 [SMILES: Cc1nc(N2CC(C(=O)O)C2)ncc1-c1cc2[nH]c(-c3ccc(C(F)(F)F)cc3)nc2c(F)c1F] | 1-(4-methyl-5-(2-(4-(trifluoro- methyl)phenyl)-4,5-difluoro- 1H-benzo[d]imidazol-6-yl)- pyrimidine-2-yl)azetidine-3- carboxylic acid | 490.5 |
| 97 | 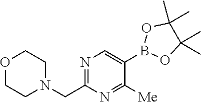 [SMILES: Cc1nc(CN2CCOCC2)ncc1B1OC(C)(C)C(C)(C)O1] | 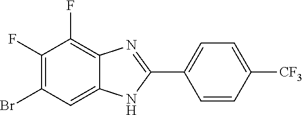 [SMILES: Fc1c(Br)cc2[nH]c(-c3ccc(C(F)(F)F)cc3)nc2c1F] | 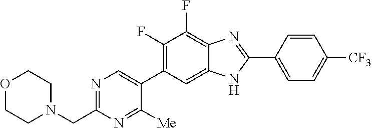 [SMILES: Cc1nc(CN2CCOCC2)ncc1-c1cc2[nH]c(-c3ccc(C(F)(F)F)cc3)nc2c(F)c1F] | 4-((4-methyl-5-(2-(4-(trifluoro- methyl)phenyl)-4,5-difluoro- 1H-benzo[d]imidazol-6-yl)- pyrimidine-2-yl)methyl)mor- pholine | 490.2 |
| 98 | 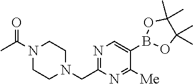 [SMILES: CC(=O)N1CCN(Cc2ncc(B3OC(C)(C)C(C)(C)O3)c(C)n2)CC1] | 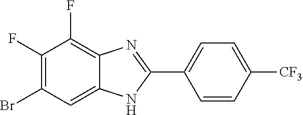 [SMILES: Fc1c(Br)cc2[nH]c(-c3ccc(C(F)(F)F)cc3)nc2c1F] | 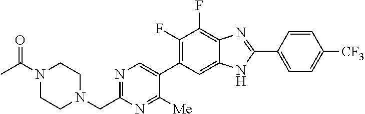 [SMILES: CC(=O)N1CCN(Cc2ncc(-c3cc4[nH]c(-c5ccc(C(F)(F)F)cc5)nc4c(F)c3F)c(C)n2)CC1] | 1-(4-((4-methyl-5-(2-(4- (trifluoromethyl)phenyl)-4,5- difluoro-1H-benzo[d]imid- azol-6-yl)pyrimidine-2-yl)- methyl)piperazin-1-yl)ethan- 1-one | 531.2 |
| 99 | 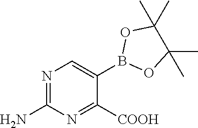 [SMILES: CC1(C)OB(c2cnc(N)nc2C(=O)O)OC1(C)C] | 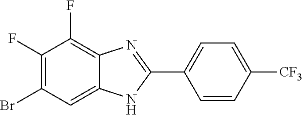 [SMILES: Fc1c(Br)cc2[nH]c(-c3ccc(C(F)(F)F)cc3)nc2c1F] | 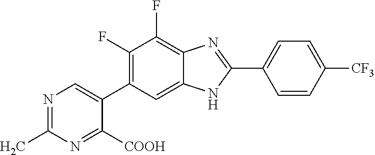 [SMILES: Cc1ncc(-c2cc3[nH]c(-c4ccc(C(F)(F)F)cc4)nc3c(F)c2F)c(C(=O)O)n1] | 2-amino-5-(2-(4-(trifluoro- methyl)phenyl)-4,5-difluoro- 1H-benzo[d]imidazol-6-yl)- pyrimidine-4-carboxylic acid | 436.1 |
| 100 | 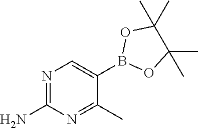 [SMILES: Cc1nc(N)ncc1B1OC(C)(C)C(C)(C)O1] | 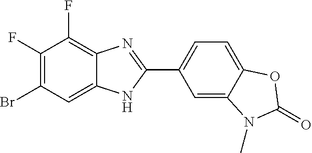 [SMILES: Cn1c(=O)oc2ccc(-c3nc4c(F)c(F)c(Br)cc4[nH]3)cc21] | 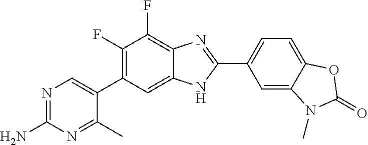 [SMILES: Cc1nc(N)ncc1-c1cc2[nH]c(-c3ccc4oc(=O)n(C)c4c3)nc2c(F)c1F] | 3-methyl-5-(6-(4-methyl-2- aminopyrimid-5-yl)-4,5- difluoro-1H-benzo[d]imid- azol-2-yl)benzo[d]oxazol- 2(3H)-one | 409.2 |
| 101 | 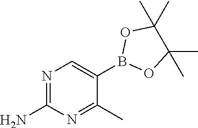 [SMILES: Cc1nc(N)ncc1B1OC(C)(C)C(C)(C)O1] | 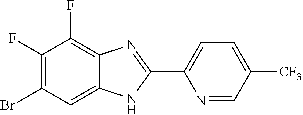 [SMILES: Fc1c(Br)cc2[nH]c(-c3ccc(C(F)(F)F)cn3)nc2c1F] | 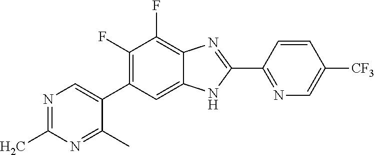 [SMILES: Cc1ncc(-c2cc3[nH]c(-c4ccc(C(F)(F)F)cn4)nc3c(F)c2F)c(C)n1] | 4-methyl-5-(2-(5-(trifluoro- methyl)pyrid-2-yl)-4,5- difluoro-1H-benzo[d]imid- azol-6-yl)pyrimidine-2-amine | 407.0 |
| 102 | 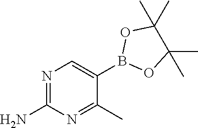 [SMILES: Cc1nc(N)ncc1B1OC(C)(C)C(C)(C)O1] | 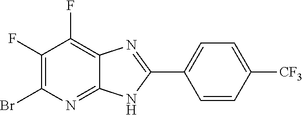 [SMILES: Fc1c(Br)nc2[nH]c(-c3ccc(C(F)(F)F)cc3)nc2c1F] | 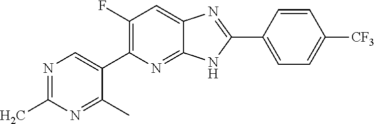 [SMILES: Cc1ncc(-c2nc3[nH]c(-c4ccc(C(F)(F)F)cc4)nc3cc2F)c(C)n1] | 4-methyl-5-(2-(4-(trifluoro methyl) phenyl)-6-fluoro-3H- imidazo[4,5-b]pyridin-5-yl)- pyrimidine-2-amine | 389.2 |
| 103 | 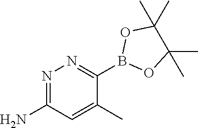 [SMILES: Cc1cc(N)nnc1B1OC(C)(C)C(C)(C)O1] | 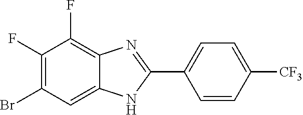 [SMILES: Fc1c(Br)cc2[nH]c(-c3ccc(C(F)(F)F)cc3)nc2c1F] | 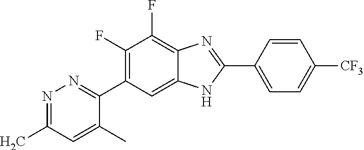 [SMILES: Cc1cc(C)c(-c2cc3[nH]c(-c4ccc(C(F)(F)F)cc4)nc3c(F)c2F)nn1] | 5-methyl-6-(2-(4-(trifluoro- methyl)phenyl)-4,5-difluoro- 1H-benzo[d]imidazol-6-yl)- pyridazin-3-amine | 406.2 |
| 104 | 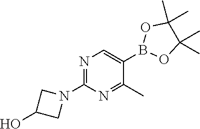 [SMILES: Cc1nc(N2CC(O)C2)ncc1B1OC(C)(C)C(C)(C)O1] | 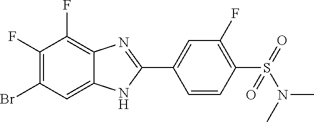 [SMILES: CN(C)S(=O)(=O)c1ccc(-c2nc3c(F)c(F)c(Br)cc3[nH]2)cc1F] | 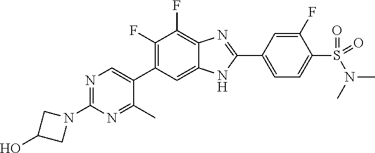 [SMILES: Cc1nc(N2CC(O)C2)ncc1-c1cc2[nH]c(-c3ccc(S(=O)(=O)N(C)C)c(F)c3)nc2c(F)c1F] | 4-(6-(4-methyl-2-(3-hydroxy- azetidin-1-yl)pyrimid-5-yl)- 4,5-difluoro-1H-benzo[d] imidazol-2-yl)-2-fluoro-N,N- dimethylbenzenesulfonamide | 519.1 |
| 105 | 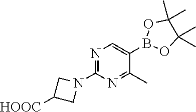 [SMILES: Cc1nc(N2CC(C(=O)O)C2)ncc1B1OC(C)(C)C(C)(C)O1] | 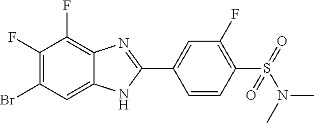 [SMILES: CN(C)S(=O)(=O)c1ccc(-c2nc3c(F)c(F)c(Br)cc3[nH]2)cc1F] | 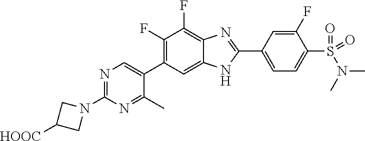 [SMILES: Cc1nc(N2CC(C(=O)O)C2)ncc1-c1cc2[nH]c(-c3ccc(S(=O)(=O)N(C)C)c(F)c3)nc2c(F)c1F] | 1-(4-methyl-5-(2-(3-fluoro-4- (N,N-dimethylsulfamoyl)phen- yl)-4,5-difluoro-1H-benzo[d] imidazol-6-yl)pyrimidine-2- yl)azetidine-3-carboxylic acid | 547.5 |

TABLE 2
Nuclear magnetic resonance characterization data of compounds 1-105 in Example 1

| Compound | |
|---|---|
| No. | Nuclear magnetic data |
| 1 | ¹H NMR (400 MHz, DMSO-d₆): δ 8.47 (d, J = 4.8 Hz, 1H), 8.40 (d, J = 8.4 |
| | Hz, 2H), 7.96 (d, J = 4.8 Hz, 1H), 7.91 (d, J = 8.4 Hz, 2H), 7.71-7.53 (m, 3H), |
| | 2.65 (s, 6H) |
| 2 | ¹H NMR (400 MHz, DMSO-d₆): δ 13.09 (br s, 1H), 8.75 (s, 1H), 8.60 (d, J = |
| | 4.4 Hz, 1H), 7.98 (s, 1H), 7.96 (d, J = 3.6 Hz, 1H) 7.56-7.51 (m, 3 H), 7.35 (t, |
| | J = 4.8 Hz, 1H), 3.90 (s, 3H) |
| 3 | ¹H NMR (400 MHz, DMSO-d₆): δ 8.20 (d, J = 8.4 Hz, 2H), 7.81 (d, J = 7.6 |
| | Hz, 1H), 7.53 (d, J = 8.0 Hz, 4H), 7.37 (d, J = 7.6 Hz, 1H), 6.87 (m, 2H), |
| | 4.47-4.6 (m, 1H), 3.60-3.40 (m, 2H), 2.98-2.82 (m, 2H), 1.98-1.86 (m, 2H), |
| | 1.65-1.42 (m, 2H) |
| 4 | ¹H NMR (400 MHz, DMSO-d₆): δ 8.47 (s, 1H), 8.28 (d, J = 8 Hz, 2H), 7.88 |
| | (d, J = 8 Hz, 2H), 7.66 (s, 1H), 7.51 (br s, 1H), 3.23 (s, 3H), 2.73 (s, 3H) |
| 5 | ¹H NMR (400 MHz, DMSO-d₆): δ 8.54 (s, 1H), 8.41 (d, J = 8.0 Hz, 2H), 7.94 |
| | (d, J = 8.4 Hz, 2H), 7.72 (br s, 1H), 2.68 (s, 3H), 2.30 (s, 3H) |
| 6 | ¹H NMR (400 MHz, DMSO-d₆): δ 8.86 (s, 2H), 8.40 (d, J = 4.4 Hz, 2H), 7.94 |
| | (d, J = 8.4 Hz, 2H), 7.68 (br s, 1H), 2.69 (s, 3H) |
| 7 | ¹H NMR (400 MHz, DMSO-d₆): δ 8.35 (s, 1H), 8.33 (d, J = 8.0 Hz, 2H), 7.87 |
| | (d, J = 8.4 Hz, 2H), 7.63-7.56 (m, 2H), 6.28 (br s, 1H), 2.98 (s, 3H), 2.61 (s, |
| | 3H) |
| 8 | ¹H NMR (400 MHz, DMSO-d₆): δ 8.36 (d, J = 8.0 Hz, 2H), 8.05 (s, 1H), 7.91 |
| | (d, J = 8.0 Hz, 2H), 7.58 (s, 1H), 7.48 (br s, 2H), 2.12 (s, 3H) |
| 9 | ¹H NMR (400 MHz, DMSO-d₆): δ 8.55 (s, 2H), 8.35 (d, J = 8.0 Hz, 2H), 7.91 |
| | (d, J = 8.0 Hz, 2H), 7.49 (br s, 1H), 3.15 (s, 6H) |
| 10 | ¹H NMR (400 MHz, DMSO-d₆): δ 8.54 (s, 1 H), 8.47 (s, 1H), 8.38 (d, J = 8.0 |
| | Hz, 2H), 7.92 (d, J = 8.0 Hz, 2H), 7.60-7.54 (m, 2H), 7.30 (d, J = 4.4 Hz, 1H), |
| | 2.16 (s, 3H) |
| 11 | ¹H NMR (400 MHz, DMSO-d₆): δ 8.51 (s, 1 H), 8.46 (s, 1H), 8.35 (d, J = 8.0 |
| | Hz, 2H), 7.90 (d, J = 8.0 Hz, 2H), 7.63-7.54 (m, 2H), 7.30 (d, J = 4.4 Hz, 1H), |
| | 7.18 (t, J = 61.2 Hz, 1H), 2.16 (s, 3H) |
| 12 | ¹H NMR (400 MHz, DMSO-d₆): δ 8.37 (d, J = 8.0 Hz, 2H), 7.92 (d, J = 8.0 |
| | Hz, 2H), 7.81 (d, J = 8.0 Hz, 2H), 7.60-7.54 (m, 2H), 7.38 (d, J = 8.4 Hz, 1H), |
| | 2.46 (s, 3H) |
| 13 | ¹H NMR (400 MHz, DMSO-d₆): δ 8.36 (d, J = 8.0 Hz, 2H), 7.91 (d, J = 8.0 |
| | Hz, 2H), 7.57-7.46 (m, 3 H), 3.81 (s, 3H), 2.56 (s, 3H) |
| 14 | ¹H NMR (400 MHz, DMSO-d₆): δ 8.92 (s, 2H), 8.41 (d, J = 8.0 Hz, 2H), 7.93 |
| | (d, J = 8.0 Hz, 2H), 7.55 (d, J = 8.4 Hz, 1H), 7.43 (d, J = 7.6 Hz, 1H), 2.66 (s, |
| | 3H) |
| 15 | ¹H NMR (400 MHz, DMSO-d₆): δ 8.62 (s, 1H), 8.36 (d, J = 8.0 Hz, 2H), 7.92 |
| | (d, J = 8.0 Hz, 2H), 7.88 (d, J = 7.6 Hz, 1H), 7.67 (d, J = 7.6 Hz, 1H), 7.55 (d, |
| | J = 8.0 Hz, 1H), 7.39 (d, J = 7.6 Hz, 1H), 2.47 (s, 3H) |
| 16 | ¹H NMR (400 MHz, DMSO-d₆): δ 13.37 (br s, 1H), 8.60 (s, 2H), 8.36 (d, J = |
| | 8.0 Hz, 2H), 7.91 (d, J = 8.0 Hz, 2H), 7.71 (m, 1H), 7.54 (m, 1H), 3.74 (t, J = |
| | 4.0 Hz, 4H), 3.66 (t, J = 8.0 Hz, 4H) |
| 17 | ¹H NMR (400 MHz, DMSO-d₆): δ 8.57 (s, 2H), 8.36 (d, J = 7.6 Hz, 2H), 7.91 |
| | (d, J = 7.2 Hz, 2H), 7.71 (br s, 1H), 7.59-7.51 (m, 2H), 3.76 (m, 4H), 2.35 (m, |
| | 4H), 2.19 (s, 3H) |
| 18 | ¹H NMR (400 MHz, DMSO-d₆): δ 13.30 (br s, 1H), 8.47 (s, 1H), 8.18 (d, J = |
| | 7.6 Hz, 2H), 7.95 (t, J = 6.4 Hz, 1H), 7.69 (d, J = 7.6 Hz, 2H), 7.62-7.46 (m, |
| | 3H), 3.48-3.40 (m, 4H), 1.88-1.78 (m, 4H) |
| 19 | ¹H NMR (400 MHz, DMSO-d₆): δ 8.40 (d, J = 8.0 Hz, 2H), 8.1 (d, J = 8.4 |
| | Hz, 2H), 7.60 (m, 2H), 7.5 (d, J = 1.8 Hz, 1H), 6.32 (d, J = 1.8 Hz, 1H), 4.31 |
| | (m, 1H), 1.31 (d, J = 6.4 Hz, 6H) |
| 20 | ¹H NMR (400 MHz, DMSO-d₆): δ 8.61 (s, 2H), 8.40 (d, J = 8.4 Hz, 2H), 8.08 |
| | (d, J = 8.4 Hz, 2H), 7.72 (br, 1H), 7.56 (m, 1H), 3.75 (m, 4H), 3.67 (q, J = 4.4 |
| | Hz, 4H), 3.26 (s, 3H) |
| 21 | ¹H NMR (400 MHz, DMSO-d₆): δ 8.40 (d, J = 8.4 Hz, 2H), 7.92 (d, J = 8.4 |
| | Hz, 2H), 7.60-7.56 (m, 2H), 7.60 (s, 1H), 6.32 (d, J = 1.6 Hz, 1H), 4.95 (m, |
| | 1H), 2.64 (s, 6H), 1.40 (d, J = 6.4 Hz, 6H) |
| 22 | ¹H NMR (400 MHz, DMSO-d₆): δ 8.39 (d, J = 8.0 Hz, 2H), 8.05 (s, 1H), 7.91 |
| | (d, J = 8.0 Hz, 2H), 7.50-7.42 (m, 2H), 6.65 (s, 2H), 2.59 (s, 6H), 2.13 (s, 3H) |
| 23 | ¹H NMR (400 MHz, DMSO-d₆): δ 8.36 (d, J = 8.0 Hz, 2H), 7.92 (d, J = 8.0 |
| | Hz, 2H), 7.55 (m, 2H), 2.62 (s, 3H), 2.38 (s, 3H), 2.12 (s, 3H) |
| 24 | ¹H NMR (400 MHz, DMSO-d₆): δ 8.36 (d, J = 8.0 Hz, 2H), 7.92 (d, J = 8.0 |
| | Hz, 2H), 7.55 (m, 2H), 3.53 (s, 3H), 2.34 (s, 3H), 2.12 (s, 3H) |
| 25 | ¹H NMR (400 MHz, DMSO-d₆): δ 8.41 (d, J = 8.4 Hz, 2H), 7.90 (d, J = 8.4 |
| | Hz, 2H), 7.60 (s, 1H), 7.56 (d, J = 1.6 Hz, 1H), 6.33 (d, J = 1.6 Hz, 1H), 4.98 |
| | (m, 1H), 2.63 (s, 6H), 1.39 (d, J = 6.4 Hz, 6H) |
| 26 | ¹H NMR (400 MHz, DMSO-d₆): δ 8.42 (d, J = 8.0 Hz, 2H), 8.09 (s, 1H), 7.98 |
| | (d, J = 8.0 Hz, 2H), 7.54(m, 2H), 3.76 (m, 4H), 3.65 (q, J = 4.4 Hz, 4H), 2.63 |
| | (s, 6H), 2.15 (s, 3H) |
| 27 | ¹H NMR (400 MHz, DMSO-d₆): δ 8.40 (d, J = 8.4 Hz, 2H), 8.07 (s, 1H), 7.92 |
| | (d, J = 8.4 Hz, 2H), 7.51 (s, 1H), 6.67 (s, 2H), 2.59 (s, 6H), 2.15 (s, 3H) |
| 28 | ¹H NMR (400 MHz, DMSO-d₆): δ 8.71 (s, 1H), 8.41 (d, J = 8.4 Hz, 2H), 7.90 |
| | (d, J = 8.4 Hz, 2H), 7.61(s, 1H), 2.74 (s, 3H), 2.62 (s, 6H) |
| 29 | ¹H NMR (400 MHz, DMSO-d₆): δ 8.51 (s, 1H), 8.40 (d, J = 8.4 Hz, 2H), 7.92 |
| | (d, J = 8.4 Hz, 2H), 7.59 (m, 2H), 2.64 (s, 6H), 2.60 (s, 3H), 2.31 (s, 3H) |
| 30 | ¹H NMR (400 MHz, DMSO-d₆): δ 8.65 (s, 1H), 8.40 (d, J = 8.4 Hz, 2H), 8.29 |
| | (d, J = 8.4 Hz, 2H), 7.63 (s, 1H), 7.51 (s, 1H), 2.73 (s, 3H), 2.62 (6H) |
| 31 | ¹H NMR (400 MHz, DMSO-d₆): δ 8.41 (d, J = 8.4 Hz, 2H), 8.05 (s, 1H), 7.90 |
| | (d, J = 8.4 Hz, 2H), 7.48 (s, 1H), 6.67 (s, 2H), 2.63 (s, 6H), 2.16 (s, 3H) |
| 32 | ¹H NMR (400 MHz, DMSO-d₆): δ 8.61 (s, 1H), 8.39 (d, J = 8.0 Hz, 2H), 7.92 |
| | (d, J = 8.0 Hz, 2H), 7.56 (m, 2H), 4.01 (s, 3H), 2.63 (s, 6H), 2.61 (s, 3H) |
| 33 | ¹H NMR (400 MHz, DMSO-d₆): δ 9.34 (s, 1H), 9.12 (s, 1H), 8.42 (d, J = 7.6 |
| | Hz, 2H), 7.92 (d, J = 7.6 Hz, 2H), 7.68 (s, 1H), 7.62 (s, 1H), 2.60 (s, 6H) |
| 34 | ¹H NMR (400 MHz, DMSO-d₆): δ 8.41 (d, J = 8.4 Hz, 2H), 8.07 (s, 1H), 7.90 |
| | (d, J = 8.0 Hz, 2H), 7.53-7.46 (m, 2H), 6.62 (br s, 2H), 2.62 (s, 6H), 2.38 (m, |
| | 1H), 1.36 (d, J = 1.2 Hz, 6H) |
| 35 | ¹H NMR (400 MHz, DMSO-d₆): δ 8.39 (d, J = 8.4 Hz, 2H), 8.06 (s, 1H), 7.90 |
| | (d, J = 8.4 Hz, 2H), 7.45(s, 1H), 6.62 (s, 2H), 2.63 (s, 6H), 2.15 (s, 3H) |
| 36 | ¹H NMR (400 MHz, DMSO-d₆): δ 8.32 (d, J = 8.0 Hz, 2H), 8.14 (s, 1H), 7.74 |
| | (d, J = 8.0 Hz, 2H), 7.50 (d, J = 1.2 Hz, 1H), 7.47 (d, J = 1.2 Hz, 1H), 6.97 (s, |
| | 1H), 6.38 (br s, 2H), 2.30 (s, 3H) |
| 37 | ¹H NMR (400 MHz, DMSO-d₆): δ 8.37 (d, J = 8.4 Hz, 2H), 8.12(s, 1H), 7.73 |
| | (d, J = 8.4 Hz, 2H), 7.50 (d, J = 1.2 Hz, 1H), 7.46 (d, J = 1.2 Hz, 1H), 7.01 (s, |
| | 1H), 5.94 (br, 1H), 2.58 (s, 3H), 2.32 (s, 3H) |
| 38 | ¹H NMR (400 MHz, DMSO-d₆): δ 8.43 (d, J = 8.4 Hz, 2H), 7.91 (d, J = 8.4 |
| | Hz, 2H), 7.63-7.56 (m, 2H), 7.60 (s, 1H), 6.32 (d, J = 1.6 Hz, 1H), 2.63 (s, |
| | 6H), 1.43 (d, J = 6.4 Hz, 6H) |
| 39 | ¹H NMR (400 MHz, DMSO-d₆): δ 8.38 (d, J = 8.0 Hz, 2H), 8.0 (s, 1H), 7.99 |
| | (d, J = 8.0 Hz, 2H), 7.93-7.91 (m, 2H), 6.79 (s, 1H), 3.88 (s, 3H), 2.62 (s, |
| | 6H), 2.11 (s, 3H) |
| 40 | ¹H NMR (400 MHz, DMSO-d₆): δ 8.36 (d, J = 8.0 Hz, 2H), 7.82 (d, J = 8.0 |
| | Hz, 2H), 7.50 (d, J = 8.0 Hz, 1H), 7.42 (d, J = 8.0 Hz, 1H), 7.38 (s, 1H), 7.26 |
| | (s, 1H), 6.48 (s, 2H), 2.60 (s, 6H) |
| 41 | ¹H NMR (400 MHz, DMSO-d₆): δ 11.80 (br s, 1H), 8.35 (d, J = 8.0 Hz, 2H), |
| | 7.92 (d, J = 8.0 Hz, 2H), 7.51-7.48 (m, 2H), 7.34 (d, J = 8.0 Hz, 1H), 6.22 (d, |
| | J = 8.0 Hz, 1H), 2.59 (s, 6H), 2.05 (s, 3H) |
| 42 | ¹H NMR (400 MHz, DMSO-d₆): δ 8.38 (d, J = 8.0 Hz, 2H), 8.08 (s, 1H), 7.94 |
| | (d, J = 8.0 Hz, 2H), 7.54 (m, 2H), 6.67 (s, 2H), 2.16 (s, 3H) |
| 43 | ¹H NMR (400 MHz, DMSO-d₆) δ 13.30 (s, 1H), 8.55 (s, 1H), 8.40 (d, J = 8.0 |
| | Hz, 2H), 7.96 (d, J = 8.2 Hz, 2H), 7.55 (m, 2H), 2.65 (s, 3H), 2.34 (s, 3H) |
| 44 | ¹H NMR (400 MHz, DMSO-d₆) δ 13.37 (s, 1H), 8.34 (d, J = 8.0 Hz, 2H), |
| | 8.11 (s, 1H), 7.78 (d, J = 8.0 Hz, 2H), 7.33 (s, 1H), 7.13 (t, J = 57.6 Hz, 1H), |
| | 6.75 (s, 2H), 2.13 (s, 3H) |
| 45 | ¹H NMR (400 MHz, DMSO-d₆) δ 13.67 (s, 1H), 8.46 (d, J = 8.4 Hz, 2H), |
| | 8.15 (s, 1H, HCOOH), 7.96 (d, J = 8.4 Hz, 2H), 7.80 (s, 1H), 7.26 (s, 1H), |
| | 6.41 (s, 1H), 6.04 (s, 2H), 2.68 (s, 6H), 2.05 (s, 3H) |
| 46 | ¹H NMR (400 MHz, DMSO-d₆) δ 13.40 (s, 1H), 8.43 (s, 1H), 8.36 (d, J = 8.0 |
| | Hz, 2H), 7.79 (d, J = 8.0 Hz, 2H), 7.37 (s, 1H), 7.31 (s, 2H), 7.14 (t, J = 55.6 |
| | Hz, 1H), 6.74 (t, J = 53.2 Hz, 1H) |
| 47 | ¹H NMR (400 MHz, DMSO-d₆) δ 13.26 (s, 1H), 9.21 (s, 1H), 8.68 (s, 1H), |
| | 8.44 (d, J = 8.4 Hz, 2H), 7.95 (d, J = 8.2 Hz, 2H), 7.76-7.57 (m, 2H), 3.02- |
| | 2.96 (m, 1H), 2.68 (s, 6H), 1.23-1.16 (m, 6H) |
| 48 | ¹H NMR (400 MHz, DMSO-d₆) δ 13.37 (s, 1H), 8.42 (d, J = 8.4 Hz, 2H), |
| | 8.14 (s, 1H, HCOOH), 7.94 (d, J = 8.4 Hz, 2H), 7.76 (s, 1H), 7.57 (s, 1H), |
| | 7.46-7.39 (m, 1H), 6.40 (s, 1H), 5.95 (s, 2H), 2.68 (s, 6H), 2.04 (s, 3H) |
| 49 | ¹H NMR (400 MHz, DMSO-d₆) δ 13.44 (s, 1H), 8.43 (d, J = 8.4 Hz, 2H), |
| | 7.96-7.93 (m, 3H), 7.57 (m, 2H), 6.76 (s, 1H), 6.71(t, J = 54.4 Hz, 1H), 6.46 |
| | (s, 2H), 2.68 (s, 6H) |
| 50 | ¹H NMR (400 MHz, DMSO-d₆) δ 13.44 (s, 1H), 8.42 (d, J = 8.4 Hz, 2H), |
| | 8.00-7.88 (m, 3H), 7.57 (m, 2H), 3.88 (s, 3H), 2.84 (s, 6H), 2.68 (s, 6H) |
| 51 | ¹H NMR (400 MHz, DMSO-d₆) δ 8.50-8.47 (m, 2H), 8.41 (s, 1H), 8.03-7.96 |
| | (m, 2H), 7.71-7.65 (m, 2H), 6.63 (t, J = 53.2 Hz, 1H), 2.69 (s, 6H) |
| 52 | ¹H NMR (400 MHz, DMSO-d₆) δ 13.40 (s, 1H), 8.42 (d, J = 8.4 Hz, 2H), |
| | 7.99 (s, 1H), 7.93 (d, J = 8.4 Hz, 2H), 7.52-7.50 (m, 2H), 6.86 (s, 1H), 6.66 (s, |
| | 2H), 2.67 (s, 6H) |
| 53 | ¹H NMR (400 MHz, DMSO-d₆) δ 13.56 (s, 1H), 8.44 (d, J = 8.4 Hz, 2H), |
| | 8.13 (s, 1H), 7.95 (d, J = 8.4 Hz, 2H), 7.62 (m, 2H), 7.23 (s, 1H), 2.68 (s, 6H), |
| | 2.24 (s, 3H) |
| 54 | ¹H NMR (400 MHz, DMSO-d₆) δ 13.54 (s, 1H), 8.85 (d, J = 5.2 Hz, 1H), |
| | 8.72 (s, 1H), 8.45 (d, J = 8.4 Hz, 2H), 7.96 (d, J = 8.4 Hz, 2H), 7.77 (d, J = |
| | 5.2 Hz, 1H), 7.67-7.59 (m, 2H), 6.93 (t, J = 54.0 Hz, 1H), 2.68 (s, 6H) |
| 55 | ¹H NMR (400 MHz, DMSO-d₆) δ 13.49 (s, 1H), 8.50 (d, J = 5.0 Hz, 1H), |
| | 8.47-8.41 (m, 3H), 7.99-7.90 (m, 2H), 7.62 (m, 2H), 7.40 (d, J = 5.0 Hz, 1H), |
| | 2.68 (s, 6H), 2.21 (s, 3H) |
| 56 | ¹H NMR (400 MHz, DMSO-d₆) δ 13.55 (s, 1H), 8.95 (d, J = 5.2 Hz, 1H), |
| | 8.81 (s, 1H), 8.49-8.40 (m, 2H), 8.00-7.90 (m, 3H), 7.66 (s, 2H), 2.69 (s, 6H) |
| 57 | ¹H NMR (400 MHz, DMSO-d₆) δ 13.73 (s, 1H), 8.52-8.39 (m, 2H), 8.02-7.90 |
| | (m, 3H), 7.37 (d, J = 5.1 Hz, 1H), 6.67 (s, 1H), 6.49 (s, 2H), 2.68 (s, 6H) |
| 58 | ¹H NMR (400 MHz, DMSO-d₆) δ 13.46 (s, 1H), 8.43 (d, J = 8.0 Hz, 2H), |
| | 8.29 (s, 1H), 7.94 (d, J = 8.0 Hz, 2H), 7.64 (s, 1H), 7.57 (d, J = 10.0 Hz, 1H), |
| | 7.29 (s, 2H), 2.67 (s, 6H) |
| 59 | ¹H NMR (400 MHz, DMSO-d₆) δ 13.44 (s, 1H), 8.39 (d, J = 8.0 Hz, |
| | 2H), 8.28 (s, 1H), 7.95 (d, J = 8.0 Hz, 2H), 7.71-7.48 (m, 2H), 7.29 (s, |
| | 2H) |
| 60 | ¹H NMR (400 MHz, DMSO-d6) δ 13.51 (s, 1H), 8.43 (d, J = 8.4 Hz, |
| | 2H), 7.95 (d, J = 8.4 Hz, 2H), 7.59 (s, 2H), 5.52 (s, 1H), 4.69 (s, 2H), |
| | 4.07 (p, J = 6.8 Hz, 1H), 2.68 (s, 6H), 1.27 (d, J = 6.4 Hz, 6H) |
| 61 | ¹H NMR (400 MHz, DMSO-d₆) δ 13.32 (s, 1H), 8.39 (d, J = 8.4 Hz, 2H), |
| | 7.96-7.94 (m, 3H), 7.44 (m, 2H), 6.65 (s, 1H), 6.39 (s, 2H) |
| 62 | 1H NMR (400 MHz, DMSO-d6) δ 13.23 (s, 1H), 8.44 (d, J = 8.4 Hz, 2H), |
| | 7.94 (d, J = 8.4 Hz, 2H), 7.62-7.59 (m, 2H), 5.59 (s, 1H), 4.64 (s, 2H), 3.49(s, |
| | 3H), 2.67 (s, 6H) |
| 63 | ¹H NMR (400 MHz, DMSO-d₆) δ 13.49 (s, 1H), 8.46 (s, 1H), 8.43 (d, J = 8.0 |
| | Hz, 2H), 7.95 (d, J = 8.0 Hz, 2H), 7.65 (d, J = 6.0 Hz, 1H), 7.58 (d, J = 10.4 |
| | Hz, 1H), 7.06 (s, 2H), 2.67 (s, 6H) |
| 64 | ¹H NMR (400 MHz, DMSO-d₆) δ 13.48 (s, 1H), 8.46 (s, 1H), 8.39 (d, J = 8.0 |
| | Hz, 2H), 7.96 (d, J = 8.0 Hz, 2H), 7.64-7.56 (m, 2H), 7.04 (s, 2H) |
| 65 | ¹H NMR (400 MHz, DMSO-d₆) δ 13.53 (s, 1H), 8.42-8.40 (m, 3H), 7.97 (d, J = |
| | 8.4 Hz, 2H), 7.81-7.53 (m, 3H), 6.95 (t, J = 53.6 Hz, 1H) |
| 66 | ¹H NMR (400 MHz, DMSO-d₆) δ 13.50 (s, 1H), 9.44 (s, 1H), 9.07 (s, 1H), |
| | 8.41 (d, J = 8.0 Hz, 2H), 7.97 (d, J = 8.0 Hz, 2H), 7.77 (d, J = 6.4 Hz, 1H), |
| | 7.66 (d, J = 10.0 Hz, 1H), 6.94 (t, J = 52.8 Hz, 1H) |
| 67 | ¹H NMR (400 MHz, DMSO-d₆) δ 13.48 (s, 1H), 9.26 (d, J = 1.6 Hz, 2H), |
| | 8.43 (d, J = 8.0 Hz, 2H), 8.00 (s, 1H), 7.97 (d, J = 8.0 Hz, 2H), 7.70 (d, J = |
| | 10.8 Hz, 1H), 7.08 (t, J = 54.0 Hz, 1H) |
| 68 | ¹H NMR (400 MHz, DMSO-d₆) δ 13.47 (s, 1H), 8.42-8.40 (m, 3H), 7.93 (d, J = |
| | 8.4 Hz, 2H), 7.31 (s, 1H), 7.03 (s, 2H), 6.60 (t, J = 53.6 Hz, 1H) |
| 69 | ¹H NMR (400 MHz, DMSO-d₆) δ 13.62 (s, 1H), 8.42 (d, J = 8.0 Hz, 2H), |
| | 7.97 (d, J = 8.0 Hz, 2H), 7.79 (s, 1H), 7.24 (s, 1H), 6.41 (s, 1H), 6.02 (s, 2H), |
| | 2.05 (s, 3H) |
| 70 | ¹H NMR (400 MHz, DMSO-d₆) δ 13.67 (s, 1H), 8.43 (d, J = 8.0 Hz, 2H), |
| | 8.02 (s, 1H), 7.97 (d, J = 8.0 Hz, 2H), 7.30 (d, J = 5.2 Hz, 1H), 6.85 (s, 1H), |
| | 3.72 (t, J = 4.8 Hz, 4H), 3.51 (t, J = 4.8 Hz, 4H), 2.14 (s, 3H) |
| 71 | ¹H NMR (400 MHz, DMSO-d₆) δ 13.64 (s, 1H), 12.86 (s, 1H), 8.43 (d, J = |
| | 8.0 Hz, 2H), 7.98 (d, J = 8.0 Hz, 2H), 7.52 (s, 1H), 7.47 (s, 1H), 1.68-1.61 (m, |
| | 1H), 0.82-0.77 (m, 2H), 0.53-0.49 (m, 2H) |
| 72 | ¹H NMR (400 MHz, DMSO-d₆) δ 13.68 (s, 1H), 8.44 (d, J = 8.0 Hz, 2H), |
| | 8.16 (s, 1H), 7.99 (d, J = 8.0 Hz, 2H), 7.55 (d, J = 1.2 Hz, 1H), 7.47 (d, J = |
| | 5.2 Hz, 1H), 7.11 (d, J = 1.2 Hz, 1H), 4.23 (p, J = 6.4 Hz, 1H), 1.35 (d, J = |
| | 6.4 Hz, 6H) |
| 73 | ¹H NMR (400 MHz, DMSO-d₆) δ 13.75 (s, 1H), 8.43 (d, J = 8.4 Hz, 2H), |
| | 7.98 (d, J = 8.4 Hz, 2H), 7.34 (s, 1H), 5.57 (s, 1H), 4.74 (s, 2H), 4.11 (p, J = |
| | 6.4 Hz, 1H), 1.28 (d, J = 6.4 Hz, 6H) |
| 74 | ¹H NMR (400 MHz, DMSO-d₆) δ 13.77 (s, 1H), 8.60 (s, 1H), 8.43 (d, J = 8.4 |
| | Hz, 2H), 7.98 (d, J = 8.4 Hz, 2H), 7.41 (s, 1H), 6.74 (t, J = 53.2 Hz, 1H), 3.82 |
| | (t, J = 4.8 Hz, 4H), 3.72 (t, J = 4.8 Hz, 4H) |
| 75 | ¹H NMR (400 MHz, DMSO-d₆) δ 13.83 (s, 1H), 8.44 (d, J = 8.4 Hz, 2H), |
| | 7.98 (d, J = 8.4 Hz, 2H), 7.57 (d, J = 5.2 Hz, 1H), 7.53 (d, J = 2.0 Hz, 1H), |
| | 6.49 (d, J = 2.0 Hz, 1H), 3.65-3.60 (m, 1H), 1.00-0.92 (m, 2H), 0.91-0.83 (m, |
| | 2H) |
| 76 | ¹H NMR (400 MHz, Methanol-d₄) δ 8.28 (d, J = 8.0 Hz, 2H), 8.20 (d, J = 1.2 |
| | Hz, 1H), 7.87 (d, J = 8.0 Hz, 2H), 7.48 (t, J = 51.6 Hz, 1H), 7.40 (d, J = 5.2 |
| | Hz, 1H), 2.38 (s, 3H) |
| 77 | ¹H NMR (400 MHz, DMSO-d₆) δ 13.65 (s, 1H), 8.43 (d, J = 8.4 Hz, 2H), |
| | 7.99-7.97 (m, 3H), 7.32 (s, 1H), 6.93-6.61 (m, 2H), 6.53 (s, 2H) |
| 78 | ¹H NMR (400 MHz, DMSO-d₆) δ 13.66 (s, 1H), 8.43 (d, J = 8.0 Hz, 2H), |
| | 8.09-7.76 (m, 4H), 7.42 (d, J = 5.2 Hz, 1H), 2.47 (s, 3H) |
| 79 | ¹H NMR (400 MHz, DMSO-d₆) δ 13.68 (s, 1H), 8.43 (s, 1H), 8.41-8.36 (m, |
| | 2H), 8.02 (d, J = 8.4 Hz, 2H), 7.51 (s, 2H), 7.38 (s, 1H), 7.32 (s, 2H), 6.67 (t, |
| | J = 53.2 Hz, 1H) |
| 80 | ¹H NMR (400 MHz, DMSO-d₆) δ 13.72 (s, 1H), 8.56 (s, 1H), 8.53 (d, J = 7.6 |
| | Hz, 1H), 8.43 (s, 1H), 7.92 (d, J = 7.6 Hz, 1H), 7.84 (t, J = 7.6 Hz, 1H), 7.40 |
| | (d, J = 5.4 Hz, 1H), 7.31 (s, 2H), 6.67 (t, J = 53.2 Hz, 1H) |
| 81 | ¹H NMR (400 MHz, DMSO-d₆) δ 13.72 (s, 1H), 8.65-8.52 (m, 2H), 8.43 (s, |
| | 1H), 7.78 (dd, J = 10.4, 8.4 Hz, 1H), 7.41 (d, J = 5.2 Hz, 1H), 7.32 (s, 2H), |
| | 6.67 (t, J = 53.2 Hz, 1H) |
| 82 | ¹H NMR (400 MHz, DMSO-d₆) δ 13.40 (s, 1H), 8.11 (s, 1H), 7.90-7.81 (m, |
| | 2H), 7.37 (dd, J = 12.4, 8.4 Hz, 1H), 7.30 (s, 1H), 6.74 (s, 2H), 3.81-3.79 (m, |
| | 4H), 3.14-3.12 (m, 4H), 2.17 (d, J = 1.2 Hz, 3H) |
| 83 | ¹H NMR (400 MHz, DMSO-d₆) δ 13.05 (s, 1H), 8.09 (s, 1H), 7.74 (s, 1H), |
| | 7.63 (d, J = 7.7 Hz, 1H), 7.40 (t, J = 8.0 Hz, 1H), 7.19 (d, J = 5.2 Hz, 1H), |
| | 7.09 (dd, J = 8.0, 2.4 Hz, 1H), 6.43 (s, 2H), 3.81-3.78 (m, 4H), 3.25-3.23 (m, |
| | 4H), 2.17 (d, J = 1.2 Hz, 3H) |
| 84 | ¹H NMR (400 MHz, DMSO-d₆) δ 13.41 (s, 1H), 8.43 (s, 1H), 7.90-7.83 (m, |
| | 2H), 7.41-7.30 (m, 4H), 6.66 (t, J = 53.2 Hz, 1H), 3.66-3.63 (m, 4H), 3.14 (t, |
| | J = 5.2 Hz, 2H), 3.08 (t, J = 5.2 Hz, 2H), 2.07 (s, 3H) |
| 85 | ¹H NMR (400 MHz, DMSO-d₆) δ 13.36 (s, 1H), 8.43 (s, 1H), 7.78 (s, 1H), |
| | 7.66 (d, J = 8.0 Hz, 1H), 7.43 (t, J = 8.0 Hz, 1H), 7.30 (m, 3H), 7.15 (dd, J = |
| | 8.0, 2.4 Hz, 1H), 6.67 (t, J = 53.2 Hz, 1H), 3.68-3.59 (m, 4H), 3.30 (t, J = 5.2 |
| | Hz, 2H), 3.24 (t, J = 5.2 Hz, 2H), 2.07 (s, 3H) |
| 86 | ¹H NMR (400 MHz, DMSO-d₆) δ 13.41 (s, 1H), 8.43 (s, 1H), 7.88-7.84 (m, |
| | 2H), 7.40-7.31 (m, 4H), 6.67 (t, J = 53.2 Hz, 2H), 3.80 (t, J = 4.8 Hz, 4H), |
| | 3.13 (t, J = 4.8 Hz, 4H) |
| 87 | ¹H NMR (400 MHz, DMSO-d₆) δ 13.37 (s, 1H), 8.43 (s, 1H), 7.77 (s, 1H), |
| | 7.66 (d, J = 7.6 Hz, 1H), 7.43 (t, J = 8.0 Hz, 1H), 7.31 (s, 3H), 7.14 (d, J = 8.0 |
| | Hz, 1H), 6.67 (t, J = 53.2 Hz, 1H), 3.80 (t, J = 4.8 Hz, 4H), 3.24 (t, J = 4.8 |
| | Hz, 4H) |
| 88 | ¹H NMR (400 MHz, DMSO-d₆) δ 13.48 (s, 1H), 8.44-8.40 (m, 3H), 7.93 (d, J = |
| | 8.4 Hz, 2H), 7.52 (s, 1H), 7.36 (s, 1H), 6.61 (t, J = 53.2 Hz, 1H), 3.56-3.54 |
| | (m, 4H), 3.30 (s, 3H) |
| 89 | ¹H NMR (400 MHz, DMSO-d₆) δ 13.52 (s, 1H), 8.43 (s, 1H), 8.19 (s, 1H), |
| | 8.10 (d, J = 8.4 Hz, 1H), 7.66 (d, J = 8.4 Hz, 1H), 7.32 (s, 3H), 6.67 (t, J = |
| | 53.2 Hz, 1H) |
| 90 | ¹H NMR (400 MHz, DMSO-d₆) δ 13.69 (s, 1H), 8.43 (s, 1H), 8.29 (dd, J = |
| | 2.0, 2.0 Hz, 1H), 8.17 (d, J = 8.8 Hz, 1H), 7.83 (t, J = 8.4 Hz, 1H), 7.40 (s, |
| | 1H), 7.32 (s, 2H), 6.67 (d, J = 53.2 Hz, 1H) |
| 91 | ¹H NMR (400 MHz, DMSO-d₆) δ 13.75 (s, 1H), 8.71 (s, 1H), 8.52 (s, 1H), |
| | 8.43 (d, J = 8.0 Hz, 2H), 7.98 (d, J = 8.0 Hz, 2H), 7.39 (s, 1H), 6.71 (t, J = |
| | 53.2 Hz, 1H), 5.05-4.96 (m, 1H), 4.81 (t, J = 6.4 Hz, 2H), 4.58 (t, J = 6.4 Hz, |
| | 2H) |
| 92 | ¹H NMR (400 MHz, DMSO-d₆) δ 13.33 (s, 1H), 8.39 (d, J = 8 Hz, 2H), 7.92 |
| | (d, J = 8.4 Hz, 2H), 7.71 (s, 1H), 7.26 (s, 1H), 5.98 (s, 2H), 2.77 (s, 6H) |
| 93 | ¹H NMR (400 MHz, DMSO-d₆) δ 13.42 (s, 1H), 8.41 (d, J = 8.2 Hz, 2H), |
| | 8.19 (s, 1H), 7.92 (d, J = 8.2 Hz, 2H), 7.28 (s, 1H), 5.46 (s, 1H), 4.59-4.58 (m, |
| | 1H), 4.31-4.26 (m, 2H), 3.86-3.82 (m, 2H), 2.22 (d, J = 1.2 Hz, 3H) |
| 94 | ¹H NMR (400 MHz, DMSO-d6) δ 13.59 (s, 1H), 8.43 (s, 1H), 8.36 (d, J = 7.2 |
| | Hz, 1H), 8.31 (ddd, J = 8.8, 4.8, 2.0 Hz, 1H), 7.78 (dd, J = 10.0, 8.8 Hz, 1H), |
| | 7.39 (s, 1H), 7.31 (s, 2H), 6.67 (t, J = 53.2 Hz, 1H) |
| 95 | ¹H NMR (400 MHz, DMSO-d₆) δ 13.84 (s, 1H), 8.68 (s, 1H), 8.45 (d, J = 8.0 |
| | Hz, 2H), 7.99 (d, J = 8.0 Hz, 2H), 7.62-7.59 (m, 3H) |
| 96 | ¹H NMR (400 MHz, DMSO-d6) δ 13.70 (s, 1H), 12.61 (s, 1H), 8.43 (d, J = |
| | 8.0 Hz, 2H), 8.27 (s, 1H), 7.98 (d, J = 8.0 Hz, 2H), 7.35 (d, J = 5.2 Hz, 1H), |
| | 4.28 (t, J = 8.8 Hz, 2H), 4.13 (dd, J = 8.8, 5.6 Hz, 2H), 3.59-3.53 (m, 1H), |
| | 2.24 (d, J = 1.3 Hz, 3H) |
| 97 | ¹H NMR (400 MHz, DMSO-d₆) δ 13.79 (s, 1H), 8.69 (s, 1H), 8.45 (d, J = 8.0 |
| | Hz, 2H), 7.99 (d, J = 8.0 Hz, 2H), 7.48 (d, J = 5.2 Hz, 1H), 3.74 (s, 2H), 3.60 |
| | (t, J = 4.4 Hz, 4H), 2.57 (t, J = 4.4 Hz, 4H), 2.41 (s, 3H) |
| 98 | ¹H NMR (400 MHz, DMSO-d₆) δ 13.54 (s, 1H), 8.65 (s, 1H), 8.43 (d, J = 8.4 |
| | Hz, 2H), 7.94 (d, J = 8.4 Hz, 2H), 7.41 (s, 1H), 3.82 (s, 2H), 3.47-3.46 (m, |
| | 4H), 2.61 (m, 4H), 2.40 (d, J = 1.6 Hz, 3H), 1.98 (s, 3H) |
| 99 | ¹H NMR (400 MHz, DMSO-d₆) δ 13.73 (s, 2H), 8.42-8.37 (m, 3H), 7.95 (d, J = |
| | 8.0 Hz, 2H), 7.44 (s, 1H), 7.04 (s, 2H) |
| 100 | ¹H NMR (400 MHz, DMSO-d₆) δ 13.16 (s, 1H), 8.11 (s, 1H), 8.04 (s, 1H), |
| | 7.99 (d, J = 8.4 Hz, 1H), 7.51 (d, J = 8.4 Hz, 1H), 7.24 (s, 1H), 6.45 (s, 2H), |
| | 3.46 (s, 3H), 2.19 (d, J = 1.2 Hz, 3H) |
| 101 | ¹H NMR (400 MHz, DMSO-d₆) δ 13.85 (s, 1H), 9.16 (s, 1H), 8.57 (d, J = 8.4 |
| | Hz, 1H), 8.45 (dd, J = 8.4, 2.0 Hz, 1H), 8.13 (s, 1H), 7.27 (s, 1H), 6.80 (s, |
| | 2H), 2.17 (d, J = 1.2 Hz, 3H) |
| 102 | ¹H NMR (400 MHz, DMSO-d₆) δ 8.45 (d, J = 8.0 Hz, 2H), 8.24 (d, J = 1.2 |
| | Hz, 1H), 8.13 (d, J = 10.0 Hz, 1H), 7.97 (d, J = 8.0 Hz, 2H), 6.83 (s, 2H), |
| | 2.25 (d, J = 1.6 Hz, 3H) |
| 103 | ¹H NMR (400 MHz, DMSO-d₆) δ 13.71 (s, 1H), 8.44 (d, J = 8.0 Hz, 2H), |
| | 7.98 (d, J = 8.4 Hz, 2H), 7.40 (s, 1H), 6.71 (d, J = 1.2 Hz, 1H), 6.45 (s, 2H), |
| | 2.06 (d, J = 1.2 Hz, 3H) |
| 104 | ¹H NMR (400 MHz, DMSO-d₆) δ 12.96 (s, 1H), 8.20 (s, 1H), 7.85-7.80 (m, |
| | 1H), 7.71-7.65 (m, 1H), 7.48 (dd, J = 7.6, 2.0 Hz, 1H), 7.25 (s, 1H), 5.47 (s, |
| | 1H), 4.63-4.57 (m, 1H), 4.31-4.27 (m, 2H), 3.87-3.83 (m, 2H), 2.84 (d, J = |
| | 2.0 Hz, 6H), 2.23 (d, J = 1.2 Hz, 3H) |
| 105 | ¹H NMR (400 MHz, DMSO-d₆) δ 13.22 (s, 1H), 12.74 (s, 1H), 8.26 (s, 1H), |
| | 7.87-7.82 (m, 1H), 7.76-7.71 (m, 1H), 7.50 (dd, J = 7.6, 1.2 Hz, 1H), 7.30 (d, |
| | J = 5.2 Hz, 1H), 4.26 (t, J = 8.8 Hz, 2H), 4.12 (dd, J = 8.4, 5.6 Hz, 2H), 3.56- |
| | 3.48 (m, 1H), 2.82 (s, 6H), 2.24 (s, 3H) |

### Example 2

This example relates to the relationship between the structure and activity of (hetero)aryl imidazole compounds containing an aromatic substituent. Specifically, the biochemical activity of the (hetero)aryl imidazole compounds containing an aromatic substituent described in Example 1 as sEH enzyme inhibitors was evaluated by in vitro activity test experiments.

The experimental procedures for evaluating in vitro biochemical activity were described below.

The substrate used in the evaluation experiment was epoxyeicosatrienoic acid (EET). The solutions used in the evaluation experiments were Bis/Tris HCl 25 mM pH 7.0, Bis/Tris HCl 25 mM pH 7.0 containing 0.1 mg/mL BSA was used as buffer A, and Bis/Tris HCl 25 mM pH 7.0 containing 36.3 mM ZnSO₄ was used as buffer B.

The specific steps of the evaluation experiment were as follows. 79 μL of buffer A was added to each well in a black 96-well plate and then 1 μL solution of the aromatic substituent-containing (hetero)aryl imidazole compound was added. Then, 10 μL sEH (1 μM) buffer A solution was added to the wells. After pre-incubation at room temperature for 90 min, 10 μL EET (10 μM) buffer A solution was added to trigger the reaction. After the mixture was allowed to react outdoors for 60 min, 15 μL buffer B and 180 μL acetonitrile were added to quench the reaction, followed by high-speed centrifugation and filtration for later use.

LC-MS was used to detect the concentration of EET in the samples. The final IC₅₀ was calculated based on three independent experiments using Graphpad Prism software.

The inhibitory activity IC₅₀ values of some compounds against soluble epoxide hydrolase obtained through in vitro biochemical activity evaluation experiments are shown in Table 3.

TABLE 3
Activity test results of some compounds

| | | Compound | | Compound | |
|---|---|---|---|---|---|
| No. | IC₅₀ (nM) | No. | IC₅₀ (nM) | No. | IC₅₀ (nM) |
| 1 | 0.11 | 21 | 0.051 | 41 | 0.23 |
| 2 | 11 | 22 | 0.10 | 42 | 0.14 |
| 3 | 7.6 | 23 | 0.29 | 44 | 0.096 |
| 4 | 0.99 | 24 | 0.20 | 45 | 0.033 |
| 5 | 0.074 | 25 | 0.045 | 46 | 0.042 |
| 6 | 0.085 | 26 | 0.081 | 47 | 0.036 |
| 7 | 0.11 | 27 | 0.056 | 51 | 0.016 |
| 8 | 0.065 | 28 | 0.046 | 55 | 0.10 |
| 9 | 0.11 | 29 | 0.083 | 57 | 0.036 |
| 10 | 0.095 | 30 | 0.10 | 60 | 0.057 |
| 11 | 0.14 | 31 | 0.085 | 68 | 0.089 |
| 12 | 0.058 | 32 | 0.13 | 72 | 0.77 |
| 13 | 0.40 | 33 | 0.15 | 74 | 0.17 |
| 14 | 0.94 | 34 | 0.058 | 75 | 0.081 |
| 15 | 0.62 | 35 | 0.045 | 76 | 0.081 |
| 16 | 0.14 | 36 | 0.54 | 77 | 0.047 |
| 17 | 4.8 | 37 | 0.38 | 80 | 0.45 |
| 18 | 436 | 38 | 0.045 | 81 | 0.15 |
| 19 | 0.98 | 39 | 0.088 | | |
| 20 | 2.21 | 40 | 0.063 | | |

### Example 3

This example relates to the relationship between the structure and activity of (hetero)aryl imidazole compounds containing an aromatic substituent. Specifically, the biochemical activity of the (hetero)aryl imidazole compounds containing an aromatic substituent described herein as sEH enzyme inhibitors was evaluated by PHOME assay activity test experiment. The synthesis of the substrates and the test methods required for this test were all based on known literature reports (Analytical Biochemistry 2005, 343, 66-75).

The sEH enzyme activity inhibition experiment was performed in a low binding 96-well plate. The reaction system was 100 μL 25 mM Bis-Tris, pH 7.0, 0.1 mg/mL BSA buffer. The experimental steps of PHOME assay activity test were as follows.

First, a sEH enzyme solution with a final concentration of 500 μM was mixed with compounds of different concentrations (test compound concentration range 20 nM-0.082 nM), and then pre-incubated on a shaker at room temperature for 10 min. The reaction was triggered by adding Phome fluorescent substrate at a final concentration of 5 μM and immediately placed in a Perkin Elmer microplate reader for dynamic monitoring for 1 hour at an excitation wavelength of 330 nm and an emission wavelength of 465 nm, with fluorescence readings at intervals of 1 minute. Graphpad Prism 9.0 software was used to calculate the slope of the fluorescence value increase in each well within 20 minutes, and the slope was used to calibrate the enzyme activity to calculate the inhibition rate of the compound on sEH enzyme activity under different concentration conditions. Finally, Graphpad Prism 9 was used to plot and calculate IC₅₀ values.

The inhibitory activity IC₅₀ values of some compounds against soluble epoxide hydrolase obtained by PHOME assay activity evaluation experiment are shown in Table 4.

TABLE 4
Activity test results of some compounds

| Compound No. | IC₅₀ (nM) |
|---|---|
| 80 | 0.65 |
| 88 | 0.65 |
| 89 | 0.35 |
| 90 | 0.48 |
| 91 | 0.61 |
| 92 | <0.082 |
| 93 | <0.082 |
| 94 | 2.54 |
| 95 | <0.082 |
| 96 | 0.14 |
| 97 | 0.092 |
| 98 | 0.11 |
| 99 | 41.6 |
| 100 | 10.1 |
| 101 | 0.35 |
| 102 | 1.45 |
| 103 | 0.36 |
| 104 | >20 |
| 105 | >20 |

### Example 4

This example relates to the evaluation of the pharmacokinetic properties of (hetero)aryl imidazole compounds containing an aromatic substituent. Specifically, this experiment evaluated the in vivo pharmacokinetic properties of some of the (hetero)aryl imidazole compounds with an aromatic substituent synthesized in Example 1 in mice under intravenous (IV) and oral (PO) administration.

The animals used in this experiment were male ICR mice, which were obtained from Beijing Weitonglihua Experimental Animal Technology Co., Ltd. IV/PO administration was performed with a single drug or a combination of drugs (Cassette, no more than five drugs), the solvent was 5% DMSO+10% Solutol+85% Saline, the compound concentration was 0.2 mg/mL, and the IV/PO administration volume was 5/10 mL/kg, respectively. The animals in the oral administration group were fasted overnight before administration and fed 4 hours after administration. After administration, blood was collected from the cheek at the set time of the experiment. About 50 μL was collected for each sample and anticoagulated with sodium heparin. After collection, the blood was placed on wet ice and centrifuged within 1 hour to separate the plasma (6000 rpm, 3 minutes, 2-8° C.). Plasma samples were stored at −80° C. before analysis. The plasma samples were diluted and analyzed by LC-MS/MS to determine the blood drug concentration data in the samples at different time points, and Phoenix WinNonlin 8.2.0 was used to calculate the pharmacokinetic parameters. The pharmacokinetic test results of some compounds are shown in Table 5.

TABLE 5
Pharmacokinetic test results of some compounds

| | intravenous injection | | | Oral administration | | | bio- |
|---|---|---|---|---|---|---|---|
| Com- | administration (IV) | | | by gavage (PO) | | | avail- |
| pound | dosage | T_{1/2} | C1 | dosage | T_{1/2} | AUC | ability |
| No. | (mpk) | (h) | (mL/kg/min) | (mpk) | (h) | (h * ng/ml) | (%) |
| 8 | 1 | 5.9 | 1.3 | 2 | 6.4 | 19595 | 76 |
| 61 | 1 | 8.5 | 1.0 | 2 | 10.2 | 26092 | 83 |

From the results of the above-mentioned examples, it can be seen that the (hetero)aryl imidazole compounds containing an aromatic substituent in the present invention have strong inhibitory activity against soluble epoxide hydrolase, and the activities of some compounds are even lower than 100 μM. From the results in Table 3 and Table 4, it can be seen that the aromatic substituent of Ar1 plays a crucial role in the high inhibitory activity of the compounds of the present invention. At the same time, pharmacokinetic tests show that the compounds of the present invention are metabolically stable, have high bioavailability, and have very good pharmacokinetic properties. In general, the (hetero)aryl imidazole compounds containing an aromatic substituent in the present invention are a class of highly active soluble epoxide hydrolase inhibitors with excellent properties.

The above description of the examples is intended to facilitate those skilled in the art to understand and apply the present application. It will be apparent to those skilled in the art that they can easily make various modifications to these examples and apply the general principles described herein to other examples without incurring creative effort. Therefore, the present application is not limited to the examples herein, and improvements and modifications made by those skilled in the art based on the contents disclosed in the present application without departing from the scope and spirit of the present application are within the scope of the present application.

1. An aromatic substituent-containing heteroaryl- or phenyl-imidazole compound, a stereoisomer, a hydrate, a solvate, a pharmaceutically acceptable salt or a prodrug thereof, wherein the compound has a structure represented by the following general formula IA or general formula IB: 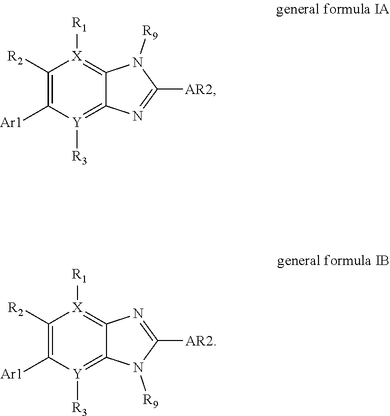
wherein, X and Y each independently represent C or N;
R₁, R₂ and R₃ are each independently selected from a group consisting of hydrogen, deuterium, tritium, halogen, substituted or unsubstituted straight-chain alkyl, substituted or unsubstituted branched-chain alkyl, substituted or unsubstituted heteroalkyl, substituted or unsubstituted cycloalkyl, substituted or unsubstituted heterocycloalkyl, cyano, trifluoromethyl, alkoxy, cycloalkyloxy, aryloxy, substituted or unsubstituted amino, substituted or unsubstituted thiol, substituted or unsubstituted sulfonyl, substituted or unsubstituted alkenyl, substituted or unsubstituted alkynyl, substituted or unsubstituted heteroalkenyl, substituted or unsubstituted heteroalkynyl, substituted or unsubstituted aryl, and substituted or unsubstituted heteroaryl, or isotopic substituents thereof;
R₉ is selected from a group consisting of hydrogen, deuterium, tritium, halogen, substituted or unsubstituted straight chain alkyl, substituted or unsubstituted branched chain alkyl, substituted or unsubstituted heteroalkyl, substituted or unsubstituted cycloalkyl, substituted or unsubstituted heterocycloalkyl, cyano, trifluoromethyl, alkoxy, cycloalkyloxy, aryloxy, substituted or unsubstituted amino, substituted or unsubstituted thiol, substituted or unsubstituted sulfonyl, substituted or unsubstituted alkenyl, substituted or unsubstituted alkynyl, substituted or unsubstituted heteroalkenyl, substituted or unsubstituted heteroalkynyl, substituted or unsubstituted aryl, and substituted or unsubstituted heteroaryl, or isotopic substituents thereof, or a prodrug group of a parent structure;
Ar1 and Ar2 are each independently selected from a group consisting of substituted or unsubstituted aryl, substituted or unsubstituted heteroaryl;
wherein, at least one unsaturated carbon atom in the heteroaryl or phenyl ring of the heteroaryl- or phenyl-imidazole compound is directly connected to an unsaturated carbon atom of Ar1, and a carbon atom adjacent to two N atoms in the imidazole ring of the heteroaryl- or phenyl-imidazole compound is directly connected to an unsaturated carbon atom of Ar2.

2. The compound according to claim 1, the stereoisomer, hydrate, solvate, pharmaceutically acceptable salt or prodrug thereof, wherein X and Y are C or N, wherein when X and Y are N, R₁ and R₃ are absent;
R₁, R₂ and R₃ are each independently selected from a group consisting of hydrogen, deuterium, tritium, halogen, alkyl, haloalkyl, cycloalkyl, alkoxy, heterocycloalkyl, cyano, aryl, heteroaryl;
R₉ is hydrogen, deuterium, tritium, alkyl, haloalkyl, alkoxy, haloalkoxy, or sulfuryl;
Ar1 is selected from a group consisting of substituted or unsubstituted aryl, substituted or unsubstituted heteroaryl, wherein the substituted refers to substitution by one or more substituents selected from a group consisting of cyano, carboxyl, ester group, hydroxyl, alkyl, haloalkyl, alkoxy, cycloalkyloxy, heterocyclyloxy, halogen, heterocyclyl, alkylene-heterocyclyl, cycloalkyl, —COalkyl, —S(O)₂-alkyl, ═O, aryl, heteroaryl, haloalkoxy, —NRaRb, -alkyleneNRaRb, —SO₂NRaRb, —CONRaRb, and each of the above substituents may be further substituted by one or more substituents selected from a group consisting of halogen, hydroxyl, amino, alkyl, haloalkyl, cycloalkyl, halocycloalkyl, heterocyclyl, halogenated heterocyclyl, alkoxy, haloalkoxy, cycloalkyloxy, halocycloalkyloxy, heterocyclyloxy, and halogenated heterocyclyloxy;
Ar2 is selected from a group consisting of substituted or unsubstituted aryl, substituted or unsubstituted heteroaryl, wherein the substituted refers to substitution by one or more substituents selected from a group consisting of halogen, heterocyclyl, haloalkyl, ═O, =—SO₂alkyl, =—SO₂NRaRb, —CONRaRb, alkoxy, alkyl, haloalkoxy, alkyl-substituted thiol, haloalkyl-substituted thiol, cyano, ester group, aryl, heteroaryl, NRaRb, and each of the above substituents may be further substituted by one or more substituents selected from a group consisting of halogen, hydroxy, alkyl, haloalkyl, cycloalkyl, halocycloalkyl, heterocyclyl, haloheterocyclyl, alkoxy, haloalkoxy, cycloalkyloxy, halocycloalkyloxy, heterocyclyloxy, and haloheterocyclyloxy;
each Ra and each Rb, at each occurrence, are independently selected from: H, alkyl, heteroalkyl, heterocyclyl, acyl, sulfuryl, or sulfonyl; or at each occurrence, Ra and Rb optionally form a 3-8 membered heterocycle with the connected N, the above alkyl, 4-8 membered heterocycle may be optionally substituted with one or more substituents selected from a group consisting of: OH, COOH, CONH₂, COalkyl, alkoxy, alkyl, haloalkoxy, haloalkyl, aryl, heteroaryl, amino, monoalkylamino, dialkylamino;
preferably, X and Y are C;
R₁, R₂ and R₃ are each independently selected from a group consisting of hydrogen, deuterium, tritium, halogen, alkyl, and haloalkyl;
R₉ is hydrogen;
Ar1 is a substituted or unsubstituted heteroaryl, wherein the substituted refers to substitution by one or more substituents selected from a group consisting of NRaRb, -alkylene NRaRb, cyano, carboxyl, ester group, hydroxyl, alkyl, haloalkyl, alkoxy, haloalkoxy, halogen, heterocyclyl, alkylene-heterocyclyl, cycloalkyl, —COalkyl, —SO₂alkyl, ═O, and each of the above substituents may be further substituted by one or more substituents selected from a group consisting of halogen, hydroxyl, amino, alkyl, haloalkyl, alkoxy, haloalkoxy, cycloalkyloxy, halocycloalkyloxy, heterocyclyloxy, and haloheterocyclyloxy;
Ar2 is substituted or unsubstituted aryl, substituted or unsubstituted heteroaryl, wherein the substituted refers to substitution by one or more substituents selected from a group consisting of halogen, heterocyclyl, ═O, haloalkyl, —SO₂alkyl, —SO₂NRaRb, —CONRaRb, alkoxy, NRaRb, alkyl, cyano, ester group, thiol substituted by haloalkyl, and each of the above substituents may be further substituted by one or more substituents selected from a group consisting of halogen, hydroxyl, alkyl, haloalkyl, alkoxy, haloalkoxy, cycloalkyloxy, halocycloalkyloxy, heterocyclyloxy, and halogenated heterocyclyloxy;
each Ra and each Rb, at each occurrence, are independently selected from: H, alkyl, or heterocyclyl; or at each occurrence, Ra and Rb, optionally, with the connected N, form a 3-8 membered heterocycle, wherein the alkyl and heterocycle may be optionally substituted with one or more substituents selected from a group consisting of: OH, COOH, CONH₂, COalkyl, alkoxy, alkyl, haloalkoxy, haloalkyl, aryl, heteroaryl, amino, monoalkylamino, and dialkylamino;
more preferably, X and Y are C;
R₁, R₂ and R₃ are each independently selected from a group consisting of hydrogen, deuterium, tritium, F, Cl, Br, and I;
R₉ is hydrogen;
Ar1 is a substituted or unsubstituted 5-8 membered heteroaryl, wherein the substituted refers to substitution by one or more substituents selected from a group consisting of NRaRb, —C₁₋₄ alkylene NRaRb, cyano, carboxyl, C₁₋₆ alkyl, halogenated C₁₋₆ alkyl, C₁₋₆ alkoxy, halogen, 3-8 membered heterocyclyl, —C₁₋₄ alkylene-3-8 membered heterocyclyl, C₃₋₆ cycloalkyl, —CO—C₁₋₆ alkyl, —COOC₁₋₆ alkyl, —SO₂—C₁₋₆ alkyl, ═O, and hydroxyl;
Ar2 is substituted or unsubstituted phenyl, substituted or unsubstituted 5-9 membered heteroaryl, wherein the substituted refers to substitution by one or more substituents selected from a group consisting of halogen, 3-8 membered heterocyclyl, ═O, halogenated C₁₋₆ alkyl, —SO₂C₁₋₆ alkyl, C₁₋₆ alkyl, —SO₂NRaRb, —CONRaRb, —NRaRb, C₁₋₆ alkoxy, and C₁₋₆ halogenated alkoxy;
each Ra and each Rb, at each occurrence, are independently selected from: H, C₁₋₆ alkyl, or 3-6 membered heterocyclyl; or Ra and Rb, at each occurrence, optionally form a 3-6 membered heterocycle with the connected N, and the above alkyl and heterocyclyl may be optionally substituted by one or more substituents selected from a group consisting of: OH, COOH, CONH₂, COC₁₋₆ alkyl, and C₁₋₆ alkoxy.

3. The compound according to claim 1, the stereoisomer, hydrate, solvate, pharmaceutically acceptable salt or prodrug thereof, wherein the compound has a structure represented by the following general formula IIA or IIB: 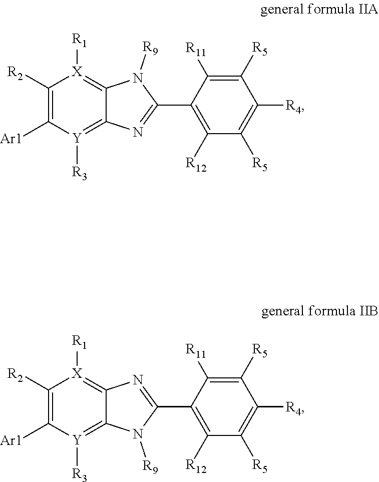
wherein, R₄, R₅, R₆, R₁₁ and R₁₂ are each independently selected from a group consisting of: hydrogen, deuterium, tritium, halogen, substituted or unsubstituted straight-chain alkyl, substituted or unsubstituted branched-chain alkyl, substituted or unsubstituted heteroalkyl, substituted or unsubstituted cycloalkyl, substituted or unsubstituted heterocycloalkyl, cyano, trifluoromethyl, alkoxy, cycloalkyloxy, aryloxy, substituted or unsubstituted amino, substituted or unsubstituted thiol, substituted or unsubstituted sulfonyl, substituted or unsubstituted alkenyl, substituted or unsubstituted alkynyl, substituted or unsubstituted heteroalkenyl, substituted or unsubstituted heteroalkynyl, substituted or unsubstituted aryl, and substituted or unsubstituted heteroaryl, or isotopic substituents thereof.

4. The compound according to claim 3, the stereoisomer, hydrate, solvate, pharmaceutically acceptable salt or prodrug thereof, wherein R₄, R₅, R₆, R₁₁ and R₁₂ are each independently selected from a group consisting of: halogen, heterocyclyl, ═O, haloalkyl, haloalkoxy, —SO₂alkyl, —SO₂NRaRb, —CONRaRb, alkoxy, NRaRb, alkyl, cyano, haloalkyl substituted thiol, ester group; adjacent substituents form a substituted or unsubstituted heterocycle with the connected C, and the substituted heterocycle has one or more substituents selected from halogen, alkyl, or ═O;
each Ra and each Rb, at each occurrence, are independently selected from: H, alkyl, or heterocyclyl; or at each occurrence, Ra, Rb, optionally, with the attached N, form a heterocycle, and the alkyl and heterocycle may optionally be substituted with one or more substituents selected from a group consisting of: OH, COOH, CONH₂, COalkyl, alkoxy, alkyl, haloalkoxy, haloalkyl, aryl, heteroaryl, amino, monoalkylamino, and dialkylamino;
preferably, R₄, R₅, R₆, R₁₁ and R₁₂ are each independently selected from a group consisting of: halogen, 3-8 membered heterocyclyl, ═O, halogenated C₁₋₆ alkyl, halogenated C₁₋₆ alkoxy, —SO₂C₁₋₆ alkyl, —SO₂NRaRb, —CONRaRb, C₁₋₆ alkoxy, NRaRb, C₁₋₆ alkyl, cyano, —S-halogenated C₁₋₆ alkyl, —COOC₁₋₆ alkyl; adjacent substituents form a substituted or unsubstituted 3-8 membered heterocycle with the connected C, and the substituted heterocycle has one or more substituents selected from halogen, C₁₋₆ alkyl, or ═O;
each Ra and each Rb, at each occurrence, are independently selected from: H, C₁₋₆ alkyl, or 3-8 membered heterocyclyl; or Ra and Rb, at each occurrence, optionally form a 3-8 membered heterocycle with the connected N, and the alkyl and the heterocycle may optionally be substituted by one or more substituents selected from a group consisting of: OH, COOH, CONH₂, COC₁₋₆ alkyl, C₁₋₆ alkoxy, C₁₋₆ alkyl, halogenated C₁₋₆ alkoxy, halogenated C₁₋₆ alkyl, phenyl, 3-8 membered heteroaryl, amino, mono C₁₋₆ alkylamino, and di C₁₋₆ alkylamino.

5. The compound according to claim 3, the stereoisomer, hydrate, solvate, pharmaceutically acceptable salt or prodrug thereof, wherein R₁ and R₂ are connected to form a five-membered ring, a six-membered ring, a seven-membered ring or an eight-membered ring;
and/or, R₄ and R₅ are connected to form a five-membered ring, a six-membered ring, a seven-membered ring or an eight-membered ring;
and/or, R₄ and R₆ are connected to form a five-membered ring, a six-membered ring, a seven-membered ring or an eight-membered ring.

6. The compound according to claim 1, the stereoisomer, hydrate, solvate, pharmaceutically acceptable salt or prodrug thereof, wherein Ar1 has a structure represented by the following general formula III: 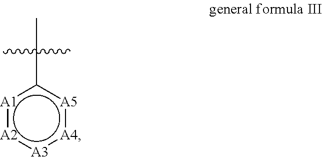
wherein A1, A2, A3, A4 and A5 are each independently selected from a group consisting of CR, N, N—R, S and O, wherein R is each independently selected from a group consisting of hydrogen, deuterium, tritium, halogen, cyano, trifluoromethyl, alkoxy, cycloalkyloxy, aryloxy, substituted or unsubstituted amino, substituted or unsubstituted thiol, substituted or unsubstituted sulfonyl, substituted or unsubstituted straight-chain alkyl, substituted or unsubstituted branched-chain alkyl, substituted or unsubstituted heteroalkyl, substituted or unsubstituted cycloalkyl, substituted or unsubstituted heterocycloalkyl, substituted or unsubstituted alkenyl, substituted or unsubstituted alkynyl, substituted or unsubstituted heteroalkenyl, substituted or unsubstituted heteroalkynyl, substituted or unsubstituted aryl, and substituted or unsubstituted heteroaryl, or isotopic substituents thereof;
preferably, A1, A2, A3, A4, A5 are each independently selected from: CR, N, N—R, S or O, wherein R at each occurrence is independently selected from: hydrogen, deuterium, tritium, halogen, cyano, carboxyl, alkyl, hydroxyl, NRaRb, -alkylene NRaRb, alkoxy, haloalkyl, haloalkoxy, heterocycloalkyl, alkylene heterocycloalkyl, or ═O;
each Ra and each Rb, at each occurrence, are independently selected from: H, alkyl, or heterocyclyl; or at each occurrence, Ra, Rb, optionally, with the connected N, form a 5-8 membered heterocycle, wherein the above alkyl, 5-8 membered heterocycle may be optionally substituted with one or more substituents selected from a group consisting of: OH, COOH, CONH₂, COalkyl, alkoxy, alkyl, haloalkoxy, haloalkyl, aryl, heteroaryl, amino, monoalkylamino, and dialkylamino;
more preferably, A1, A2, A3, A4, A5 are each independently selected from CR or N, wherein R is selected from hydrogen, deuterium, tritium, F, Cl, Br, cyano, carboxyl, hydroxyl, C₁₋₆ alkyl, C₁₋₆ haloalkyl, C₁₋₆ alkoxy, 5-7 membered heterocycloalkyl, C₁₋₆ alkyl substituted 5-7 membered heterocycloalkyl, ═O, —NRaRb, or -alkyleneNRaRb;
Ra and Rb, at each occurrence, are each independently selected from H, C₁₋₆ alkyl or 5-7 membered heterocyclyl; or Ra and Rb, at each occurrence, optionally form a 5-7 membered heterocycle with the connected N, and the above alkyl and heterocycle are optionally substituted by one or more substituents selected from OH, COOH, CONH₂, COC₁₋₆ alkyl, or C₁₋₆ alkyl.

7. The compound according to claim 1, the stereoisomer, hydrate, solvate, pharmaceutically acceptable salt or prodrug thereof, wherein Ar1 has a structure represented by the following general formula IV: 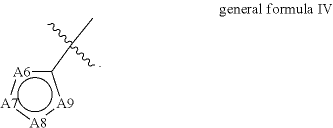
wherein A6, A7, A8 and A9 are each independently selected from a group consisting of CR, N, N—OR, S and O, wherein R is each independently selected from a group consisting of hydrogen, deuterium, tritium, halogen, cyano, trifluoromethyl, alkoxy, cycloalkyloxy, aryloxy, substituted or unsubstituted amino, substituted or unsubstituted thiol, substituted or unsubstituted sulfonyl, substituted or unsubstituted straight-chain alkyl, substituted or unsubstituted branched-chain alkyl, substituted or unsubstituted heteroalkyl, substituted or unsubstituted cycloalkyl, substituted or unsubstituted heterocycloalkyl, substituted or unsubstituted alkenyl, substituted or unsubstituted alkynyl, substituted or unsubstituted heteroalkenyl, substituted or unsubstituted heteroalkynyl, substituted or unsubstituted aryl, and substituted or unsubstituted heteroaryl, or isotopic substituents thereof;
preferably, A6, A7, A8, A9 are each independently selected from CR or N, wherein R is selected from alkyl, haloalkyl, H, cycloalkyl, —COalkyl, —SO₂-alkyl, NH₂, —NHalkyl, or —N(alkyl)₂;
more preferably, A6, A7, A8, A9 are each independently selected from CR or N, wherein R is selected from C₁₋₆ alkyl, H, C₃₋₆ cycloalkyl, —CO—C₁₋₆ alkyl, —SO₂—C₁₋₆ alkyl, NH₂, —NHC₁₋₆ alkyl, —N(C₁₋₆ alkyl)₂ or halogenated C₁₋₆ alkyl.

8. The compound according to claim 1, the stereoisomer, hydrate, solvate, pharmaceutically acceptable salt or prodrug thereof, wherein the compound is selected from a group consisting of:

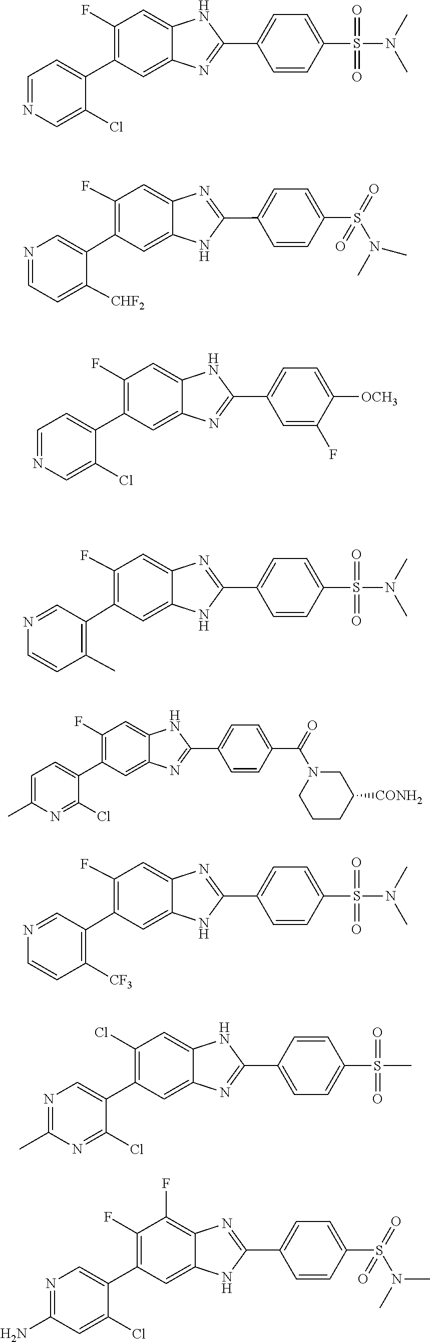

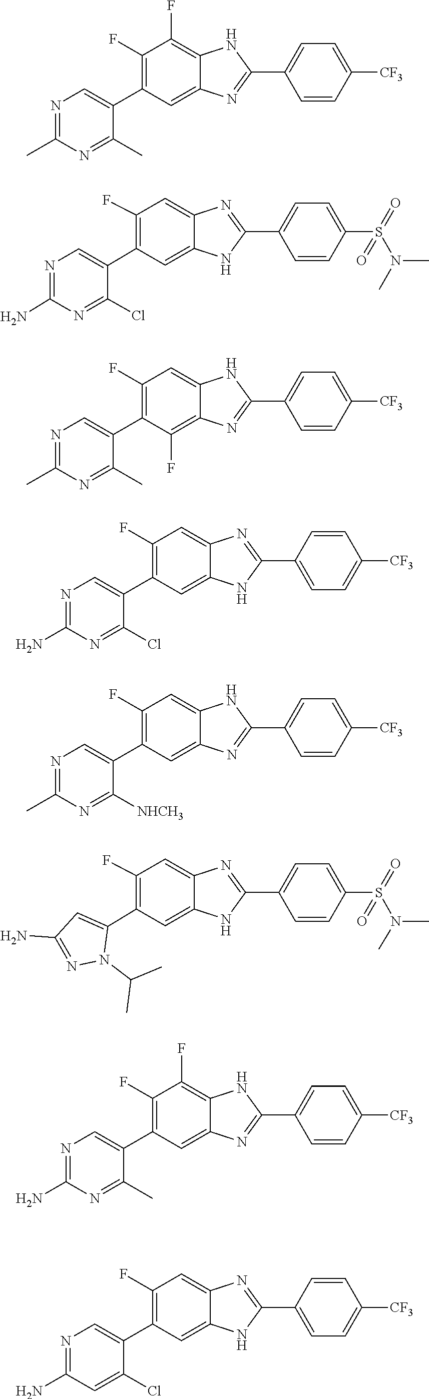

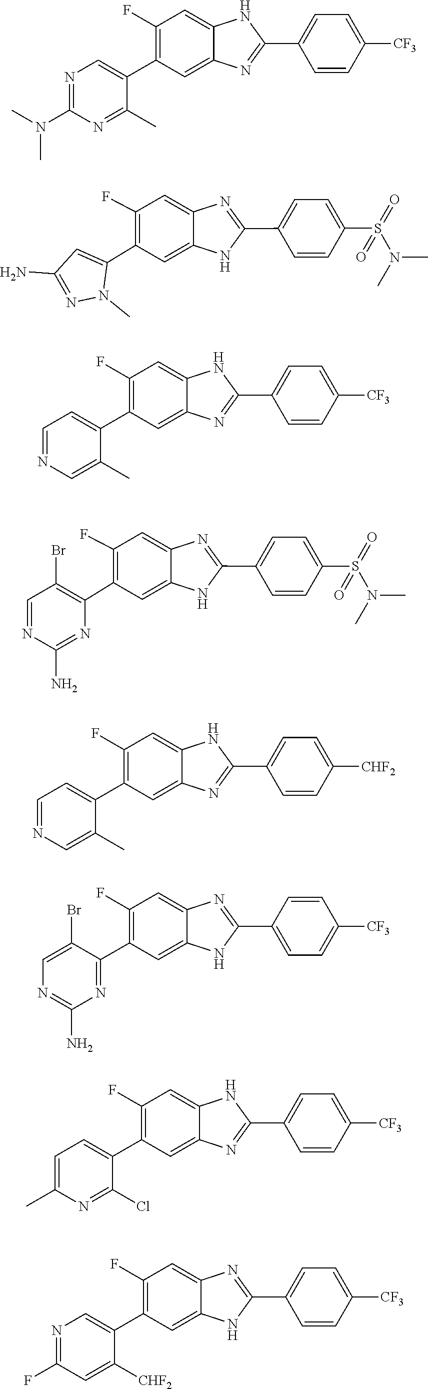

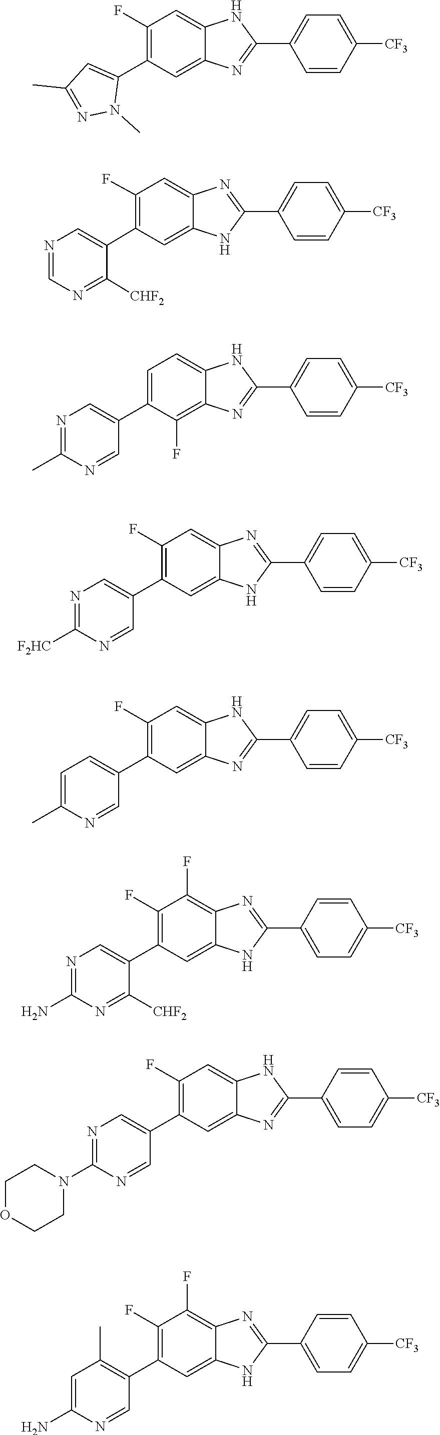

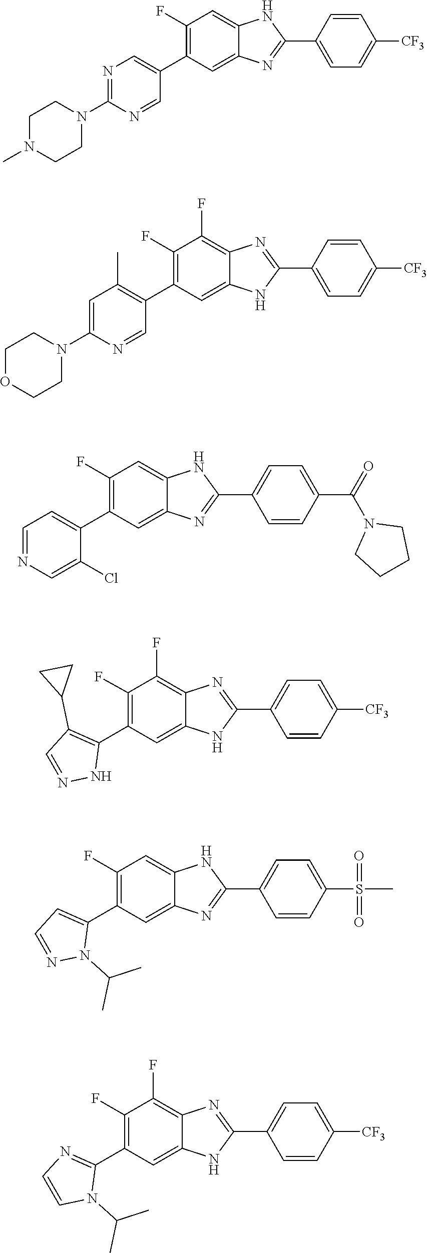 [SMILES: CN1CCN(c2ncc(-c3cc4nc(-c5ccc(C(F)(F)F)cc5)[nH]c4cc3F)cn2)CC1 ; Cc1cc(N2CCOCC2)ncc1-c1cc2[nH]c(-c3ccc(C(F)(F)F)cc3)nc2c(F)c1F ; O=C(c1ccc(-c2nc3cc(-c4ccncc4Cl)c(F)cc3[nH]2)cc1)N1CCCC1 ; Fc1c(-c2[nH]ncc2C2CC2)cc2[nH]c(-c3ccc(C(F)(F)F)cc3)nc2c1F ; CC(C)n1nccc1-c1cc2nc(-c3ccc(S(C)(=O)=O)cc3)[nH]c2cc1F ; CC(C)n1ccnc1-c1cc2[nH]c(-c3ccc(C(F)(F)F)cc3)nc2c(F)c1F]

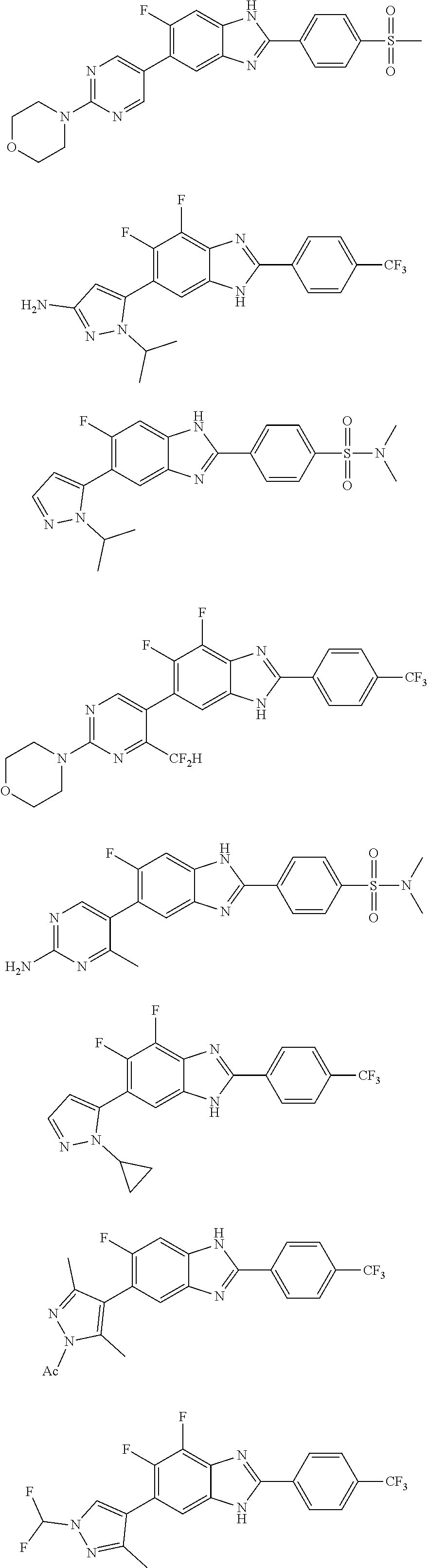

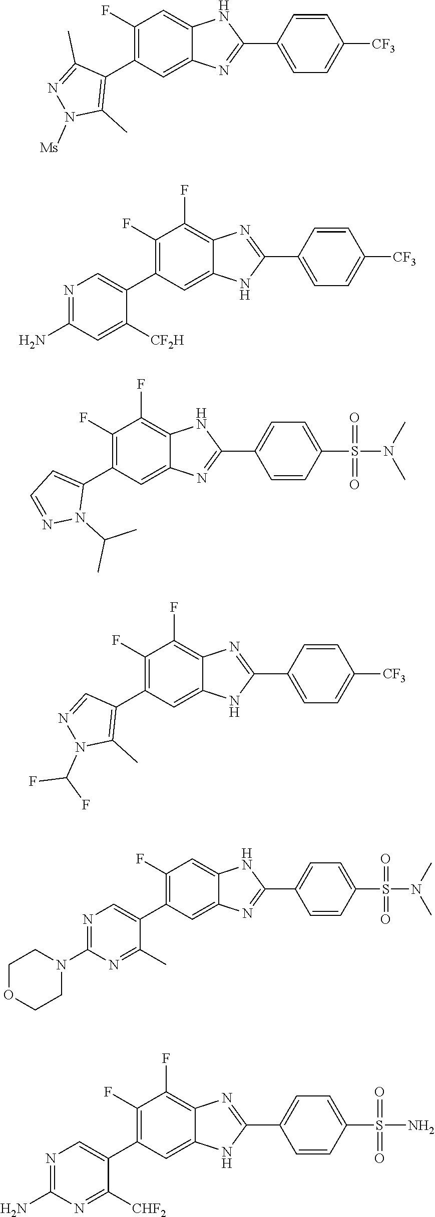

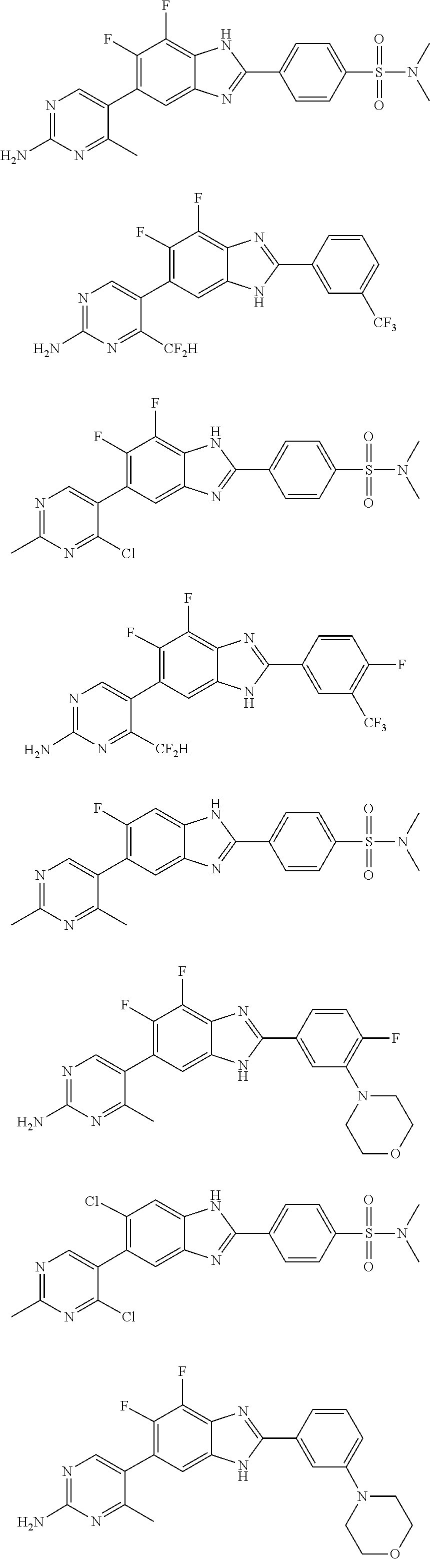

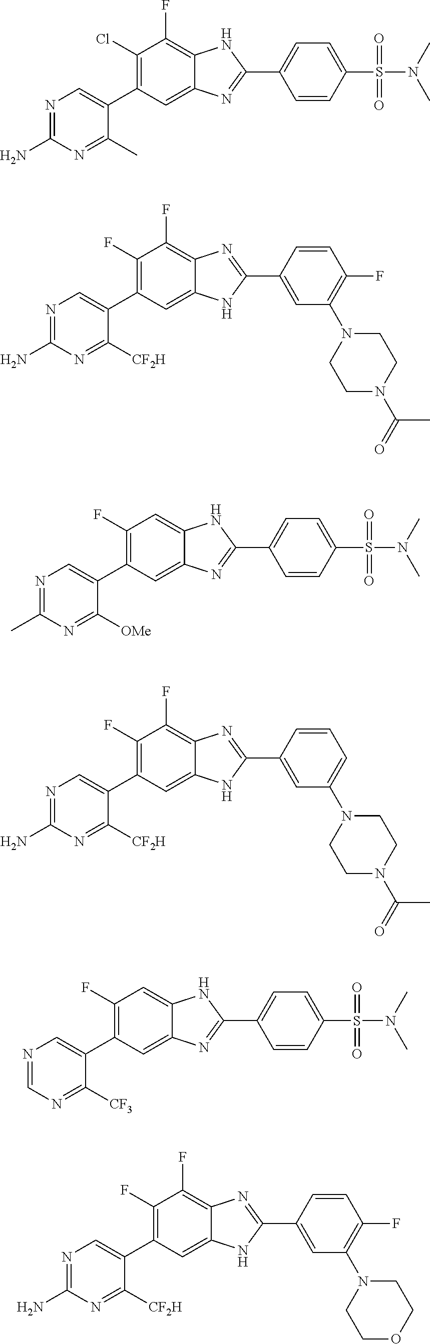

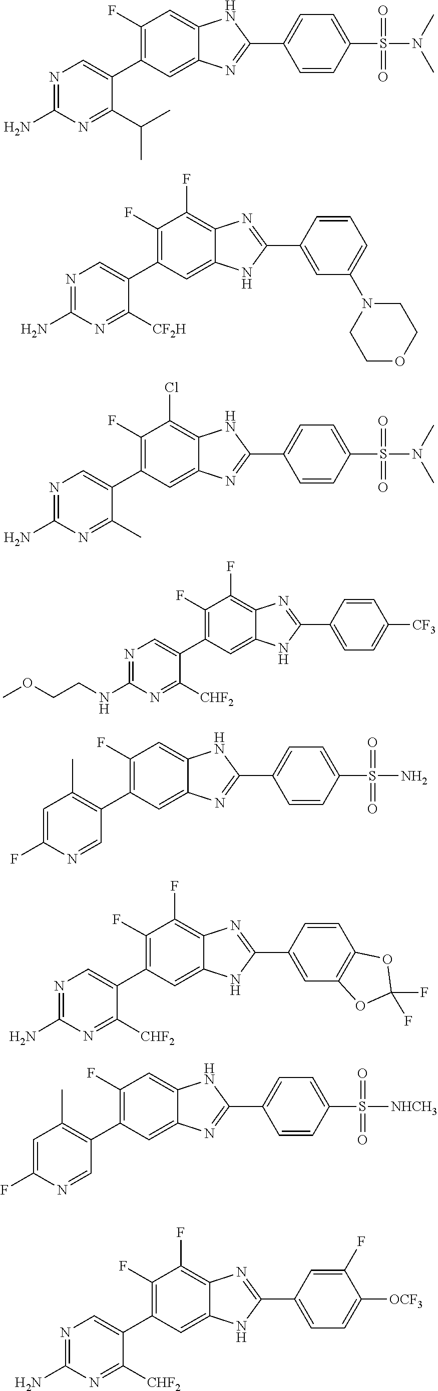

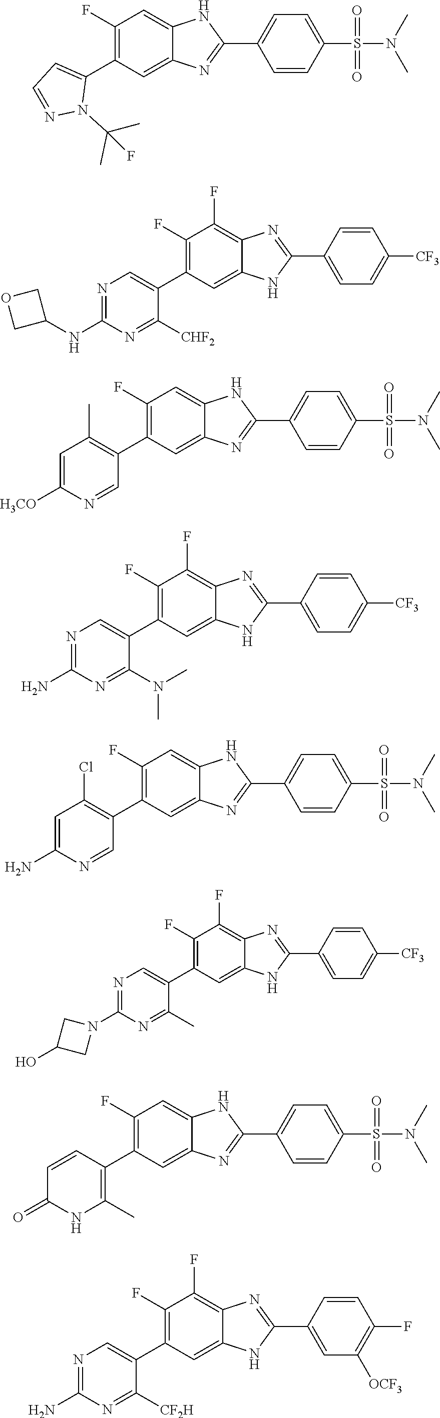

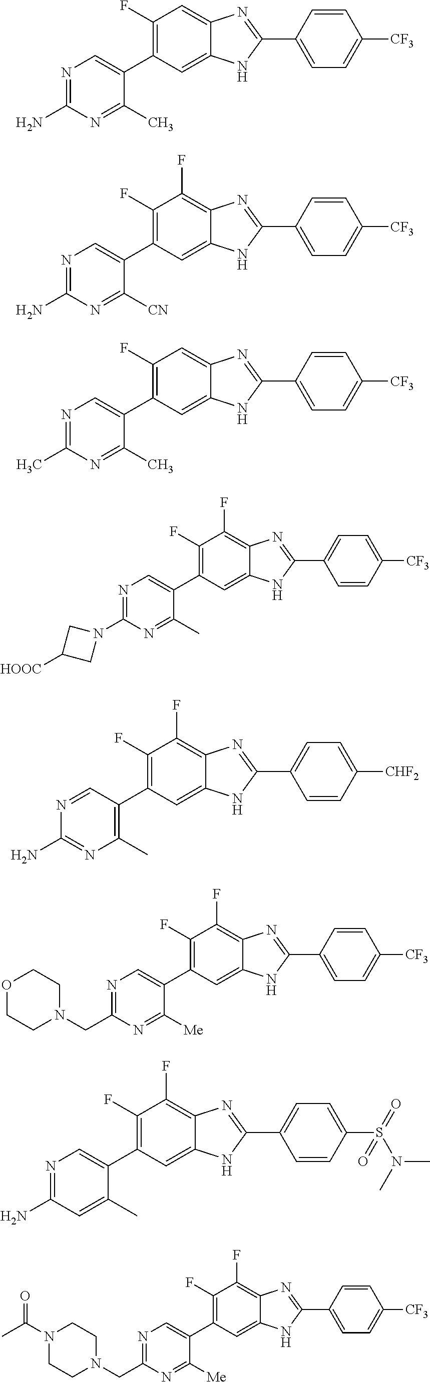

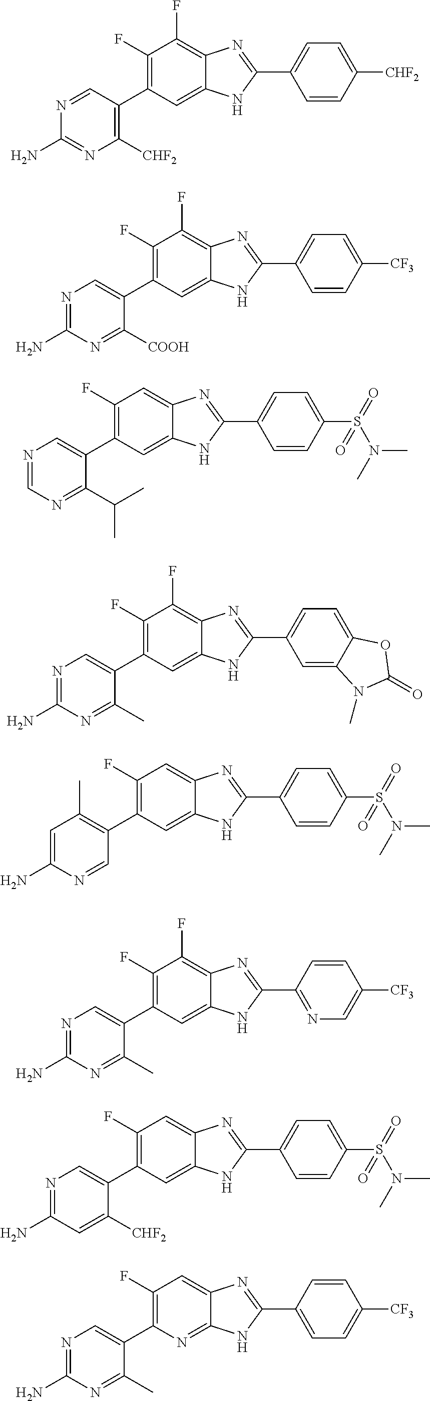

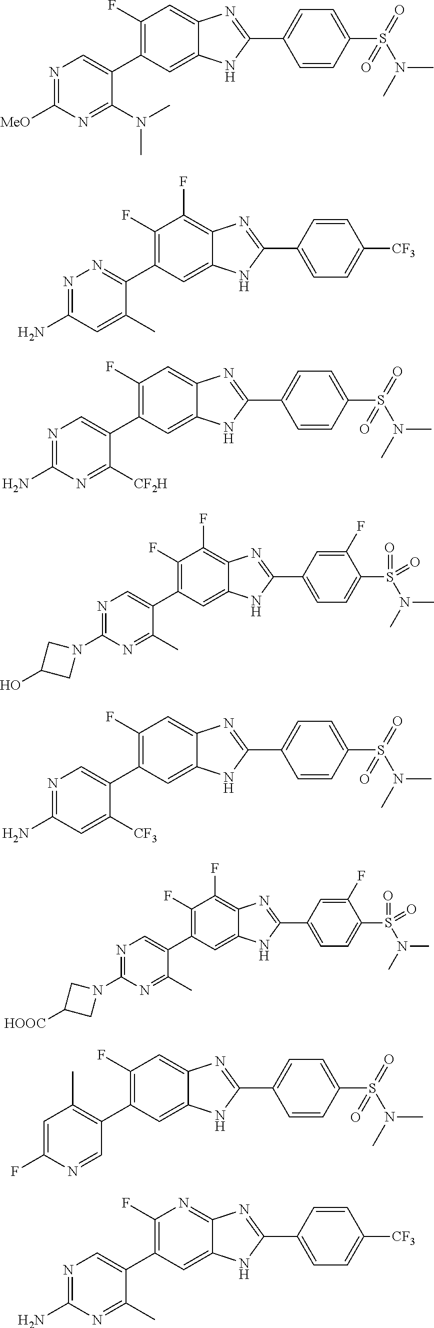

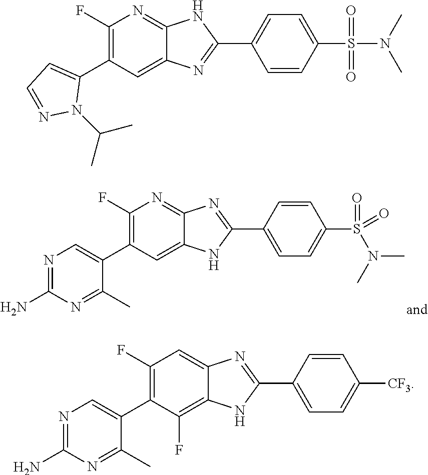

9. A method for inhibiting a soluble epoxide hydrolase comprising administering the compound according to claim 1, the stereoisomer, hydrate, solvate, pharmaceutically acceptable salt or prodrug thereof to a subject in need thereof.

10. A pharmaceutical composition comprising the compound according to claim 1, the stereoisomer, hydrate, solvate, pharmaceutically acceptable salt or prodrug thereof, and a pharmaceutically acceptable carrier, adjuvant or excipient.

11. (canceled)

12. A method for treating sEH-related or mediated disease comprising administering the compound according to claim 1, the stereoisomer, hydrate, solvate, pharmaceutically acceptable salt or prodrug thereof to a subject in need thereof.

13. The method according to claim 12, wherein the sEH-related or mediated disease is selected from: the following group: Alzheimer's disease, Parkinson's disease, Huntington's disease, dementia, cerebral ischemia, epilepsy, traumatic brain injury, stroke, Alexander disease, Alpers disease, amyotrophic lateral sclerosis, ataxia telangiectasia, Batten disease, Canavan disease, Cockayne syndrome, cortical basal degeneration, Creutzfeldt-Jakob disease, Kennedy disease, Krabbe disease, Lewy body dementia, Machado-Joseph disease, multiple sclerosis, multiple system atrophy, Pelizaeus-Merzbacher disease, Pick's disease, primary lateral sclerosis, Refsum's disease, Sandhoff disease, Schilder's disease, Tay-Sachs disease, spinocerebellar ataxia, spinal muscular atrophy, progressive supranuclear palsy, transmissible spongiform encephalopathy, tabes dorsalis, peripheral neuropathy, neuroinflammation, asthma, chronic obstructive pulmonary disease, chronic bronchitis, cystic fibrosis, atherosclerosis, restenosis after angioplasty, coronary artery disease, rheumatoid arthritis, osteoarthritis, dermatitis, eczematous dermatitis, psoriasis disease, late and chronic solid organ rejection after transplantation, systemic lupus erythematosus, dermatomyositis, polymyositis, Sjögren's syndrome, polymyalgia rheumatica, temporal arteritis, Behcet's disease, Guillain-Barré syndrome, Wegener's granulomatosis, polyarteritis nodosa, neuralgia, vasculitis, pancreatitis, ulcerative colitis, Crohn's disease, Kaposi's sarcoma, hypertension, pulmonary hypertension, adult respiratory distress syndrome, end-stage renal disease, heart failure, renal failure, liver failure, cardiac fibrosis, renal fibrosis, pulmonary fibrosis, liver fibrosis, non-alcoholic fatty liver disease, ischemic limb disease, intermittent claudication, endothelial dysfunction, male erectile dysfunction, Raynaud's disease, diabetic vascular disease, herpes zoster, gastrointestinal diseases caused by non-steroidal anti-inflammatory drugs, hand-foot syndrome caused by chemotherapy, etc.

14. A method for preparing the compound according to claim 1, wherein the method comprises the following steps:
S1: reacting a halogenated o-phenylenediamine precursor with a benzaldehyde precursor to obtain an intermediate;
S2: reacting the intermediate with a boronic acid precursor to obtain the compound;
wherein, the halogenated o-phenylenediamine precursor has a structure represented by the following general formula (1): 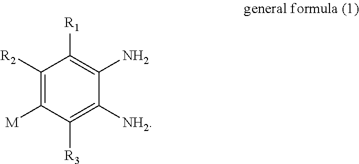
wherein, the benzaldehyde precursor has a structure represented by the following general formula (2): 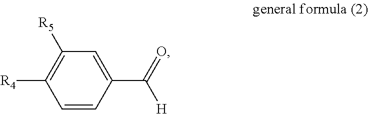
wherein, the intermediate has a structure represented by the following general formula (3): 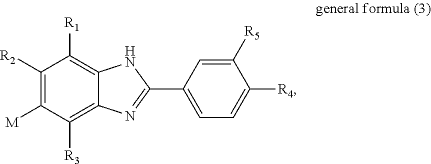
wherein, the boric acid precursor is Ar1-B(OH) 2, Ar1-BF₄K or has a structure represented by the following general formula (4): 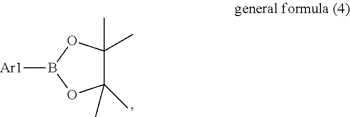
wherein R₁, R₂, R₃, R₄, R₅ and Ar1 are defined as in claim 1;
wherein, M represents a halogen.
